(19) United States
(12) Patent Application Publication
(10) Pub. No.: US 2025/0230230 A1
(43) Pub. Date: Jul. 17, 2025

(54) TREATMENT OF PERIVASCULAR FIBROSIS AND OTHER HYPERTENSIVE DISEASES AND CONDITIONS

(71) Applicant: The Brigham and Women's Hospital, Inc., Boston, MA (US)

(72) Inventor: Mark W. FEINBERG, Newton, MA (US)

(21) Appl. No.: 18/849,001

(22) PCT Filed: Mar. 23, 2023
(86) PCT No.: PCT/US2023/064866

§ 371 Date: Sep. 20, 2024

Related U.S. Application Data
(60) Provisional application No. 63/323,149, filed on Mar. 24, 2022.

(51) Int. Cl. C07K 16/24; A61K 39/00; A61K 45/06; A61P 9/00; A61P 9/12

(52) U.S. Cl. CPC C07K 16/244; A61K 45/06; A61P 9/00; A61P 9/12; A61K 2039/505

(57) ABSTRACT

The disclosure features methods and compositions for the treatment of perivascular fibrosis and other hypertensive diseases and conditions, cardiovascular diseases, and chronic kidney disease.

### CROSS-REFERENCE TO RELATED APPLICATIONS

This application claims priority to U.S. Provisional Application No. 63/323,149, filed Mar. 24, 2022, which is incorporated herein by reference in its entirety.

### SEQUENCE LISTING

The instant application contains a Sequence Listing which has been submitted electronically in XML format and is hereby incorporated by reference in its entirety. Said XML copy, created on Mar. 21, 2023, is named 51665-002WO2_Sequence_Listing_3_21_23 and is 63,417 bytes in size.

### FIELD OF THE INVENTION

The present invention is related to reducing fibrosis (e.g., perivascular fibrosis) and treatment of hypertensive diseases and conditions, cardiovascular diseases, and chronic kidney disease.

### BACKGROUND OF THE INVENTION

Perivascular fibrosis is a hallmark of advanced vascular disease states often associated with elevated blood pressure (BP), vascular stiffness, adverse vascular remodeling, and end-organ dysfunction such as in the heart and kidney. Continuous excessive stress and inflammation during hypertension increase the amount of perivascular connective tissue and collagen deposition that is characteristic of pathological perivascular fibrosis. Indeed, hypertension remains a serious public health problem that contributes to considerable global cardiovascular disease and premature death worldwide by increasing the risk of ischemic heart disease, stroke, and chronic kidney disease. Early management and therapeutic intervention may greatly reduce the occurrence or postpone the development of chronic fibrotic diseases, especially hypertensive cardiovascular, kidney, and aortic disease. However, therapeutic targets that specifically control perivascular fibrosis are not well defined.

### SUMMARY OF THE INVENTION

The methods and compositions disclosed herein address an unmet need in the art.

Other features and advantages of the invention will be apparent from the following Detailed Description and the Claims.

In a first aspect, the invention features a method for treating a hypertensive disease or condition, a cardiovascular disease, or chronic kidney disease in a subject in need thereof, the method including administering an effective amount of an anti-interleukin-9 (IL-9) antibody, or an antigen-binding fragment thereof, to the subject, thereby treating the hypertensive disease or condition, the cardiovascular disease, or the chronic kidney disease.

In another aspect, the invention features an anti-IL-9 antibody, or an antigen-binding fragment thereof, for use in treating a hypertensive disease or condition, a cardiovascular disease, or chronic kidney disease in a subject in need thereof, and/or reducing fibrosis (e.g., perivascular fibrosis) in a subject in need thereof.

In another aspect, the invention features a method for reducing fibrosis in a subject in need thereof, the method including administering an effective amount of an anti-IL-9 antibody, or an antigen-binding fragment thereof, to the subject, thereby reducing fibrosis in the subject. In some embodiments, the fibrosis includes perivascular fibrosis. In some embodiments, the perivascular fibrosis occurs in the subject's aorta, heart, and/or kidney. In some embodiments, the subject has a hypertensive disease or condition, a cardiovascular disease, or a chronic kidney disease.

In some embodiments of any of the foregoing aspects, the hypertensive disease or condition is a heart disease or a kidney disease. In some embodiments, the hypertensive disease or condition includes hypertension; hypertensive heart disease; heart failure with preserved ejection fraction; coronary heart disease; hypertensive-associated end organ damage; or any combination thereof; the cardiovascular disease includes coronary artery disease, atherosclerosis, myocardial infarction, heart failure, atrial fibrillation, cerebrovascular disease, stroke, peripheral artery disease, aortic aneurysm, retinopathy, or any combination thereof; or the chronic kidney disease includes end stage renal disease (ESRD). In some embodiments, the hypertension includes isolated systolic, malignant, or resistant hypertension.

In some embodiments of any one of the foregoing aspects, the method results in: a decreased expression level of Alox15 and/or Haptoglobin (Hp) in a sample obtained from the subject relative to a reference expression level of Alox15 and/or Hp; and/or a decreased expression level of one or more fibrotic genes in a sample obtained from the subject relative to a reference expression level of the one or more fibrotic genes. In some embodiments, the one or more fibrotic genes includes Alox15, Col8a1, Mmp2, Fmod, and/or Angptl1.

In some embodiments of any one of the foregoing aspects, the method results in an improvement in heart function, kidney function, or vascular remodeling compared to a subject who has not been treated with the anti-IL-9 antibody or the antigen-binding fragment thereof.

In some embodiments of any one of the foregoing aspects, the method results in: a decreased fibroblast intracellular calcium mobilization; a decreased fibroblast activation or differentiation; a reduced production of one or more extracellular matrix (ECM) components; an improved left ventricular global longitudinal strain (LV GLS); a decreased pulse wave velocity (PWV);
an increased circumferential (Circ) strain; a decreased ratio of albumin to creatinine; a decreased kidney injury molecule-1 (KIM-1) expression level; a decreased calcium deposition in the perivascular adventitia; or any combination of the above improvements, compared to a subject who has not been treated with the anti-IL-9 antibody or the antigen-binding fragment thereof. In some embodiments, the one or more ECM components includes collagen.

In some embodiments of any one of the foregoing aspects, the anti-IL-9 antibody is an anti-human IL-9 antibody. In some embodiments, the anti-IL-9 antibody includes enokizumab.

In some embodiments of any one of the foregoing aspects, the anti-IL-9 antibody, or the antigen-binding fragment thereof, is administered to the subject as a monotherapy.

In some embodiments of any one of the foregoing aspects, the anti-IL-9 antibody, or the antigen-binding fragment thereof, is administered to the subject in combination with one or more additional therapeutic agents. In some embodiments, the one or more additional therapeutic agents include an antihypertensive agent, an anti-arrhythmic agent, an anticoagulant agent, an anti-platelet agent, a cholesterol-lowering agent, digoxin, a nitrate, or any combination thereof. In some embodiments, the anti-hypertensive agent includes an angiotensin II receptor antagonist, an angiotensin-converting enzyme (ACE) inhibitor, a diuretic, a calcium channel antagonist, an adrenergic receptor antagonist, a vasodilator, a renin inhibitor, an aldosterone receptor antagonist, an alpha-2 adrenergic receptor agonist, an endothelin receptor blocker, or any combination thereof.

In some embodiments of any one of the foregoing aspects, the subject is a human.

In another aspect, the invention features a kit including an anti-IL-9 antibody, or an antigen-binding fragment thereof, and a package insert including instructions to administer the anti-IL-9 antibody, or the antigen-binding fragment thereof, to a subject to treat a hypertensive disease or condition, a cardiovascular disease, or chronic kidney disease in a subject in need thereof, and/or reduce fibrosis (e.g., perivascular fibrosis) in a subject in need thereof.

In another aspect, the invention features a method for treating a hypertensive disease or condition, a cardiovascular disease, or chronic kidney disease in a subject in need thereof, the method including administering an effective amount of a Kruppel-like factor 10 (KLF10) agonist to the subject, thereby treating the hypertensive disease or condition, the cardiovascular disease, or the chronic kidney disease.

In another aspect, the invention features a KLF10 agonist for use in treating a hypertensive disease or condition, a cardiovascular disease, or chronic kidney disease in a subject in need thereof, and/or reducing fibrosis (e.g., perivascular fibrosis) in a subject in need thereof.

In another aspect, the invention features a method for reducing fibrosis in a subject in need thereof, the method including administering an effective amount of a KLF10 agonist to the subject, thereby reducing fibrosis in the subject. In some embodiments, the fibrosis includes perivascular fibrosis.

In some embodiments, the perivascular fibrosis occurs in the subject's aorta, heart, and/or kidney. In some embodiments, the subject has a hypertensive disease or condition, a cardiovascular disease, or a chronic kidney disease.

In some embodiments of any one of the foregoing aspects, the hypertensive disease or condition is a heart disease or a kidney disease. In some embodiments, the hypertensive disease or condition includes hypertension; hypertensive heart disease; heart failure with preserved ejection fraction; coronary heart disease; hypertensive-associated end organ damage; or any combination thereof; the cardiovascular disease includes coronary artery disease, atherosclerosis, myocardial infarction, heart failure, atrial fibrillation, cerebrovascular disease, stroke, peripheral artery disease, aortic aneurysm, retinopathy, or any combination thereof; or the chronic kidney disease includes ESRD. In some embodiments, the hypertension includes isolated systolic, malignant, or resistant hypertension.

In some embodiments of any one of the foregoing aspects, the KLF10 agonist includes a small molecule agonist, recombinant KLF10, or a viral vector (e.g., adeno-associated viral vector) including a nucleic acid encoding KLF10. In some embodiments of any one of the foregoing aspects, the KLF10 agonist is administered to the subject as a monotherapy. In some embodiments of any one of the foregoing aspects, the KLF10 agonist is administered to the subject in combination with one or more additional therapeutic agents. In some embodiments, the one or more additional therapeutic agents include an antihypertensive agent, an anti-arrhythmic agent, an anticoagulant agent, an anti-platelet agent, a cholesterol-lowering agent, digoxin, a nitrate, or any combination thereof. In some embodiments, the anti-hypertensive agent includes an angiotensin II receptor antagonist, an ACE inhibitor, a diuretic, a calcium channel antagonist, an adrenergic receptor antagonist, a vasodilator, a renin inhibitor, an aldosterone receptor antagonist, an alpha-2 adrenergic receptor agonist, an endothelin receptor blocker, or any combination thereof. In some embodiments of any one of the foregoing aspects, the subject is a human.

In another aspect, the invention features a kit including a KLF10 agonist and a package insert including instructions to administer the KLF10 agonist to a subject to treat a hypertensive disease or condition, a cardiovascular disease, or chronic kidney disease in a subject in need thereof, and/or reduce fibrosis (e.g., perivascular fibrosis) in a subject in need thereof.

### BRIEF DESCRIPTION OF THE DRAWINGS

FIG. 1A-FIG. 1G show that KLF10 expression is increased in T cells after Ang II treatment. A, Volcano plot highlighting regulated transcription factors in peripheral blood mononuclear cells (PBMCs) comparing hypertensive patients with normal left ventricular (LV) size and healthy controls (left), and hypertensive patients with LV remodeling and healthy controls (right). B, Venn diagram indicating the number of transcription factors regulated from two comparisons (left), and the normalized reads of KLF10 in PBMCs from the healthy control group (n=8), hypertensive patients with or without LV remodeling (n=14 in each group). C, Normalized reads of KLF10 in PBMCs from controlled (n=11) and uncontrolled hypertensives (n=9). D, Volcano plot highlighting regulated transcription factors in splenic CD3+ T cells (left), and the normalized reads of KI/10 in splenic CD3+ T cells in sham and Ang II-treated mice. E, Schematic diagram of CD3−, CD3+ and CD4+ cell isolation from PBS or Ang II treated C57BL/6 mice, and the mRNA expression of KI/10 in different cells (n=5). F, Representative immunofluorescence staining, and the number of CD4+KLF10+ T cells in the adventitial regions in PBS or Ang II treated groups (n=5, scale bars=50 μm). G, the expression of Kif10 in Ang II treated CD4+ T cells. P values correspond to one-way ANOVA with Tukey's multiple comparisons tests (B, G), or unpaired two-tailed t tests (C, D) for normal distributed data. E and F, P values correspond to unpaired two-tailed Mann-Whitney U-tests. LV indicates left ventricle; Con, control; Nor, normal LV size; Re, remodeling; WT, wild type; Ang II, Angiotensin II.

FIG. 2A-FIG. 2K show that KLF10 deficiency in CD4+ T cells impairs the function of hypertension-related organs and triggers perivascular fibrosis independent of blood pressure. A, Schematic diagram of experimental set-up of mice groups treated with PBS or Ang II infusion. B, Aortic blood pressure in Cre and TKO mice with PBS (n=4) or Ang II (n=6) treatment for 28 days or 42 days. C, Global longitudinal strain (GLS) in Cre and TKO mice before and after Ang II treatment (n=6 in Cre mice, n=7 in TKO mice, and n=10 in Ang II groups). D-E, Representative ultrasound imaging of the suprarenal abdominal aorta, and measurements of pulse wave velocity (PWV, D) and circumferential strain in Cre (n=8) and TKO mice (n=10) after 28 days of Ang II treatment. F-G, The ratio of albumin and creatinine (F), and the level of kidney injury molecule (KIM)-1 (G) in urine from Cre (n=7) and TKO mice (n=6) after Ang II treatment. H-I, Representative images of Masson trichrome staining, and quantification of perivascular fibrosis in the heart (H, scale bars=100 μm) and kidney (1, scale bars=200 μm) (n=10). J-K, Representative images of Masson trichrome staining, and Sirius red staining and quantification of perivascular fibrosis and adventitial collagen in the aorta (n=5, scale bars=200 μm). B-I, P values correspond to unpaired two-tailed t tests for normal distributed data. J and K, P values correspond to unpaired two-tailed Mann-Whitney U-tests. Ang II indicates angiotensin II; Echo, echocardiogram; AOP, aortic pressure; ns, not significant; GLS, global longitudinal strain; PWV, pulse wave velocity; Circ Strain, circumferential strain; KIM-1, kidney injury molecule-1.

FIG. 3A-FIG. 3O show that KLF10 deficient CD4+ T cells release IL-9 that mediates perivascular fibrosis. A, IL-9 levels were measured in plasma in PBS or Ang II treated Cre and TKO mice (n=4 in PBS groups, and n=8 in Ang II-treated groups). B, mRNA expression level of 119 in the PBMCs, heart, kidney, and aorta in Ang II-treated Cre and TKO mice (n=6 in aortic groups, and n=8 in others). C-D, Representative flow cytometry plots and the percentage of CD4+IL9+ T cells gated in CD3+ T cells in the aorta (C, n=5) and spleen (D, n=5). E, Representative immunofluorescence staining, and the number of CD4+IL9+ T cells in the aorta in Ang II treated Cre and TKO mice (n=5, scale bars=50 μm). F-G, Schematic diagram of the experimental setup for CD4+ T cell isolation from Ang II-treated Cre or TKO mice (F), and quantification of 119 mRNA in isolated CD4+ T cells, and IL-9 protein released into the supernatants of CD4+ T cells (G, n=6). H-I, Schematic diagram of the experimental setup of CD4+ T cell isolation from Cre control mice for in vitro treatment (H), quantification of 119 mRNA in the treated CD4+ T cells, and IL-9 protein released into the supernatants of treated CD4+ T cells (1). J, Schematic diagram of the experimental setup for recombinant mIL-9 treatment in Cre mice. K, GLS in mIL-9 or PBS treated Cre mice after Ang II infusion (n=6). L, the value of PWV and circumferential strain in mIL-9 or PBS treated Cre mice after 28 days Ang II treatment (n=6). M, the ratio of albumin and creatinine, and the level of KIM-1 in urine in mIL-9 or IgG treated Cre mice after 28 days Ang II treatment (n=6). N-O, Representative images of Masson trichrome staining and Sirius red staining, and the area of perivascular fibrosis and adventitial collagen in the aorta (n=6, scale bars=200 μm). P values correspond to two-way ANOVA with Tukey's multiple comparisons tests (A, and 1), or unpaired two-tailed t tests (B, G, and K-O) for normal distributed data. C-E, P values correspond to unpaired two-tailed Mann-Whitney U-tests. PBMC indicates peripheral blood mononuclear cells; Ang II, angiotensin II; GLS, global longitudinal strain; PWV, pulse wave velocity; Circ Strain, circumferential strain; KIM-1, kidney injury molecule-1.

FIG. 4A-FIG. 4H show that KLF10 binds to the IL-9 promoter and interacts with HDAC to inhibit IL-9 activation. A, Putative KLF10 transcription factor-binding sites in the mouse IL-9 promoter region. The putative binding sequences are highlighted. The transcription initiation site is defined as +1. B, Primary CD4+ T cells were isolated from Cre control mice and subjected to the chromatin immunoprecipitation (ChIP) assay with antibodies against IgG or KLF10. The immunoprecipitated DNA was subjected to semiquantitative PCR and q-PCR. C, The protein level of KLF10 after transfection. D-E, HEK293T cells were transfected with a full-length mouse IL-9 promoter luciferase reporter or 5′ truncations of the IL-9 promoter, together with expression plasmids encoding full-length mouse KLF10 or empty vector (mock control). F, Primary CD4+ T cells were treated with Ang II for 12 h, then cells were harvested, lysed, and subjected to immunoprecipitation by the indicated antibodies. Immunoprecipitation (IP) were subjected to Western blotting with the indicated antibodies, and the quantification of IP. G-H, HEK293T were co-transfected with mouse KLF10 expression vector and the indicated mouse IL-9 promoter luciferase reporters (WT, Mut1, Mut2, or Mut3), and siRNA (non-specific or HDAC1), and the relative luminescence units (RLU) after transfection. B, and F, P values correspond to unpaired two-tailed Mann-Whitney U-tests. For normal distributed data, P values correspond to unpaired two-tailed t tests (D, and G), or two-way ANOVA with Tukey's multiple comparisons tests (E, and H). Ctrl indicates control; Ang II, angiotensin II; Luc, luciferase reporters; RLU, relative luminescence units.

FIG. 5A-FIG. 5G show transcriptomic changes involved in Ang II-induced perivascular fibrosis. A, GOChord plot showing the significantly regulated genes (log 2 fold change >1.5; FDR <0.05) involved in the top 7 enriched pathways in non-stripped aorta. B, Differentially expressed genes in stripped and non-stripped aortas in Ang II treated Cre and TKO mice (FDR, <0.05). C, IPA Canonical pathway analysis after overlapping differentially expressed genes in stripped and non-stripped aortas. D, IPA Canonical pathway analysis by using unique differentially expressed genes in non-stripped aorta. E, Differentially expressed calcium pathway related genes. F, Representative images of Von Kossa staining, and the area of calcium deposition in the aorta (n=5, scale bars=100 μm). G, Real-time changes in intracellular calcium flux following IL-9, Ang II or Ang II and IL-9 treatment (Scale bars=50 μm). F, P values correspond to unpaired two-tailed Mann-Whitney U-tests. Ang II indicates angiotensin II, Cont, Control.

FIG. 6A-FIG. 6G show that TKO fibroblasts display an activation signature and IL-9 and Ang II treatment recapitulate the phenotype in control fibroblasts. A, Schematic diagram of fibroblast isolation from Ang II-treated Cre or TKO mice. B, Representative immunofluorescence images, and the mean fluorescence intensity of Col1a1 and α-SMA in the isolated fibroblasts from Ang II-treated Cre or TKO mice (n=6, scale bars=50 μm). C, Heatmap of dysregulated genes related to myofibroblast markers, fibroblast activation signature, and calcium signaling in isolated fibroblasts. D, Schematic diagram of primary aortic fibroblast isolation from C57BL/6 mice for further in vitro experiment. E, Gene expression of fibrotic markers in primary aortic fibroblasts grown in supernatants from KO or Cre CD4+ T cells treated with Ang II in the presence of IgG or anti-IL-9 monoclonal antibodies (mAbs). F, Gene expression of fibrotic markers in primary aortic fibroblasts after treatment with IL-9, Ang II, or Ang II and IL-9 treatment. G, Representative immunofluorescence images, and the mean fluorescence intensity of Col1a1 and α-SMA in primary aortic fibroblasts after IL-9, Ang II, or Ang II and IL-9 treatment (n=6, scale bars=50 μm). P values correspond to an unpaired two-tailed t test (B), one-way ANOVA with Tukey's multiple comparisons tests (G) for normal distributed data. Ang II indicates angiotensin II.

FIG. 7A-FIG. 7M show that single-cell RNA sequencing revealed fibroblast heterogeneity and activation signatures induced in TKO aortas. A, Schematic diagram of aortic cells isolated from Ang II-treated Cre or TKO mice for single-cell RNA sequencing. B, Uniform Manifold Approximation and Projection (UMAP) of different aortic cell types. C, the percentage of different aortic cell types. D, IPA pathway analysis using total differentially expressed genes in fibroblasts. E, UMAP of 9 main fibroblast cell clusters. F, the number of different fibroblast clusters in Ang II treated Cre and TKO mice. G, dot plot of fibroblast activation signature-related genes. H, the UMAP of fibrosis genes by using add module score analysis. I, Velocity vector field displayed over the FBS UMAP. J, K-means cluster analysis for each fibroblast subclusters. K, Pathway analysis using the specific genes enriched in FBS_8. L, Representative immunofluorescence images of PDGFRa and Col8a1 in aorta (n=5, scale bars=50 μm). M, Overlapping upregulated genes from the indicated single-cell RNA seq dataset and isolated fibroblast bulk-RNA seq datasets (top); the overlapping increased genes from single-cell RNA seq (bottom). L, P values correspond to an unpaired two-tailed Mann-Whitney U-test. DC indicates dendritic cells; FBS, fibroblasts; EC, endothelial cells; RBC, red blood cells; VSMC, vascular smooth muscle cells; Ang II, angiotensin II.

FIG. 8A-FIG. 8K show that neutralization of endogenous IL-9 reversed the Ang II-induced perivascular fibrosis and ameliorated injury of hypertension-related organs. A, Schematic diagram of the experimental setup for treatment with anti-IL-9 antibodies (mAb) in PBS or Ang II treated-TKO mice. B-C, Quantification of GLS (B), PWV and Circ Strain (C) in anti-IL-9 antibodies (mAb) or IgG treated TKO mice after 28 days PBS (n=4) or Ang II (n=5) infusion. D, The ratio of albumin and creatinine, and the level of KIM-1 in urine (n=4 in PBS groups, and n=5 in Ang II groups). E-F, Representative images of Masson trichrome staining and Sirius red staining, and quantification of perivascular fibrosis and adventitial collagen in the aorta (n=4 in PBS groups, n=6 in Ang II groups, scale bars=200 μm). G, Venn diagram of overlapping dysregulated genes from the upregulated genes from the isolated fibroblast bulk-RNA seq dataset and the downregulated genes in the anti-IL-9 mAb or IgG treated TKO mice bulk-RNA seq datasets (top), and the heatmap of downregulated genes in IL-9 mAb and IgG treated TKO mice non-stripped aortas after overlapping (bottom). H, Venn diagram of overlapping dysregulated genes from upregulated genes in the single cell-RNA seq dataset and the downregulated genes in the anti-IL-9 mAb or IgG treated TKO mice bulk-RNA seq datasets (top), the increased overlapping genes from single-cell seq (bottom), and the heatmap of downregulated overlapping genes in anti-IL-9 mAb and IgG treated of non-stripped aortas from TKO mice (right). I, quantification of PWV and Circ Strain in anti-IL-9 mAb (n=4) or IgG (n=5) treated WT mice after 28 days of Ang II treatment. J-K, Representative images of Masson trichrome staining and Sirius red staining, and quantification of perivascular fibrosis and adventitial collagen in the aorta in anti-IL-9 mAb (n=10) or IgG (n=6) treated WT mice after 28 days of Ang II treatment (scale bars=200 μm)., P values correspond to two-way ANOVA with Tukey's multiple comparisons tests (B-F), or unpaired two-tailed t tests (I-K) for normal distributed data. Ang II indicates angiotensin II; PWV, pulse wave velocity; Circ Strain, circumferential strain; KIM-1, kidney injury molecule-1.

FIG. 9A-FIG. 9J show assessment of end-organ injury and perivascular fibrosis in Cre and TKO mice in the absence of Ang II. A-B, quantification of pulse wave velocity (PWV, A) and circumferential (circ) strain (B) in Cre and TKO mice prior to Ang II treatment (n=6). C-D, Relaxation or contraction in response to increasing doses of sodium nitroprusside (SNP) or phenylephrine (PE) in mesenteric vessels (n=6 in each group). E-F, The ratio of albumin and creatinine (E), and the expression of kidney injury molecule (KIM)-1 (F) in urine from Cre and TKO mice (n=6) prior to Ang II treatment. G, representative images of Masson trichrome staining (MTS), and quantification of total fibrosis in the heart (n=10, scale bars=500 μm). H, representative images of H&E and wheat germ agglutinin (WGA) staining of heart sections, and quantification of myocyte cross-sectional area (n=5, scale bars=100 μm). I, Representative images of MTS, and quantification of fibrosis in the aorta before Ang II treatment in male mice (n=8, scale bars=200 μm). J, Representative images of MTS, and quantification of fibrosis in the aorta after Ang II treatment for 28 days in female mice (n=5, scale bars=200 μm). P values correspond to unpaired two-tailed t tests (A, B, E, F, G, and 1), or two-way ANOVA with Tukey's multiple comparisons test (C and D) for normal distributed data. H, and J, P values correspond to unpaired two-tailed Mann-Whitney U-tests. PWV indicates pulse wave velocity; SNP, sodium nitroprusside; PE, phenylephrine; KIM-1, kidney injury molecule 1.

FIG. 10A-FIG. 10N show cytokine profiling from Cre and TKO mice. A-B, gene expression of angiotensin II receptors in CD4+ T cells and fibroblasts (n=5 in each group). C, Circular heatmap of cytokine profiling comparing plasma from Ang II-treated male Cre and TKO mice. D, quantification of IL-9 protein level in plasma in Ang II-treated female mice (n=5 in each group). E-I, Flow cytometric analyses of aortic cells from Ang II-treated Cre and TKO mice (E), and the percentages of different cell subgroups in the aorta (F, G) and spleen (H, 1) after Ang II treatment (n=5 in each group). J, the evaluation of IL-9 antibody specificity in non-stripped aorta showing by positive control, IgG Isotype control and secondary antibody only control (scale bar=50 μm). K-L, quantification of IL-13, IL-5, and IL-4 protein levels in plasma of Ang II-treated TKO or Cre male (n=8 in each group) and female (n=5 in each group) mice. M, Circular heatmap of cytokine profiling comparing the supernatants from isolated primary CD4+ T cells from Ang II-treated Cre and TKO mice. N, quantification of IL-5 and IL-4 protein levels in the supernatants from isolated primary CD4+ T cells from Ang II-treated Cre and TKO mice (n=5 in each group). K, P values correspond to unpaired two-tailed t tests for normal distributed data. A, B, D, F, G-I, K, N, P values correspond to unpaired two-tailed Mann-Whitney U-tests.

FIG. 11A-FIG. 11F show transcriptomic changes in stripped aortas of Ang II treated Cre and TKO mice. A-C, PCA plot of gene expression (A), heatmap and hierarchical clustering (B), and volcano plot of differentially expressed genes (log 2 fold change, >1.5 and FDR, <0.05) (C) in stripped aortas from Ang II-treated Cre and TKO mice. D, the heatmap of top 25 dysregulated genes in stripped aortas from Ang II-treated Cre and TKO mice. E, Chord plot highlighting top dysregulated genes contributing to dysregulated signaling pathways. F, IPA canonical pathway analysis in stripped aortas from Ang II-treated Cre and TKO mice.

FIG. 12A-FIG. 12F show transcriptomic changes in non-stripped aortas of Ang II treated Cre and TKO mice. A-C, PCA plot of gene expression (A), heatmap and hierarchical clustering (B), and volcano plot of differentially expressed genes (log 2 fold change, >1.5 and FDR, <0.05) (C) in non-stripped aortas from Ang II-treated Cre and TKO mice. D, heatmap of top 25 dysregulated genes in non-stripped aortas from Ang II-treated Cre and TKO mice. E, IPA canonical pathway analysis in non-stripped aortas from Ang II-treated Cre and TKO mice. F, Circular heatmap of dysregulated genes from non-stripped aortas of Ang II-treated Cre and TKO mice revealed enrichment for the calcium pathway.

FIG. 13A-FIG. 13B show a comparison of gene signatures from stripped and non-stripped aortas from Ang II treated Cre and TKO mice. A, Overlapping dysregulated genes from stripped and non-stripped aortic datasets after Ang II treatment of Cre and TKO mice (top), and the heatmap of top 25 dysregulated overlapping genes (bottom). B, unique genes in non-stripped aortas after Ang II treatment of Cre and TKO mice (top), and heatmap of the top 25 dysregulated genes (bottom).

FIG. 14A-FIG. 14F show isolated fibroblasts for RNA-seq and treatment in vitro. A, the evaluation of Col1 antibody specificity in isolated aortic fibroblasts showing by positive control, IgG Isotype control and secondary antibody only control (scale bar=50 μm). B-D, Primary aortic fibroblasts were isolated from non-stripped aortas of Ang II-treated Cre and TKO mice. Analyses are shown for PCA plot of gene expression (B), heatmap and hierarchical clustering (C), and volcano plot of differentially expressed genes (log 2 fold change, >1.5 and FDR, <0.05) (D). E, Normalized reads of different gene transcripts in isolated CD45-CD90.2+ cells. F, expression of 119r in isolated CD4+ T cells (left); normalized reads of 119r in the fibroblast RNA-seq dataset (FIGS. 14B-14D), in the stripped or non-stripped aorta RNA-seq datasets (FIGS. 11 and 12), and in the isolated T cell RNA-seq dataset (GSE143809) from Ang II treated C57BL/6 mice. F, P values correspond to unpaired two-tailed Mann-Whitney U-tests.

FIG. 15A-FIG. 15B show expression of TGF-β in the plasma and CD4+ T cell supernatants. A, Expression level of TGF-β1-3 in the plasma from Cre and TKO mice after Ang II or PBS treatment. B, the expression level of TGF-β1-3 in WT or KO CD4+ T cell supernatants. A-B, P values correspond to unpaired Mann-Whitney test (A) with Bonferroni-Dunn's multiple comparisons tests (B).

FIG. 16A-FIG. 16C show quality control metrics and resolutions from single cell RNA sequencing (scRNA-seq). A, Cell numbers of each sample after filtering in single-cell RNA sequencing. B, Elbow plot visualization of the standard deviation of each principal component. C, Heatmaps showing the top genes driving the first 9 principal components.

FIG. 17A-FIG. 17E show identification of cell clusters present in the mouse aortas of Ang II treated Cre and TKO mice by single cell RNA sequencing (scRNA-seq). A, heatmap of enriched genes for each sub-cluster of aortic cells. B, UMAP for each sample after renaming clusters with the indicated cell types. C-D, dot plot (C) and representative UMAP (D) of specific gene markers in different aortic cell populations of Cre and TKO Ang II-treated mice. E, Number of indicated cell types in Ang II-treated TKO aortas compared to Cre controls.

FIG. 18A-FIG. 18D show identification of fibroblast cell clusters present in the mouse aortas of Ang II treated Cre and TKO mice by single cell RNA sequencing (scRNA-seq). A, Violin plot of Pdgfra expression in different aortic cell populations showing enrichment in fibroblasts (FBS). B, Plot of fibroblast activation signature related genes. C, UMAPs of fibroblast cell clusters at the indicated resolutions. D, heatmap of enriched genes for each fibroblast sub-cluster.

FIG. 19A-FIG. 19B show enriched transcripts of fibroblast cell clusters identified by scRNA-seq from mouse aortas of Ang II treated Cre and TKO mice. A, UMAPs of fibroblast cell cluster (FBS) from aortas of Cre and TKO mice. B, violin plots of representative gene markers, enrich plots, and pathway analysis for the indicated fibroblast cell cluster.

FIG. 20A-FIG. 20B show fibroblast subcluster analyses. A, The percentage of distinct fibroblast clusters. B, Add module score analysis by using canonical fibroblast markers.

FIG. 21A-FIG. 21J show histological analyses and transcriptomic changes in non-stripped aortas of TKO mice with or without anti-IL-9 mAbs. A-B, Representative images of Masson Trichrome staining (MTS) (A) and Sirius Red staining (B) of PBS-treated TKO mice with anti-IL-9 mAb or IgG control. C-D, Representative images of Masson Trichrome staining (MTS) in the heart (C) and kidney (D) (n=6). E, Representative images of Von Kossa staining, and the area of calcium deposition in the aorta (n=5, scale bars=100 μm). F-H, PCA plot of gene expression (F), heatmap and hierarchical clustering (G), and volcano plot of differentially expressed genes (log 2 fold change, >1.5 and FDR, <0.05) (H) in non-stripped aortas from Ang II-treated TKO mice with anti-IL-9 mAb or IgG control (n=6 per group). I, heatmap of top 25 dysregulated genes in non-stripped aortas from Ang II-treated TKO mice with anti-IL-9 mAb or IgG control. J, Overlapping dysregulated genes derived from the dataset of upregulated genes in non-stripped aortas of Ang II-treated Cre and TKO mice and from the dataset of downregulated genes in non-stripped aortas of Ang II-treated TKO mice with anti-IL-9 mAb or IgG controls (top), and the heatmaps of downregulated genes in non-stripped aortas of Ang II-treated TKO mice with anti-IL-9 mAb or IgG controls (bottom). C-E, P values correspond to unpaired two-tailed t tests for normal distributed data.

FIG. 22 shows a graphical abstract.

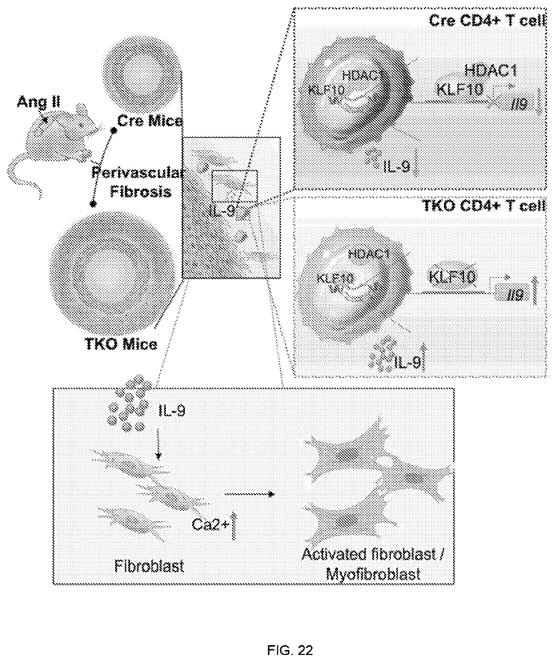

### DETAILED DESCRIPTION

Perivascular fibrosis, characterized by increased amount of connective tissue around vessels, is a hallmark for vascular disease. Angiotensin II (Ang II) contributes to vascular disease and end-organ damage via promoting T-cell activation. Despite recent data suggesting the role of T cells in the progression of perivascular fibrosis, the underlying mechanisms are poorly understood.

The present disclosure is based, at least in part, on the discovery described herein that KLF10 is upregulated in peripheral blood monocytes (PBMCs) of hypertensive patients and in peripheral CD3+ T cells and CD4+ T cells in Ang II-treated mice. As is described herein, administration of Ang II in TKO mice triggered perivascular fibrosis, multi-organ dysfunction in heart, kidney, and aorta, and release of IL-9 from CD4+ T cells. These functional and histopathological differences were independent of blood pressure between Ang II-treated TKO and Cre mice. Without wishing to be bound by any particular theory, mechanistically, in response to Ang II treatment, KLF10 bound to the IL-9 promoter and interacted with HDAC1 to inhibit IL-9 transcription. Ectopic IL-9 activated calcium flux, induced fibroblast activation and differentiation, increased production of collagen and ECM, thereby promoting the progression of perivascular fibrosis and inducing target organ dysfunction. Importantly, IL-9 neutralizing antibodies potently rescued perivascular fibrosis, and ameliorated organ dysfunction in art-accepted animal models such as Ang II treated TKO and C57BL/6 mice. The results described herein demonstrate that the KLF10-IL-9 signaling axis in CD4+ T cells tightly regulate the processes of Ang II-induced pathological perivascular fibrosis and end organ damage and provide new therapeutic opportunities for reducing fibrosis and treatment of hypertensive-associated diseases, for example, using IL-9 antagonists (e.g., anti-IL-9 antibodies) or KLF10 agonists.

Given the above, administration of anti-IL-9 antibodies can be used to treat perivascular fibrosis and other hypertensive diseases or conditions, including without limitation, hypertensive heart disease; isolated systolic, malignant, or resistant hypertension; heart failure with preserved ejection fraction; chronic kidney disease; and coronary heart disease.

Whilst the disclosure has been disclosed in particular embodiments, it will be understood by those skilled in the art that certain substitutions, alterations and/or omissions may be made to the embodiments without departing from the spirit of the disclosure. Accordingly, the foregoing description is meant to be exemplary only, and should not limit the scope of the disclosure. All references, scientific articles, patent publications, and any other documents cited herein are hereby incorporated by reference for the substance of their disclosure.

Below we describe the disclosure as follows:
I. Definitions for understanding the specification;
II. Compositions and methods including therapy; and
III. Examples.

### I. DEFINITIONS

The term “about” as used herein refers to the usual error range for the respective value readily known to the skilled person in this technical field. Reference to “about” a value or parameter herein includes (and describes) embodiments that are directed to that value or parameter per se.

The terms “anti-interleukin-9 (IL-9) antibody,” “an antibody that binds to IL-9,” and “an antibody that specifically binds to IL-9” refer to an antibody that is capable of binding IL-9 with sufficient affinity such that the antibody is useful as a preventative, diagnostic, and/or therapeutic agent in targeting IL-9. In one embodiment, the extent of binding of an anti-IL-9 antibody to an unrelated, non-IL-9 protein is less than about 10% of the binding of the antibody to IL-9 as measured, e.g., by a radioimmunoassay (RIA). In certain embodiments, an antibody that binds to IL-9 has a dissociation constant (K_{D}) of ≤1 μM, <100 nM, <10 nM, <1 nM, <0.1 nM, <0.01 nM, or <0.001 nM (e.g., 10⁻⁸ M or less, e.g., from 10⁻⁸ M to 10⁻¹³ M, e.g., from 10⁻⁹ M to 10⁻¹³ M). In certain embodiments, an antibody that binds to IL-9 has a K_{D} of between about 0.0001 nM and about 100 nM. In some embodiments, the anti-IL-9 antibody is a neutralizing antibody. In some embodiments, the anti-IL-9 antibody is an anti-human IL-9 antibody.

The term “antibody” as used herein in the broadest sense encompasses various antibody structures, including but not limited to monoclonal antibodies, polyclonal antibodies, multispecific antibodies (e.g., bispecific antibodies), and antibody fragments so long as they exhibit the desired antigen-binding activity. An “antibody” can refer, for example, to a glycoprotein comprising at least two heavy chains (HCs) and two light chains (LCs) inter-connected by disulfide bonds, or an antigen binding portion thereof. Each heavy chain is comprised of a heavy chain variable region (VH) and a heavy chain constant region (CH). The heavy chain constant region may be comprised of three domains, CH1, CH2, and/or CH3. Each light chain is comprised of a light chain variable region (VL) and a light chain constant region (CL). The VH and VL regions can be further subdivided into regions of hypervariability, termed “complementarity determining regions” (CDRs), interspersed with regions that are more conserved, termed “framework regions” (FRs). Each VH and VL may be composed, for example, of three CDRs and four FRs, arranged from amino-terminus to carboxyl-terminus in the following order: FR1, CDR1, FR2, CDR2, FR3, CDR3, and FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The terms “full-length antibody,” “intact antibody,” and “whole antibody” are used herein interchangeably to refer to an antibody having a structure substantially similar to a native antibody structure or having heavy chains that contain an Fc region as defined herein.

The term “human antibody” includes antibodies having variable and constant regions (if present) of human germline immunoglobulin sequences. Human antibodies of the disclosure can include amino acid residues not encoded by human germline immunoglobulin sequences (e.g., mutations introduced by random or site-specific mutagenesis in vitro or by somatic mutation in vivo) (see, Lonberg, N. et al. (1994) Nature 368(6474): 856-859); Lonberg, N. (1994) Handbook of Experimental Pharmacology 113:49-101; Lonberg, N. and Huszar, D. (1995) Intern. Rev. Immunol. Vol. 13: 65-93, and Harding, F. and Lonberg, N. (1995) Ann. N.Y. Acad. Sci 764:536-546). However, the term “human antibody” does not include antibodies in which CDR sequences derived from the germline of another mammalian species, such as a mouse, have been grafted onto human framework sequences (i.e., humanized antibodies).

The term “monoclonal antibody,” as used herein, refers to an antibody which displays a single binding specificity and affinity for a particular epitope. Accordingly, the term “human monoclonal antibody,” or “HuMab,” refers to an antibody which displays a single binding specificity, and which has variable and constant regions derived from human germline immunoglobulin sequences. In one embodiment, human monoclonal antibodies are produced by a hybridoma which includes a B cell obtained from a transgenic non-human animal, e.g., a transgenic mouse, having a genome comprising a human heavy chain transgene and a light chain transgene fused to an immortalized cell.

An “antibody fragment” refers to a molecule other than an intact antibody that comprises a portion of an intact antibody that specifically binds to the antigen (e.g., an IL-9 protein) to which the intact antibody binds. Examples of antibody fragments include but are not limited to Fv, Fab, Fab′, Fab′-SH, F(ab′)₂; diabodies; linear antibodies; single-chain antibody molecules (e.g., scFv); and multi-specific antibodies formed from antibody fragments. These antibody fragments are obtained using conventional techniques, and the fragments are screened for utility in the same manner as are intact antibodies. Antibody fragments can be produced by recombinant DNA techniques, or by enzymatic or chemical cleavage of intact immunoglobulins.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Specific illustrative and exemplary embodiments for measuring binding affinity are described below.

The term “EC50,” as used herein, refers to the concentration of an antibody or an antigen-binding portion thereof, which induces a response, either in an in vivo or an in vitro assay, such as neutralization of IL-9 (e.g., blocking IL-9 binding with a binding partner (e.g., an IL-9 receptor (e.g., interleukin-9 receptor (IL9R))) as is described herein, which is 50% of the maximal response (i.e., halfway between the maximal response and the baseline).

The terms “effective amount,” “effective dose,” and “effective dosage” as used herein are defined as an amount sufficient to achieve, or at least partially achieve, the desired effect. The term “therapeutically effective dose” or “therapeutically effective amount” is defined as an amount sufficient to prevent, cure, or at least ameliorate the symptoms of a hypertensive disease or condition, a cardiovascular disease, or chronic kidney disease, or reduce fibrosis (e.g., perivascular fibrosis) and its complications in a patient.

The term “epitope” or “antigenic determinant” refers to a site on an antigen to which an immunoglobulin or antibody specifically binds on IL-9. Epitopes can be formed from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods of determining spatial conformation of epitopes include techniques in the art and those described herein, for example, x-ray crystallography, cryo-electron microscopy, and 2-dimensional nuclear magnetic resonance. See, for example, Epitope Mapping Protocols in Methods in Molecular Biology, Vol. 66, G. E. Morris, Ed. (1996). Epitopes can also be defined by point mutations in the target protein (e.g., IL-9 or a fibrosis-inducing fragment thereof), which affect the binding of the antibody (e.g., monoclonal antibody).

The term “host cell,” as used herein, is intended to refer to a cell into which an expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

An “isolated antibody” is one which has been identified and separated and/or recovered from a component of its natural environment and/or is substantially free of other antibodies having different antigenic specificities (e.g., an isolated antibody that specifically binds to IL-9 is substantially free of antibodies that specifically bind antigens other than IL-9). Contaminant components of its natural environment are materials which would interfere with diagnostic or therapeutic uses for the antibody, and may include enzymes, hormones, and other proteinaceous or non-proteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie™ blue or, preferably, silver stain. Isolated antibody includes the antibody in situ within recombinant cells since at least one component of the antibody's natural environment will not be present. Similarly, isolated antibody includes the antibody in medium around recombinant cells. Ordinarily, however, isolated antibody will be prepared by at least one purification step.

The term “nucleic acid molecule,” as used herein, is intended to include DNA molecules and RNA molecules. A nucleic acid molecule may be single-stranded or double-stranded, but preferably is double-stranded DNA.

The term “isolated nucleic acid,” as used herein in reference to nucleic acids molecules encoding antibodies or antibody portions (e.g., VH, VL, CDRs) that bind to IL-9, is intended to refer to a nucleic acid molecule in which the nucleotide sequences encoding the antibody or antibody portion are free of other nucleotide sequences encoding antibodies that bind antigens other than IL-9, which other sequences may naturally flank the nucleic acid in human genomic DNA.

As used herein, “percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, or MEGALIGN™ (DNASTAR) software. Those skilled in the art can determine appropriate parameters for measuring alignment, including any algorithms needed to achieve maximal alignment over the full length of the sequences being compared.

The term “pharmaceutical composition” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, the terms “specific binding,” “selective binding,” “selectively binds,” and “specifically binds,” refer to antibody binding to an epitope on a predetermined antigen. Typically, the antibody binds with an affinity (K_{D}) of approximately less than 10⁻⁷ M, such as approximately less than 10⁻⁸ M, 10⁻⁹ M or 10⁻¹⁰ M or even lower when determined by surface plasmon resonance (SPR) technology in a BIACORE 3000 instrument, which can be performed, for example, using recombinant IL-9 as the analyte and the antibody as the ligand. In some embodiments, binding by the antibody to the predetermined antigen is with an affinity that is at least two-fold greater than its affinity for binding to a non-specific antigen (e.g., BSA, casein) other than the predetermined antigen or a closely related antigen. The phrases “an antibody recognizing an antigen” and “an antibody specific for an antigen” are used interchangeably herein with the term “an antibody which binds specifically to an antigen.”

A “subject,” a “patient,” or an “individual” is typically a human such as an adult, a child, or an infant.

As used herein, “administering” is meant a method of giving a dosage of a compound (e.g., an anti-IL-9 antibody or a KLF10 agonist) or a composition (e.g., a pharmaceutical composition, e.g., a pharmaceutical composition including an anti-IL-9 antibody or a KLF10 agonist) to a subject. The compositions utilized in the methods described herein can be administered, for example, parenterally, intramuscularly, intravenously, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions. The administration may be local or systemic. The method of administration can vary depending on various factors (e.g., the compound or composition being administered, and the severity of the disease being treated).

As used herein, the term “vector” is meant to include, but is not limited to, a nucleic acid molecule (e.g., a nucleic acid molecule that is capable of transporting another nucleic acid to which it has been linked), a virus (e.g., a lentivirus, an adenovirus, or a recombinant adeno-associated virus (rAAV)), cationic lipid (e.g., liposome), cationic polymer (e.g., polysome), virosome, nanoparticle, or dendrimer. Accordingly, one type of vector is a viral vector, wherein additional DNA segments (e.g., a transgene, e.g., a transgene encoding KLF10) may be ligated into the viral genome, and the viral vector may then be administered (e.g., by electroporation, e.g., electroporation into muscle tissue) to the subject to allow for transgene expression in a manner analogous to gene therapy. Another type of vector is a “plasmid,” which refers to a circular double stranded DNA loop into which additional DNA segments may be ligated. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids.

The terms “interleukin 9” and “IL-9” as used herein, refer to any native IL-9 from any vertebrate source, including mammals such as primates (e.g., humans) and rodents (e.g., mice and rats), unless otherwise indicated. The term encompasses “full-length,” unprocessed IL-9 as well as any form of IL-9 that results from processing in the cell. The term also encompasses naturally occurring variants of IL-9, e.g., splice variants or allelic variants. The nucleic acid sequence of an exemplary wild-type human IL-9 gene is provided, e.g., in NCBI Reference Sequence: NC_000005.10:c135895841-135892246. The nucleic acid sequence of an exemplary human IL-9 is set forth in SEQ ID NO:41. The nucleic acid sequence of an exemplary human IL-9 cDNA (GenBank: BC066286.1) is set forth in SEQ ID NO:42. The amino acid sequence of an exemplary wild-type human IL-9 protein is provided, e.g., in NCBI Reference Sequence: NP_000581.1. The amino acid sequence of an exemplary protein encoded by human IL-9 is set forth in SEQ ID NO:43. IL-9 belongs to the interleukin group of cytokines. IL-9 is a cell signaling molecule that is secreted by different cell types such as Th2, Th9, Th17, mast cells, and NKT cells, among others. IL-9 has a variety of functions such as promoting mast cell growth and function, stimulation of cell proliferation, prevention of apoptosis, and regulation of hematopoietic cells. SEQ ID NO:41 is shown below:

AAGCGAGCTCCAGTCCGCTGTCAAGATGCTTCTGGCCATGGTCCT
TACCTCTGCCCTGCTCCTGTGCTCCGTGGCAGGCCAGGGGTGTCC
AACCTTGGGGGGGATCCTGGACATCAACTTCCTCATCAACAAGAT
GCAGGTAGGCTGCAGGGGGAGCCCATGGGAAAGACAGCTACTGAC
AAAGTGAAATATGTATGAGGATGAAAAAACTCGGGGCTGACTAAA
GGTTCTTATCTCTCTATCTACTTTAGGAAGATCCAGCTTCCAAGT
GCCACTGCAGTGCTAATGTGAGTGAATGCTCTTTAAGAACTTTCC
AAATTAATTTTAATTTTCACATCTGGAATCTTCACTCTGAAATTT
CCCTTGCAGGTGACCAGTTGTCTCTGTTTGGGCATTCCCTCTGTA
AGTATAGTGAAATAACATAATGTTGACCTTGGATTTTTTTGGTTT
GTTTTTAAGTAAAAATAAGTTGCTTTATTTAATATTTAATGTTAT
ACATTGTTGCTTAATTTAATTGTTACAGATTAGTATTCCCTGTTA
AAACCACATTGTTACAAATTATTCCCTTTTAAAACTACGATCTTG
AAATCCTATATTATGAACATTTCTTTGTATTTAATTAACTTTATG
CCTCTTGAGAAGTTTGAACACTTTTCAACATTAAAAAAAGAATCC
TGAATATCTTTTTAGATAGGTGGCCATGTGCACAATTAAATAAAA
CTGGAACTAAGGATATAATAATTGCTGTAGCTCATATCATATTGC
TTTCTAACTCATTTACTGATAACTCTAGAGTTGTGAAACAATGTA
AATAAAATGACAACTCCTTATCTTTCATCTGTCATGAATGATCTA
TGCGCTATACCTCCCCCTCCCTGCCTCCTCCCTTCCTCCCCACCA
CCCTGTTGTCTGTCTAGCTGATTAGAGTGACTGTTGGTTTGAATG
CTGCCCTCTGGGCAGGTAGAGGATCTGAGGTTGTGAGTGGAAGGA
GGGCTTCCAGAGGGCCACTGCCCACTACGGCAGGAAGGATGGGTG
GCAGGAAAGTTCTGATTCCTAATTCAAACTCCTGGTTAGGGTGAG
GAGGAGGCACTTCTCCAAGGTGCAGTGCTTTATTCTTTCTCATGC
AAGGCCTGGGAGAATCTGAAGAATCTGAGCTTCTTGCCCTGGCTA
GGGTAAGACATCGCACCCATCGCGGTCCATCCATTAGATGAGAAG
AGGATAGAGTGCCTTCTGGGCAGGAACCAGGCAGACAGCACAGCC
CCTGTCCCTTGGAGTACCGTCCATGTTTTTAGCTGCTGCTGAAAT
ACCAGCTGCATTCAATTGTCACATCCCATTAGCTGGTGTGAAAAG
GCTTTTCCTCACTCTGCACTTTCAGACTTACAAGCCTTGAAGCCG
GGAAGCACCCGTTGAAAAGAACATTCAGAGCCGACTATTTCAGGG
CCCAGAGCCCTCATGTTTCCTGGATGTAACATACAGGAAGTCTCC
TCCAGGGGATGTCACTGTGGAAAAATGGCATCCCCTTTAAATACG
GGAGATCACTTCCTACATTGGCAAGGGACCTGTCTAAAAATAATG
CAAGTTTGAGTAATGGTGATTAAATAAAAATCATCTCTATTATAT
TGCTCTTTGTGATATATTTCCAAAGCTGTCCTCAGAATATTTCTT
TGAATAAATCCTTACTATTTACCAGGACAACTGCACCAGACCATG
CTTCAGTGAGAGACTGTCTCAGATGACCAATACCACCATGCAAAC
AAGATACCCACTGATTTTCAGTCGGGTGAAAAAATCAGTTGAAGT
ACTAAAGAACAACAAGTGTCCAGTAAGTTTGTTTTCATATGTGAT
ATGTTCCTGTTGGTGATTTCTATGTGAATGGTGATGCCAACCCTG
TTTGAACACAAAAGGATGATAAAGTTGGAATTGGTAGTTCAAGGT
TGATAAAAGACATCTAAGAATTTTAATCAGAAGTAATATAATTAA
AGTGAGATCCACTGAAACAATAGAATTAAAGTGAGATAGATCATT
GTTCCTGACGAGGCCATTTACTTCTCTCTACTATGGAATAATGAA
AGAATCCTTTCTGAGTGTAATTAGAAGCTACAATCTAGAGAATCA
GGGATGTAGCTCACATAATACTAAATTATCCTAGAGATTCAATGT
ACTAACTGAATGGATGTTGTTAACAGGGATTTTTTTTTCCTGTTG
GTTAAGGAGGTTTTGTTTTGTTTTGGAGACAGAGTCTTGCTCTGT
TGCCCAGGCTGGAGTGCAGTGGTGCCATCTGAGCTCACTGCAGCC
TCTGCCTCCCGGGTTCAAGTGATTATCCTGCCTCAGCCTCCCGAG
TAGCTGGCATTACAGGTGCGTGCCACCATGCCTGGCTAATTTTTG
TATTTTTAATAGAGATGGGGTTTCACCATGTTGGCCAGGTTGCTC
TCCAACTCCTGAACTCAAGTGATTTGCCCGCCTTGACCTCCCAAA
GTGCTGGGATGACAGGTGTGAGCCACCATGCCTGGCCTGCATTAA
GGAGGTATTTAAAGGGCAATGCACCCAGGTCAAGGTGGAAGCTTG
CTACTCATCCTGAATGCCCATCCACACATTCTTTTCTTCAGCATA
TACCCTAGTCCCTGACAGCAGACTGGGATGGCAAGTTGGGTAGAG
GTGACCTCCCTCTGTTTTTTGGGTATTAGCATCTCCACACAAGAT
CCTAGAAGGCTGAAAGCCCTGAGCTCAGCTGTTTAGCTGCATGCG
TTTCTACCATCAATGGCATCTAGTTCTAAGTGCTTAATATATGCT
GTCTCACTGAATAAATACATACCTTAGGGACAATTATTCAATTTA
TTACTCTCAGTGAGGTTAACTAATTTGCCTAAGGCTGCATATTTG
ATAAGTGGCAGAGCTGAGATTTGAACTCAGGCCTATATGACCTCA
GAGCCCCACTCTTAGCCATTGTACTGTCAAATGACCTTGGAAAGA
CAACCTAAAAGGATAATGATACAATTTTAGGCCTCAAAGAGTCCC
CAGAAAAGGCTTTCTCTAATGCAGAGATTTAGGGCCACTTAATAG
GGGTGTGTGTGTGTGTGTGTGTGTGTGTGTGTGTGTGTGTGTGTG
TGTAAAGACCCCTGAAATCCAATTTGAGGTCAACCACCTATGCTG
TCTTTACACCACATGAGCTAGCCTGGACCTGCCCACCTATTTGCT
CTGTGTCTCAAGCCACTTCCCTTCCCATCCCCACAATCCTCACCA
CCGACTCTGGCTCTTGGCAGGTAGGCTTCTGGGGCTGCTTGGCTC
TACATCATTTGAGTCACTCTGTCCTTATCAACTTTCATCCCCACA
GTATTTTTCCTGTGAACAGCCATGCAACCAAACCACGGCAGGCAA
CGCGCTGACATTTCTGAAGAGTCTTCTGGAAATTTTCCAGAAAGA
AAAGATGAGAGGGATGAGAGGCAAGATATGAAGATGAAATATTAT
TTATCCTATTTATTAAATTTAAAAAGCTTTCTCTTTAAGTTGCTA
CAATTTAAAAATCAAGTAAGCTACTCTAAATCAGTATCAGTTGTG
ATTATTTGTTTAACATTGTATGTCTTTATTTTGAAATAAAT

SEQ ID NO:42 is shown below:

CCGCTGTCAAGATGCTTCTGGCCATGGTCCTTACCTCTGCCCTGC
TCCTGTGCTCCGTGGCAGGCCAGGGGTGTCCAACCTTGGCGGGGA
TCCTGGACATCAACTTCCTCATCAACAAGATGCAGGAAGATCCAG
CTTCCAAGTGCCACTGCAGTGCTAATGTGACCAGTTGTCTCTGTT
TGGGCATTCCCTCTGACAACTGCACCAGACCATGCTTCAGTGAGA
GACTGTCTCAGATGACCAATACCACCATGCAAACAAGATACCCAC
TGATTTTCAGTCGGGTGAAAAAATCAGTTGAAGTACTAAAGAACA
ACAAGTGTCCATATTTTTCCTGTGAACAGCCATGCAACCAAACCA
CGGCAGGCAACGCGCTGACATTTCTGAAGAGTCTTCTGGAAATTT
TCCAGAAAGAAAAGATGAGAGGGATGAGAGGCAAGATATGAAGAT
GAAA

SEQ ID NO:43 is shown below:

MLLAMVLTSALLLCSVAGQGCPTLAGILDINFLINKMQEDPASKC
HCSANVTSCLCLGIPSDNCTRPCFSERLSQMTNTTMQTRYPLIFS
RVKKSVEVLKNNKCPYFSCEQPCNQTTAGNALTFLKSLLEIFQKE
KMRGMRGKI

The terms “Kruppel-like factor 10”, “KLF transcription factor 10”, and “KLF10” as used herein, refer to any native KLF-10 from any vertebrate source, including mammals such as primates (e.g., humans) and rodents (e.g., mice and rats), unless otherwise indicated. The term encompasses “full-length,” unprocessed KLF-10 as well as any form of KLF-10 that results from processing in the cell. The term also encompasses naturally occurring variants of KLF10, e.g., splice variants or allelic variants. The nucleic acid sequence of an exemplary wild-type human KLF10 gene is provided, e.g., in NCBI Reference Sequence: NC_000008.11:c102655725-102648784. The nucleic acid sequence of an exemplary human KLF10 is set forth in SEQ ID NO:44. The nucleic acid sequence of an exemplary human KLF10 cDNA (GenBank: BC095399.1) is set forth in SEQ ID NO:45. An exemplary wild-type human KLF10 protein is provided, e.g., in NCBI Reference Sequence: NP_001027453.1. The amino acid sequence of an exemplary protein encoded by human KLF10 is set forth in SEQ ID NO:46. KLF10 is a transcription factor that is encoded by the KLF10 gene that affects transforming growth factor beta (TGF-β) signaling. In general, KLF10 functions as a transcriptional repressor.

As used herein, the term “agonist” is a molecule that mimics the effects of an endogenous protein (e.g., KLF10) and produces a biological response that is the same or similar as the biological response produced by the endogenous protein. In some embodiments, the KLF10 agonist includes a small molecule agonist, recombinant KLF10, or a vector (e.g., a viral vector (e.g., adeno-associated viral vector)) including a nucleic acid encoding KLF10.

### I1. COMPOSITIONS AND METHODS

### A. Anti-IL-9 Antibodies

The disclosure provides antibodies (e.g., any of the antibodies described herein) that bind to IL-9. For example, the disclosure provides isolated antibodies (e.g., any of the antibodies described herein) that bind to IL-9.

Accordingly, in one aspect, the disclosure an antibody that specifically binds to IL-9.

Antibodies of the disclosure may, for example, be monoclonal, human, humanized, or chimeric. The antibodies can be full-length antibodies, or antibody fragments thereof (e.g., an antibody fragment that binds IL-9). The antibody fragment may be selected from the group consisting of Fab, Fab′-SH, Fv, scFv, and (Fab′)₂ fragments. In some instances, the antibody is an IgG antibody (e.g., an IgG1 antibody or an IgG4 antibody). An antibody of the disclosure may have a half-life of 3 days (e.g., ≥1 week, e.g., ≥2 weeks, e.g., ≥1 month, e.g., ≥2 months, e.g., ≥3 months, e.g., ≥4 months, e.g., ≥5 months, e.g., ≥6 months). Methods of making anti-IL-9 antibodies are well known in the art. The anti-IL-9 antibodies are therapeutic agents. In some embodiments, the anti-IL-9 antibody is an anti-human IL-9 antibody.

Any suitable anti-IL-9 antibody may be used. Such antibodies are known in the art and can be obtained from public sources, for example from BioLegend, San Diego, CA or from Creative Biolabs (enokizumab), Shirley, NY. In one example, the anti-IL-9 antibody is BE0181 (BIO X CELL, NH, USA). In another example, the anti-IL-9 antibody is clone D9302C12, BD Pharmingen, San Diego, CA). In another example, the anti-IL-9 antibody is clone MH9A3 (Creative Biolabs). In some embodiments, the anti-IL-9 antibody includes enokizumab. In some embodiments, the heavy chain of enokizumab has the amino acid sequence of SEQ ID NO:47. SEQ ID NO:47 is shown below:

QVQLVQSGAEVKKPGSSVKVSCKASGGTFSYYWIEWVRQAPGQGL
EWMGEILPGSGTTNPNEKFKGRVTITADESTSTAYMELSSLRSED
TAVYYCARADYYGSDYVKFDYWGQGTLVTVSSASTKGPSVFPLAP
SSKSTSGGTAALGCLVKDYFPEPVTVSWNSGALTSGVHTFPAVLQ
SSGLYSLSSVVTVPSSSLGTQTYICNVNHKPSNTKVDKRVEPKSC
DKTHTCPPCPAPELLGGPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
EEMTKNQVSLTCLVKGFYPSDIAVEWESNGQPENNYKTTPPVLDS
DGSFFLYSKLTVDKSRWQQGNVFSCSVMHEALHNHYTQKSLSLSP
GK

In some embodiments, the light chain of enokizumab has the amino acid sequence of SEQ ID NO:48. SEQ ID NO:48 is shown below:

DIQMTQSPSSLSASVGDRVTITCKASQHVITHVTWYQQKPGKAPK
LLIYGTSYSYSGVPSRFSGSGSGTDFTLTISSLQPEDFATYYCQQ
FYEYPLTFGGGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC

Enokizumab is also described in WHO Drug Information (International Nonproprietary Names for Pharmaceutical Substances (Proposed INN) List 104, Vol. 24, No. 4, 2010, pp. 368-369.

In some embodiments, the anti-IL-9 antibody includes one or more heavy chain complementarity determining regions (CDRs), i.e., CDR-H1, CDR-H2, and/or CDR-H3 of enokizumab, and/or one or more light chain CDRs, i.e., CDR-L1, CDR-L2, and/or CDR-L3 of enokizumab. For example, in some embodiments, the anti-IL-9 antibody includes one or more, or all, of the following: (a) a CDR-H1 comprising the amino acid sequence of YYWIE (SEQ ID NO:49); (b) a CDR-H2 comprising the amino acid sequence of EILPGSGTTNPNEKFKG (SEQ ID NO:50); (c) a CDR-H3 comprising the amino acid sequence of ADYYGSDYVKFDY (SEQ ID NO:51); (d) a CDR-L1 comprising the amino acid sequence of KASQHVITHVT (SEQ ID NO:52); (e) a CDR-L2 comprising the amino acid sequence of GTSYSYS (SEQ ID NO:53); and/or (f) a CDR-L3 comprising the amino acid sequence of QQFYEYPLT (SEQ ID NO:54). In some embodiments, the foregoing CDR sequences are according to the Kabat numbering scheme, but other numbering schemes (e.g., IMGT or Chothia) can be used.

The therapeutic agents (e.g., anti-IL-9 antibodies) described herein may further be modified (e.g., chemically modified). Modifications may be designed to facilitate manipulation or purification of the molecule, to increase solubility of the molecule, to facilitate administration, targeting to the desired location, to increase or decrease half-life. A number of such modifications are known in the art and can be applied by the skilled practitioner.

In a further aspect, an anti-IL-9 antibody according to any of the above embodiments may incorporate any of the features, singly or in combination, as described in Sections 1-5 below.

### 1. Antibody Affinity

In certain embodiments, an antibody provided herein may have a dissociation constant (K_{D}) of ≤10 μM, ≤1 μM, ≤100 nM, ≤10 nM, ≤1 nM, ≤0.1 nM, ≤0.01 nM, or ≤0.001 nM.

In one embodiment, K_{D} is measured by a radiolabeled antigen binding assay (RIA). In one embodiment, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 μM or 26 μM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed, and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, K_{D} is measured using a BIACORE® surface plasmon resonance assay. For example, an assay using a BIACORE®-3000 (BIAcore, Inc., Piscataway, NJ) is performed at 25° C. with immobilized antigen CM5 chips at ˜10 response units (RU). In one embodiment, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (K_{D}) is calculated as the ratio k_{off}/kₒₙ. See, for example, Chen et al., J. Mol. Biol. 293:865-881 (1999). If the on-rate exceeds 10⁶M⁻¹s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophotometer (Aviv Instruments) or a 8000-series SLM-AMINCO™ spectrophotometer (ThermoSpectronic) with a stirred cuvette.

### 2. Antibody Fragments

In certain embodiments, an antibody provided herein is an antibody fragment. Antibody fragments include, but are not limited to, Fab, Fab′, Fab′-SH, F(ab′)₂, Fv, and scFv fragments, which are known in the art. Also included are diabodies, which have two antigen-binding sites that may be bivalent or bispecific, as is known in the art. Triabodies and tetrabodies are also known. Single-domain antibodies are also antibody fragments comprising all or a portion of the heavy chain variable domain or all or a portion of the light chain variable domain of an antibody. In certain embodiments, a single-domain antibody is a human single-domain antibody.

Antibody fragments can be made by various techniques, including but not limited to proteolytic digestion of an intact antibody as well as production by recombinant host cells (e.g., E. coli or phage), as described herein.

### 3. Chimeric and Humanized Antibodies

In certain embodiments, an antibody provided herein is a chimeric antibody. In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Human framework regions that may be used for humanization include but are not limited to framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### 4. Human Antibodies

In certain embodiments, an antibody provided herein is a human antibody (e.g., a human monoclonal antibody (HuMab), e.g., an anti-IL-9 HuMab). Human antibodies can be produced using various techniques known in the art.

In some instances, human antibodies may be prepared by administering an immunogen (e.g., IL-9) to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extra chromosomally or integrated randomly into the animal's chromosomes. In such transgenic mice, the endogenous immunoglobulin loci have generally been inactivated. Human variable regions from intact antibodies generated by such animals may be further modified, for example, by combining with a different human constant region.

In some instances, human antibodies can also be made by hybridoma-based methods, as described in further detail below. Human myeloma and mouse-human heteromyeloma cell lines for the production of human monoclonal antibodies have been described.

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

### 5. Antibody Variants

In certain embodiments, amino acid sequence variants of the anti-IL-9 antibodies are contemplated.

For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, for example, antigen-binding.

In certain embodiments, antibody variants having one or more amino acid substitutions are provided. Sites of interest for substitutional mutagenesis include the CDRs and FRs. Conservative substitutions are shown in Table 1 under the heading of “preferred substitutions.” More substantial changes are provided in Table 1 under the heading of “exemplary substitutions,” and as further described below in reference to amino acid side chain classes. Amino acid substitutions may be introduced into an antibody of interest and the products screened for a desired activity, for example, retained/improved antigen binding, decreased immunogenicity, or improved ADCC or CDC.

TABLE 1
Exemplary and Preferred Amino Acid Substitutions

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generality, the resulting variant(s) selected for further study will have modifications (e.g., improvements) in certain biological properties (e.g., increased affinity, reduced immunogenicity) relative to the parent antibody and/or will have substantiality retained certain biological properties of the parent antibody. An exemplary substitutional variant is an affinity matured antibody, which may be conveniently generated, e.g., using phage display-based affinity maturation techniques such as those described herein. Briefly, one or more CDR residues are mutated, and the variant antibodies displayed on phage and screened for a particular biological activity (e.g., binding affinity).

Alterations (e.g., substitutions) may be made in CDRs, for example, to improve antibody affinity. Such alterations may be made in CDR “hotspots,” i.e., residues encoded by codons that undergo mutation at high frequency during the somatic maturation process, and/or residues that contact antigen, with the resulting variant VH or VL being tested for binding affinity. Affinity maturation by constructing and reselecting from secondary libraries is known in the art. In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. Such alterations may, for example, be outside of antigen contacting residues in the CDRs. In certain embodiments of the variant VH and VL sequences provided above, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intra-sequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

In certain embodiments, alterations may be made to the Fc region of an antibody. These alterations can be made alone, or in addition to, alterations to one or more of the antibody variable domains (i.e., VH or VL regions) or regions thereof (e.g., one or more CDRs or FRs). The alterations to the Fc region may result in reduced antibody effector functions (e.g., complement-dependent cytotoxicity (CDC))

In certain instances, the disclosure contemplates an antibody, e.g., antibody variant that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity) but retains FcRn binding ability. The primary cells for mediating ADCC, NK cells, express FcγRIII only, whereas monocytes express FcγRI, FcγRII, and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CYTOTOX 96© non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al. J. Immunol. Methods 202:163 (1996); Cragg, M. S. et al. Blood. 101:1045-1052 (2003); and Cragg, M. S. and M. J. Glennie Blood. 103:2738-2743 (2004)). FcRn binding and in vivo clearance/half-life determinations can also be performed using methods known in the art (see, e.g., Petkova, S. B. et al. Int'l. Immunol. 18(12):1759-1769 (2006)).

Antibodies with reduced effector function include those with substitution of one or more of Fc region residues 238, 265, 269, 270, 297, 327 and 329 (U.S. Pat. Nos. 6,737,056 and 8,219,149). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. Nos. 7,332,581 and 8,219,149).

In certain instances, the proline at position 329 of a wild-type human Fc region in the antibody is substituted with glycine or arginine or an amino acid residue large enough to destroy the proline sandwich within the Fc/Fcγ receptor interface that is formed between the proline 329 of the Fc and tryptophan residues Trp 87 and Trp 110 of FcγRIII (Sondermann et al.: Nature 406, 267-273 (20 Jul. 2000)). In certain instances, the antibody comprises at least one further amino acid substitution. In one instance, the further amino acid substitution is S228P, E233P, L234A, L235A, L235E, N297A, N297D, or P331S, and still in another instance the at least one further amino acid substitution is L234A and L235A of the human IgG1 Fc region or S228P and L235E of the human IgG4 Fc region (see e.g., US 2012/0251531), and still in another instance the at least one further amino acid substitution is L234A and L235A and P329G of the human IgG1 Fc region.

In certain embodiments, alterations of the amino acid sequences of the Fc region of the antibody may alter the half-life of the antibody in the host. Certain mutations that alter binding to the neonatal Fc receptor (FcRn) may extend half-life of antibodies in serum. For example, antibodies that have tyrosine in heavy chain position 252, threonine in position 254, and glutamic acid in position 256 of the heavy chain can have dramatically extended half-life in serum (see, e.g., U.S. Pat. No. 7,083,784).

In certain instances, antibodies of the disclosure can be altered to increase or decrease the extent to which the antibody is glycosylated. Addition or deletion of glycosylation sites to an antibody of the disclosure may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some instances, modifications of the oligosaccharide in an antibody of the disclosure are made in order to create antibody variants with certain improved properties.

In certain instances, it is desirable to create cysteine engineered anti-IL-9 antibodies, e.g., “thioMAbs,” in which one or more residues of an antibody are substituted with cysteine residues. In particular instances, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain instances, any one or more of the following residues are substituted with cysteine: V205 (Kabat numbering) of the light chain; A118 (EU numbering) of the heavy chain; and S400 (EU numbering) of the heavy chain Fc region. Cysteine engineered antibodies may be generated as described, for example, in U.S. Pat. No. 7,521,541.

In certain instances, an antibody of the disclosure provided herein are further modified to contain additional non-proteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

### B. Characterization of Anti-IL-9 Antibodies

Sequence information for antibodies described herein can be ascertained using sequencing techniques which are well known in the art.

Similarly, affinity of the antibodies for IL-9 can also be assessed using standard techniques. For example, Biacore 3000 can be used to determine the affinity of such antibodies. Antibodies are captured on the surface of a Biacore chip (GE healthcare), for example, via amine coupling (Sensor Chip CM5). The captured antibodies can be exposed to various concentrations of IL-9 in solution, and the Kₒₙ and K_{off} for an affinity (K_{D}) can be calculated, for example, by BIAevaluation software.

Antibodies can also be characterized for binding to IL-9 using a variety of known techniques, such as enzyme-linked immunosorbent assay (ELISA), Western blot, biolayer interferometry (BLI), and the like. Generally, the antibodies are initially characterized by ELISA. Briefly, microtiter plates can be coated with purified IL-9 in PBS, and then blocked with irrelevant proteins such as bovine serum albumin (BSA) diluted in PBS. Dilutions of plasma are added to each well and incubated for 1-2 hours at 37° C. The plates are washed with PBS/TWEEN® 20 and then incubated with a goat-anti-human IgG Fc-specific polyclonal reagent conjugated to alkaline phosphatase for 1 hour at 37° C. After washing, the plates are developed with ABTS substrate, and analyzed at OD of 405. In some examples, the ELISA may be an IMMUNOCAP™ ELISA assay.

In other instances, competition assays may be used to identify an antibody that competes with an anti-IL-9 antibody for binding to IL-9. In certain embodiments, such a competing antibody binds to the same epitope (e.g., a linear or a conformational epitope) that is bound by an anti-IL-9 antibody of the disclosure. Detailed exemplary methods for mapping an epitope to which an antibody binds are provided in Morris (1996) “Epitope Mapping Protocols,” in Methods in Molecular Biology vol. 66 (Humana Press, Totowa, NJ).

In an exemplary competition assay, immobilized IL-9 is incubated in a solution comprising a first labeled antibody that binds to IL-9 and a second unlabeled antibody that is being tested for its ability to compete with the first antibody for binding to IL-9. The second antibody may be present in a hybridoma supernatant. As a control, immobilized IL-9 is incubated in a solution comprising the first labeled antibody but not the second unlabeled antibody. After incubation under conditions permissive for binding of the first antibody to IL-9, excess unbound antibody is removed, and the amount of label associated with immobilized IL-9 is measured. If the amount of label associated with immobilized IL-9 is substantially reduced in the test sample relative to the control sample, then that indicates that the second antibody is competing with the first antibody for binding to IL-9.

### C. KLF10 Agonists

Provided herein are KLF10 agonists. Any suitable KLF10 agonists may be used. In some embodiments, the KLF10 agonist includes a small molecule agonist, recombinant KLF10, or a vector (e.g., a viral vector (e.g., adeno-associated viral vector)) including a nucleic acid encoding KLF10.

In some embodiments, the KLF10gene has the nucleic acid sequence of SEQ ID NO:44. SEQ ID NO:44 is shown below:

GCAGACGGCGCTGAGCGCGGGGGGGGGGGGAGCGGCGTCGAGTGT
CTCCGTGCGCCCGTCTGTGGCCAAGCAGCCAGCAGCCTAGCAGCC
AGTCAGCTTGCCGCCGGCGGCCAAGCAGCCAACCATGCTCAACTT
CGGTGCCTCTCTCCAGCAGACTGCGGTAAGTCATTTGGGGATGCC
CCTGTGCTTCCTCGCCTGGTCTTGTCTGGGGGGCCAAAGGGGGCG
CGAACCCCGAGCCCCGGACATCAGCCATGCCTGAGAATTGGGGCT
GCAGCGGAGTCGTGGGGAAGGAAAGGGCTTCCTGCCTGCAGACTA
TGGGCATTAGTGAGGGCGTGTGTGTTCGGGAGGGGGTCGAACCAG
GGGGCTGGGATQTTCAGACAGGGACAGGGGTCTTGCTCTAGATGT
ACTGAGGGGAAGGGACAACTCCGCATGGAGACCCGAGAGGGCTGG
TGAGGAGGAGGATGACGAGCGGGGGAGGAGTGGGGAGGGGGCCGT
TGCCCTGCAGCGAGGTTGGGGTACAGTGTGGAACGAGAGTCGCGG
GAGGGACAAGGGGGCTACCCTCACCTGGTACCGAGGAGAGATGCC
CGGTCTGGCTCAGGAACGCGAGCCAGGGGAGGAAGGGGCTGCTCG
CAAGCACTCGGGAGGCGGGGTGTCCACAGGAGAGGCGAGTGGGCT
ACCCATCCTGGGGGTCTGAACCCACGCTTGGAAGACGGGGGCTGC
TCAGCAGATAGCTCGGGGCTGGGGGAAGGGGCGCCTGTCGTCGGA
GCGGGGGGATGACACGAGGGCCCGCCGACAGCCCTGGGGGACGAG
TTGCTCCCCCAGACGTCTGGAGGGGGACTCTTACCCCAGGGGCAG
GTGGCCGCCTTCTGACGACTCCTTGGAAGAGGGGGGAGCCGCCTC
TGGGGCAGGAGGGTGGGAAGCGGGACCCAGCAGCTGGGGGGGGAA
GGTGGGGGGGGGAGCTGGGCCGCGAGACGCGGGGGCCGCGCGTCT
GGAGCCGGGGCCTGTGGGGGCTGCACGTCTGGGAACCCGTGGAGA
CGCGCGGGGGGAGGGGCCTGGAGCTGCCGGAAGCCACGGGGAGGG
GGCGCCGCGTCTACAGGGAGAGACGGGGTGGTCTGAGGTCCCGTG
GGCCGCGTGGGGGGAAGTGCCGAGGGCTCGGCCGGGTTCCCGGGG
GCTGCCGGGGGGAGGAGGGGGGCGGCCGGGTTTCCGCGGGGACCT
GGCTCCTCCTCCCGCATCTGAGGCGGAGGCCGGGGGTGGGCCGGG
AGCGGGTGCGCCCGGGGAGCTTCTCGTCCCCGAGTGACCCAGCCC
CGGCCTCCGCCAGGGGGGGGAGCGAGGCCCGGCCCCGCTGTCGGT
TCCTAAGGGAACGGCGGCTCGCGGGGAGCTGGAAGGGGGGGGCGC
CTCCCGGAGCCTGGCGCCCGCAGCCCCGCCCCTCCCCGCGGGCCC
GGCGCGCACGGCCGGGCGGTTGGCGGCGGCGCCCGCGGCCCCTCC
CCCGGGTCGGGCGCGGGGGGGGGCCCGCAAGCTCAGGAAGTAGGG
GAAAGGTGACGGCGCGGCAATTCCCAGTTCACGTTTCCTGCGCCT
CCCGGAGCAGCCGCTGATCACGCTTTGTTCACATGCCTCGCCCCC
TCCTCGCCCACGCCCCCGCCCCGGGCCGCACAGCCAATCCGGGCT
CGGGCCGCCGAGCCGGCCGGCCAATGGGGGGGGCAGGGGCGCGGG
GACGTGCGAGCGGCGAGGAATGTTCCCAGTGACTCACCCGTGAGC
CTCATTGAGGTTAGGGGGGCCCCATCCGTCGGTCTCCGCCAGCTA
GGATCTCCCCCGCCGTCCGCCCCCGCCCCTCGTCTGCCTCCCCCG
CCCGCGCCTCCGCCCGCGCTTCCCTTTCCTGCCTCGCGCCCCACT
CCCTTTCCTCCCCTCCCTGTTTCCCTTCCTGTCCTTCCCAGCTCA
CGCTCTCTTTCCCTGCCGCCTGCCTTTCTTTTTTCCTTTTTTTGC
ATTGGCGTCTTGGGGCTGTTACACACACGCGCGCTGTCCATTGCA
GCTTACATAAAGGCGGGCGCGATTATGCAATTATATTGTTAGCGA
TATTTCAAGAGCAATGGCTCGTTTTCTTAGGATTTCAACACGAAG
GCATCATGCATTTTTGAAAAACTAGTATTGAGAATAATACCTTGC
AACGTAAAGAATGTTTTTTGGTATTTTTACACAATCTCTACTTTG
ACCAAACGAGTCTGGACAGTTTTCTTTTAATGGAAAATAGGAGAA
ATGGTGAGTAGTACCATTTTTTTTTAGCGTAGTTCAAAGAACTTT
ACTATAAAACATTAGGTAGTTCTACAAATAAGTGCAAAACCTGTG
CTGTTATTTTGTCATCAAGCAGTTCTTTAAGGAATTAAAATAGAT
TCTACTTTGATTATGCTGACTTTTTAAAGTCGGTTTTTGCAAATT
CCAAGTAGAATATTAAATTTTTTGATTATCCAGTTTTTAGTAATA
TTGCATTAGCTATGTTGTAGTTATGTATGGCAAACCATTGTGTCA
AATAGATTCAGTAAAAGGGAGCACAAATATGAAGAGGTTGAAAGT
TAATTAAAATAAACATTAGAATTTTTTTCTATGTATATCACTTGA
TTTTTCTTTACCATAATCAAGTGATCAAATTTTAGATCTTAAATA
ATGCAATTTTAACTTGAAAGATACCCTGAGGATCCCTTATCTTTT
GATTTTTATCCCAAAGTGCAAAGTGACTTTCCAAAGGTCAGACCC
ATAACAACAGGCCAGAGCTCTACTACATTTTGTCTTTATTAGAAG
GTTTTGGCTATTTTTTTTTTCTATCAGCAATTTGTACCTCAAAAG
ACCTCATACCATACCTGTAATATTTAAGAAGATAAATTTTTTGCC
GCACTCTTAGTTTTTTTGCCCGACTCCAGTTGCAGGTAGCTACTC
TGGAAAGTCTATATAAAATACTCCTTTTCTTCTTAGCCAAATACT
TCTCCTTTTGGATCACAAATGCATTGATAATTTTCGTCTTAGTCC
CCTTAATGGTAGTAGGTGTGCCTCTCTCCCATGAACGGATATCGC
TTTATCAGTGTTAAAGTCTAAATGTTAACAGAAAAAATGAGGAAG
ATTTTATTGCCACTACTGTGAGGTTTGGGTTACCTACCTAACAAG
TGTAGCTGAACTTCCTTAGTATCATAGTAAGTGTGAGAGGAAAAT
TATTTCTGATTTTAATTTGGAAAGTTGTGTGAGTTGTAAAAAAAA
AAAAAAAAAAAATTTGCTCATTTTTCTGTGTGTCATTTGGATGAC
AAAAAATATGCTTATAAGCTTTCCTCTTTTGTTTTTATAGGAGGA
AAGAATGGAAATGATTTCTGAAAGGCCAAAAGAGAGTATGTATTC
CTGGAACAAAACTGCAGAGAAAAGTGATTTTGAAGCTGTAGAAGC
ACTTATGTCAATGAGCTGCAGTTGGAAGTCTGATTTTAAGAAATA
CGTTGAAAACAGACCTGTTACACCAGTATCTGATTTGTCAGAGGA
AGAGAATCTGCTTCCGGGAACACCTGATTTTCATACAATCCCAGC
ATTTGTAAGTATTATTGTTTTTAAGATAGACGTAAATTATATAAT
TTCTTAAAATTTATATAATAAACTGTAGATTTCTCATGCTATGTA
TTTCCCTTTTCTTTAGTGTTTGACTCCACCTTACAGTCCTTCTGA
CTTTGAACCCTCTCAAGTGTCAAATCTGATGGCACCAGCGCCATC
TACTGTACACTTCAAGTCACTCTCAGATACTGCCAAACCTCACAT
TGCCGCACCTTTCAAAGAGGAAGAAAAGAGCCCAGTATCTGCCCC
CAAACTCCCCAAAGCTCAGGCAACAAGTGTGATTCGTCATACAGC
TGATGCCCAGCTATGTAACCACCAGACCTGCCCAATGAAAGCAGC
CAGCATCCTCAACTATCAGAACAATTCTTTTAGAAGAAGAACCCA
CCTAAATGTTGAGGCTGCAAGAAAGAACATACCATGTGCCGCTGT
GTCACCAAACAGATCCAAATGTGAGAGAAACACAGTGGCAGATGT
TGATGAGAAAGCAAGTGCTGCACTTTATGACTTTTCTGTGCCTTC
CTCAGAGACGGTCATCTGCAGGTCTCAGCCAGCCCCTGTGTCCCC
ACAACAGAAGTCAGTGTTGGTCTCTCCACCTGCAGTATCTGCAGG
GGGAGTGCCACCTATGCCGGTCATCTGCCAGATGGTTCCCCTTCC
TGCCAACAACCCTGTTGTGACAACAGTCGTTCCCAGCACTCCTCC
CAGCCAGCCACCAGCCGTTTGCCCCCCTGTTGTGTTCATGGGCAC
ACAAGTCCCCAAAGGCGCTGTCATGTTTGTGGTACCCCAGCCCGT
TGTGCAGAGTTCAAAGCCTCCGGTGGTGAGCCCGAATGGCACCAG
ACTCTCTCCCATTGCCCCTGCTCCTGGGTTTTCCCCTTCAGCAGC
AAAAGTCACTCCTCAGATTGATTCATCAAGGATAAGGAGTCACAT
CTGTAGCCACCCAGGATGTGGCAAGACATACTTTAAAAGTTCCCA
TCTGAAGGCCCACACGAGGACGCACACAGGTACCAGCCACTTCTT
ATATCTTTAGTGTTTAAATGAAGAGATTTTGGCTGTGATCACACA
TTTAAACCCAGGATGATAAGGAATAACTTGCCTTTAGCTAGTTTA
GAGGATGGTATTTACCAGTGCAAGGTCCTCCAAAAGGCTGTATTG
GAGGCCGCTCACGAGTGCCATAATCCTTGCACTTTAGGAGACCAA
GGTGGGAGGATTGTTTGAGCATAGGAGTTCAAAACCAGCCTGTGC
AACATACTGAGACCTCATCTCTACAAATAATTTAAAAATAAGTAT
GTAAATACTTTATTGATGTGCTTTTGTTTCAGTCTCTCAAATGAT
GATTCTGTAGATGTCTGAGTAGTTACTTTAATAAGGTATCTTTAA
AGCCTTTGAATCATAGTTAAAGTCAGGAAGTCTTTTCATTTTTGG
CTTTTTTTAAGGGATGAAAAAAAACTGCTCTGTTGCCCAAACTGG
TCTCAAACTTCTGGGCTCAAGCTATCCTCCTGCCCCAGCCTTCTG
AGTAGTTGGGACTACAGGTGCCTACTACTCCACCCGGCTGCCTTA
TTTCATGTGGGGAAGAGTTTTGTAAATGTGATCAAGTGACAAGTT
GAAATTGGAGCAATACATATCTCATATTCATTTGACAGTTTGATT
TATTAGCTTTTATTATTTAAAATGTATQTTCTCATATACTTTTAG
ACAGAATTGAACATATAATTTACGGCAATCTTATGCATAATAATG
ATTTGTATGTCTTTGTTTTAGGAGAAAAGCCTTTCAGCTGTAGCT
GGAAAGGTTGTGAAAGGAGGTTTGCCCGTTCTGATGAACTGTCCA
GACACAGGCGAACCCACACGGGTGAGAAGAAATTTGCGTGCCCCA
TGTGTGACCGGCGGTTCATGAGGAGTGACCATTTGACCAAGCATG
CCCGGCGCCATCTATCAGCCAAGAAGCTACCAAACTGGCAGATGG
AAGTGAGCAAGCTAAATGACATTGCTCTACCTCCAACCCCTGCTC
CCACACAGTGACAGACCGGAAAGTGAAGAGTCAGAACTAACTTTG
GTCTCAGCGGGAGCCAGTGGTGATGTAAAAATGCTTCCACTGCAA
GTCTGTGGCCCCACAACGTGGGCTTAAAGCAGAAGCCCCACAGCC
TGGCACGAAGGCCCCGCCTGGGTTAGGTGACTAAAAGGGCTTCGG
CCACAGGCAGGTCACAGAAAGGCAGGTTTCATTTCTTATCACATA
AGAGAGATGAGAAAGCTTTTATTCCTTTGAATATTTTTTGAAGGT
TTCAGATGAGGTCAACACAGGTAGCACAGATTTTGAATCTGTGTG
CATATTTGTTACTTTACTTTTGCTGTTTATACTTGAGACCAACTT
TTCAATGTGATTCTTCTAAAGCACTGGTTTCAAGAATATGGAGGC
TGGAAGGAAATAAACATTACGGTACAGACATGGAGATGTAAAATG
AGTTTGTATTATTACAAATATTGTCATCTTTTTCTAGAGTTATCT
TCTTTATTATTCCTAGTCTTTCCAGTCAACATCGTGGATGTAGTG
ATTAAATATATCTAGAACTATCATTTTTACACTATTGTGAATATT
TGGAATTGAACGACTGTATATTGCTAAGAGGGCCCAAAGAATTGG
AATCCTCCTTAATTTAATTGCTTTGAAGCATAGCTACAATTTGTT
TTTGCATTTTTGTTTTGAAAGTTTAACAAATGACTGTATCTAGGC
ATTTCATTATGCTTTGAACTTTAGTTTGCCTGCAGTTTCTTGTGT
AGATTTGAAAATTGTATACCAATGTGTTTTCTGTAGACTCTAAGA
TACACTGCACTTTGTTTAGAAAAAAAACTGAAGATGAAATATATA
TTGTAAAGAAGGGATATTAAGAATCTTAGATAACTTCTTGAAAAA
GATGGCTTATGTCATCAGTAAAGTACCTTTATGTTATGAGGATAT
AATGTGTGCTTTATTGAATTAGAAAATTAGTGACCATTATTCACA
GGTGGACAAATGTTGATGTTGTCCTGTTAATTTATAGGCGTTTTT
TGGGGATGTGGAGGTAGTTGGGTAGAAAAATTATTAGAACATTCA
CTTTTGTTAACAGTATTTCTCTTTTATTCTGTTATATAGTGGATG
ATATACACAGTGGCAAAACAAAAGTACATTGCTTAAAATATATAG
TGAAAAATGTCACTATATCTTCCCATTTAACATTGTTTTTGTATA
TTGGGTGTAGATTTCTGACATCAAAACTTGGACCCTTGGAAAACA
AAAGTTTTAATTAAAAAAAATCCTTGTGACTTACAATTTGCACAA
TATTTCTTTTGTTGTACTTTATATCTTGTTTACAATAAAGAATTC
CCTTTGGTA

In some examples, the KLF10 nucleic acid includes the nucleic acid sequence of SEQ ID NO:44.

In other examples, the KLF10 nucleic acid has a nucleic acid sequence having at least 85% sequence identity (e.g., at least 85%, 86%, 87%, 88%, 89%, 90%, 91%, 92%, 93%, 94%, 95%, 96%, 97%, 98%, 99%, or 99.9% identity, or more) to the nucleic acid sequence of SEQ ID NO:44.

In some embodiments, a KLF10 cDNA has the nucleic acid sequence of SEQ ID NO:45. SEQ ID NO:45 is shown below:

AGACGGCGCTGAGCGCGGGGGGGGGGGGAGCGGCGTCGAGTGTCT
CCGTGCGCCCGTCTGTGGCCAAGCAGCCAGCAGCCTAGCAGCCAG
TCAGCTTGCCGCCGGCGGCCAAGCAGCCAACCATGCTCAACTTCG
GTGCCTCTCTCCAGCAGACTGCGGAGGAAAGAATGGAAATGATTT
CTGAAAGGCCAAAAGAGAGTATGTATTCCTGGAACAAAACTGCAG
AGAAAAGTGATTTTGAAGCTGTAGAAGCACTTATGTCAATGAGCT
GCAGTTGGAAGTCTGATTTTAAGAAATACGTTGAAAACAGACCTG
TTACACCAGTATCTGATTTGTCAGAGGAAGAGAATCTGCTTCCGG
GAACACCTGATTTTCATACAATCCCAGCATTTTGTTTGACTCCAC
CTTACAGTCCTTCTGACTTTGAACCCTCTCAAGTGTCAAATCTGA
TGGCACCAGCGCCATCTACTGTACACTTCAAGTCACTCTCAGATA
CTGCCAAACCTCACATTGCCGCACCTTTCAAAGAGGAAGAAAAGA
GCCCAGTATCTGCCCCCAAACTCCCCAAAGCTCAGGCAACAAGTG
TGATTCGTCATACAGCTGATGCCCAGCTATGTAACCACCAGACCT
GCCCAATGAAAGCAGCCAGCATCCTCAACTATCAGAACAATTCTT
TTAGAAGAAGAACCCACCTAAATGTTGAGGCTGCAAGAAAGAACA
TACCATGTGCCGCTGTGTCACCAAACAGATCCAAATGTGAGAGAA
ACACAGTGGCAGATGTTGATGAGAAAGCAAGTGCTGCACTTTATG
ACTTTTCTGTGCCTTCCTCAGAGACGGTCATCTGCAGGTCTCAGC
CAGCCCCTGTGTCCCCACAACAGAAGTCAGTGTTGGTCTCTCCAC
CTGCAGTATCTGCAGGGGGAGTGCCACCTATGCCGGTCATCTGCC
AGATGGTTCCCCTTCCTGCCAACAACCCTGTTGTGACAACAGTCG
TTCCCAGCACTCCTCCCAGCCAGCCACCAGCCGTTTGCCCCCCTG
TTGTGTTCATGGGCACACAAGTCCCCAAAGGCGCTGTCATGTTTG
TGGTACCCCAGCCCGTTGTGCAGAGTTCAAAGCCTCCGGTGGTGA
GCCCGAATGGCACCAGACTCTCTCCCATTGCCCCTGCTCCTGGGT
TTTCCCCTTCAGCAGCAAAAGTCACTCCTCAGATTGATTCATCAA
GGATAAGGAGTCACATCTGTAGCCACCCAGGATGTGGCAAGACAT
ACTTTAAAAGTTCCCATCTGAAGGCCCACACGAGGACGCACACAG
GAGAAAAGCCTTTCAGCTGTAGCTGGAAAGGTTGTGAAAGGAGGT
TTGCCCGTTCTGATGAACTGTCCAGACACAGGCGAACCCACACGG
GTGAGAAGAAATTTGCGTGCCCCATGTGTGACCGGCGGTTCATGA
GGAGTGACCATTTGACCAAGCATGCCCGGCGCCATCTATCAGCCA
AGAAGCTACCAAACTGGCAGATGGAAGTGAGCAAGCTAAATGACA
TTGCTCTACCTCCAACCCCTGCTCCCACACAGTGACAGACCGGAA
AGTGAAGAGTCAGAACTAACTTTGGTCTCAGCGGGAGCCAGTGGT
GATGTAAAAATGCTTCCACTGCAAGTCTGTGGCCCCACAACGTGG
GCTTAAAGCAGAAGCCCCACAGCCTGGCACGAAGGCCCCGCCTGG
GTTAGGTGACTAAAAGGGCTTCGGCCACAGGCAGGTCACAGAAAG
GCAGGTTTCATTTCTTATCACATAAGAGAGATGAGAAAGCTTTTA
TTCCTTTGAATATTTTTTGAAGGTTTCAGATGAGGTCAACACAGG
TAGCACAGATTTTGAATCTGTGTGCATATTTGTTACTTTACTTTT
GCTGTTTATACTTGAGACCAACTTTTCAATGTGATTCTTCTAAAG
CACTGGTTTCAAGAATATGGAGGCTGGAAGGAAATAAACATTACG
GTACAGACATGGAGATGTAAAATGAGTTTGTATTATTACAAATAT
TGTCATCTTTTTCTAGAGTTATCTTCTTTATTATTCCTAGTCTTT
CCAGTCAACATCGTGGATGTAGTGATTAAATATATCTAGAACTAT
CATTTTTACACTATTGTGAATATTTGGAATTGAACGACTGTATAT
TGCTAAGAGGGCCCAAAGAATTGGAATCCTCCTTAATTTAATTGC
TTTGAAACATAGCTACAATTTGTTTTTGCATTTTTGTTTTGAAAG
TTTAACAAATGACTGTATCTAGGCATTTCATTATGCTTTGAACTT
TAGTTTGCCTGCAGTTTCTTGTGTAGATTTGAAAATTGTATACCA
ATGTGTTTTCTGTAGACTCTAAGATACACTGCACTTTGTTTAGAA
AAAAAACTGAAGATGAAATATATATTGTAAAGAAGGGATATTAAG
AATCTTAGATAACTTCTTGAAAAAGATGGCTTATGTCATCAGTAA
AGTACCTTTATGTTATGAGGATATAATGTGTGCTTTATTGAATTA
GAAAATTAGTGACCATTATTCACAGGTGGACAAATGTTGATGTTG
TCCTGTTAATTTATAGGCGTTTTTTGGGGATGTGGAGGTAGTTGG
GTAGAAAAATTATTAGAACATTCACTTTTGTTAACAGTATTTCTC
TTTTATTCTGTTATATAGTGGATGATATACACAGTGGCAAAACAA
AAGTACATTGCTTAAAATATATAGTGAAAAATGTCACTATATCTT
CCCATTTAACATTGTTTTTGTATATTGGGTGTAGATTTCTGACAT
CAAAACTTGGACCCTTGGAAAACAAAAGTTTTAATTAAAAAAAAT
CCTTGTGACTTACAATTTGCACAATATTTCTTTTGTTGTACTTTA
TATCTTGTTTACAATAAAGAATTCCCTTTGGTAAAAAAAAAAAAA
AAAAAAAACAAAAAAAAAAAAAAAA

In some examples, the KLF10 nucleic acid includes the nucleic acid sequence of SEQ ID NO:45.

In other examples, the KLF10 nucleic acid has a nucleic acid sequence having at least 85% sequence identity (e.g., at least 85%, 86%, 87%, 88%, 89%, 90%, 91%, 92%, 93%, 94%, 95%, 96%, 97%, 98%, 99%, or 99.9% identity, or more) to the nucleic acid sequence of SEQ ID NO:45.

Any suitable vector may be used to express a KLF10 nucleic acid.

For example, in some examples, the vector is a virus. Any suitable virus may be used. Viral genomes provide a rich source of vectors that can be used for the efficient delivery of exogenous genes into a mammalian cell. Viral genomes are particularly useful vectors for gene delivery as the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors are a retrovirus (e.g., Retroviridae family viral vector), adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g., measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses are: avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology, Third Edition (Lippincott-Raven, Philadelphia, (1996)). Other examples are murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in McVey et al., (U.S. Pat. No. 5,801,030).

The vector may be a retroviral vector. One type of retroviral vector that may be used in the methods and compositions described herein is a lentiviral vector. Lentiviral vectors (LVs), a subset of retroviruses, transduce a wide range of dividing and non-dividing cell types with high efficiency, conferring stable, long-term expression of the transgene. An overview of optimization strategies for packaging and transducing LVs is provided in Delenda, The Journal of Gene Medicine 6: S125 (2004).

The use of lentivirus-based gene transfer techniques typically involves in vitro production of recombinant lentiviral particles carrying a highly deleted viral genome in which the transgene of interest is accommodated. In particular, the recombinant lentivirus are recovered through the in trans coexpression in a permissive cell line of (1) the packaging constructs, i.e., a vector expressing the Gag-Pol precursors together with Rev (alternatively expressed in trans); (2) a vector expressing an envelope receptor, generally of an heterologous nature; and (3) the transfer vector, consisting in the viral complimentary DNA (cDNA) deprived of all open reading frames, but maintaining the sequences required for replication, encapsidation, and expression, in which the sequences to be expressed are inserted.

A LV used in the methods and compositions described herein may include one or more of a 5′-Long terminal repeat (LTR), HIV signal sequence, HIV Psi signal 5′-splice site (SD), delta-GAG element, Rev Responsive Element (RRE), 3′-splice site (SA), elongation factor (EF) 1-alpha promoter and 3′-self inactivating LTR (SIN-LTR). The lentiviral vector optionally includes a central polypurine tract (cPPT) and a woodchuck hepatitis virus post-transcriptional regulatory element (WPRE), as described in U.S. Pat. No. 6,136,597, the disclosure of which is incorporated herein by reference as it pertains to WPRE. The lentiviral vector may further include a pHR′ backbone.

Enhancer elements can be used to increase expression of modified DNA molecules or increase the lentiviral integration efficiency. The LV used in the methods and compositions described herein may include a nef sequence. The LV used in the methods and compositions described herein may include a cPPT sequence which enhances vector integration. The cPPT acts as a second origin of the (+)-strand DNA synthesis and introduces a partial strand overlap in the middle of its native HIV genome. The introduction of the cPPT sequence in the transfer vector backbone strongly increased the nuclear transport and the total amount of genome integrated into the DNA of target cells. The LV used in the methods and compositions described herein may include a Woodchuck Posttranscriptional Regulatory Element (WPRE). The WPRE acts at the transcriptional level, by promoting nuclear export of transcripts and/or by increasing the efficiency of polyadenylation of the nascent transcript, thus increasing the total amount of mRNA in the cells. The addition of the WPRE to LV results in a substantial improvement in the level of transgene expression from several different promoters, both in vitro and in vivo. The LV used in the methods and compositions described herein may include both a cPPT sequence and WPRE sequence. The vector may also include an IRES sequence that permits the expression of multiple polypeptides from a single promoter.

In addition to IRES sequences, other elements which permit expression of multiple polypeptides are useful. The vector used in the methods and compositions described herein may include multiple promoters that permit expression more than one polypeptide. The vector used in the methods and compositions described herein may include a protein cleavage site that allows expression of more than one polypeptide. Examples of protein cleavage sites that allow expression of more than one polypeptide are described in Klump et al., Gene Ther. 8:811 (2001), Osborn et al., Molecular Therapy 12:569 (2005), Szymczak and Vignali, Expert Opin Biol Ther. 5:627 (2005), and Szymczak et al., Nat Biotechnol. 22:589 (2004). It will be readily apparent to one skilled in the art that other elements that permit expression of multiple polypeptides identified in the future are useful and may be utilized in the vectors suitable for use with the compositions and methods described herein.

The vector used in the methods and compositions described herein may be a clinical grade vector.

In another example, the vector is an recombinant adeno-associated virus (rAAV) vector. Nucleic acids may be incorporated into rAAV vectors and/or virions in order to facilitate their introduction into a cell. rAAV vectors useful in the compositions and methods described herein include recombinant nucleic acid constructs that include (1) a heterologous sequence to be expressed and (2) viral sequences that facilitate integration and expression of the heterologous genes. The viral sequences may include those sequences of AAV that are required in cis for replication and packaging (e.g., functional inverted terminal repeat sequences (ITR)) of the DNA into a virion. Such rAAV vectors may also contain marker or reporter genes. Useful rAAV vectors have one or more of the AAV WT genes deleted in whole or in part but retain functional flanking ITR sequences. The AAV ITRs may be of any serotype suitable for a particular application. Methods for using rAAV vectors are described, for example, in Tai et al., J. Biomed. Sci. 7:279 (2000), and Monahan and Samulski, Gene Delivery 7:24 (2000), the disclosures of each of which are incorporated herein by reference as they pertain to AAV vectors for gene delivery.

The nucleic acids and vectors described herein can be incorporated into a rAAV virion in order to facilitate introduction of the nucleic acid or vector into a cell. The capsid proteins of AAV compose the exterior, non-nucleic acid portion of the virion and are encoded by the AAV cap gene. The cap gene encodes three viral coat proteins, VP1, VP2, and VP3, which are required for virion assembly. The construction of rAAV virions has been described, for example, in U.S. Pat. Nos. 5,173,414; 5,139,941; 5,863,541; 5,869,305; 6,057,152; and 6,376,237; as well as in Rabinowitz et al., J. Virol. 76:791 (2002) and Bowles et al., J. Virol. 77:423 (2003), the disclosures of each of which are incorporated herein by reference as they pertain to AAV vectors for gene delivery.

rAAV virions useful in conjunction with the compositions and methods described herein include those derived from a variety of AAV serotypes including AAV 1, 2, 3, 4, 5, 6, 7, 8, 9, 10 and rh74. For targeting cells located in or delivered to the central nervous system, AAV2, AAV9, and AAV10 may be particularly useful. Construction and use of AAV vectors and AAV proteins of different serotypes are described, for example, in Chao et al., Mol. Ther. 2:619 (2000); Davidson et al., Proc. Natl. Acad. Sci. USA 97:3428 (2000); Xiao et al., J. Virol. 72:2224 (1998); Halbert et al., J. Virol. 74:1524 (2000); Halbert et al., J. Virol. 75:6615 (2001); and Auricchio et al., Hum. Molec. Genet. 10:3075 (2001).

Also useful in conjunction with the compositions and methods described herein are pseudotyped rAAV vectors. Pseudotyped vectors include AAV vectors of a given serotype pseudotyped with a capsid gene derived from a serotype other than the given serotype (e.g., AAV1, AAV2, AAV3, AAV4, AAV5, AAV6, AAV7, AAV8, AAV9, and AAV10, among others). Techniques involving the construction and use of pseudotyped rAAV virions are known in the art and are described, for example, in Duan et al., J. Virol. 75:7662 (2001); Halbert et al., J. Virol. 74:1524 (2000); Zolotukhin et al., Methods, 28:158 (2002); and Auricchio et al., Hum. Molec. Genet. 10:3075 (2001).

AAV virions that have mutations within the virion capsid may be used to infect particular cell types more effectively than non-mutated capsid virions. For example, suitable AAV mutants may have ligand insertion mutations for the facilitation of targeting AAV to specific cell types. The construction and characterization of AAV capsid mutants including insertion mutants, alanine screening mutants, and epitope tag mutants is described in Wu et al., J. Virol. 74:8635 (2000). Other rAAV virions that can be used in methods described herein include those capsid hybrids that are generated by molecular breeding of viruses as well as by exon shuffling. See, e.g., Soong et al., Nat. Genet., 25:436 (2000) and Kolman and Stemmer, Nat. Biotechnol. 19:423 (2001).

In some embodiments, the KLF10 agonist comprises recombinant KLF10. The recombinant KLF10 can include a wild-type KLF10 protein or a variant KLF10 protein. In some embodiments, the KLF10 protein has the amino acid sequence of SEQ ID NO:46. SEQ ID NO:46 is shown below:

MEERMEMISERPKESMYSWNKTAEKSDFEAVEALMSMSCSWKSDF
KKYVENRPVTPVSDLSEEENLLPGTPDFHTIPAFCLTPPYSPSDF
EPSQVSNLMAPAPSTVHFKSLSDTAKPHIAAPFKEEEKSPVSAPK
LPKAQATSVIRHTADAQLCNHQTCPMKAASILNYQNNSFRRRTHL
NVEAARKNIPCAAVSPNRSKCERNTVADVDEKASAALYDFSVPSS
ETVICRSQPAPVSPQQKSVLVSPPAVSAGGVPPMPVICQMVPLPA
NNPVVTTVVPSTPPSQPPAVCPPVVFMGTQVPKGAVMFVVPQPVV
QSSKPPVVSPNGTRLSPIAPAPGFSPSAAKVTPQIDSSRIRSHIC
SHPGCGKTYFKSSHLKAHTRTHTGEKPFSCSWKGCERRFARSDEL
SRHRRTHTGEKKFACPMCDRRFMRSDHLTKHARRHLSAKKLPNWQ
MEVSKLNDIALPPTPAPTQ

In other examples, the variant KLF10 protein has an amino acid sequence having at least 85% sequence identity (e.g., at least 85%, 86%, 87%, 88%, 89%, 90%, 91%, 92%, 93%, 94%, 95%, 96%, 97%, 98%, 99%, or 99.9% identity, or more) to the amino acid sequence of SEQ ID NO:46.

### D. Compositions

In another aspect, the disclosure features a pharmaceutical composition comprising a therapeutically effective amount of an anti-IL-9 antibody, or an antigen-binding fragment thereof, together with one or more pharmaceutically acceptable carriers.

In another aspect, the disclosure features a pharmaceutical composition comprising a therapeutically effective amount of a KLF10 agonist together with one or more pharmaceutically acceptable carriers.

In one embodiment, the disclosure features a composition, which is a combination of a therapeutically effective amount of an anti-IL-9 antibody or antigen-binding fragment thereof of the disclosure, or a KLF10 agonist of the disclosure, and a therapeutically effective amount of an additional therapeutic agent. The additional therapeutic agent may be an antihypertensive agent, an anti-arrhythmic agent, an anticoagulant agent, an anti-platelet agent, a cholesterol-lowering agent, digoxin, a nitrate, or any combination thereof. In some embodiments, the anti-hypertensive agent includes an angiotensin II receptor antagonist, an angiotensin-converting enzyme (ACE) inhibitor, a diuretic, a calcium channel antagonist, an adrenergic receptor antagonist, a vasodilator, a renin inhibitor, an aldosterone receptor antagonist, an alpha-2 adrenergic receptor agonist, an endothelin receptor blocker, or any combination thereof. The additional therapeutic agent may be synthetic or naturally derived.

In certain embodiments, the additional therapeutic agent may be an agent that helps to counteract or reduce any possible side effect(s) associated with an anti-IL-9 antibody or antigen-binding fragment thereof or a KLF10 agonist, if such side effect(s) should occur. It will also be appreciated that the antibodies and pharmaceutically acceptable compositions of the present disclosure can be employed in combination therapies, that is, the antibodies and pharmaceutically acceptable compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an antibody may be administered concurrently with another agent used to treat the same disorder), or they may achieve different effects (e.g., control of any adverse effects). As used herein, additional therapeutic agents that are normally administered to treat or prevent a particular disease, or condition, are appropriate for the disease, or condition, being treated.

When multiple therapeutics are co-administered, dosages may be adjusted accordingly, as is recognized in the pertinent art.

In some embodiments, the therapeutic agents can be formulated into pharmaceutical compositions for parenteral administration, e.g., formulated for injection via the subcutaneous, intravenous, intramuscular, transdermal, intravitreal, or other such routes, including peristaltic administration and direct instillation into targeted site. The preparation of an aqueous composition that contains such a therapeutic agent as an active ingredient will be known to those of skill in the art in view of this disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

The pharmaceutical forms that can be used for injectable use include sterile aqueous solutions or dispersions; formulations including sesame oil, peanut oil, or aqueous propylene glycol; and sterile powders for the extemporaneous preparation of sterile injectable solutions or dispersions. In all cases, the form should be sterile and fluid to the extent that syringability exists.

Compositions of the therapeutic agents (e.g., an anti-IL-9 antibody or a KLF10 agonist) can be formulated into a sterile aqueous composition in a neutral or salt form. Solutions as free base or pharmacologically acceptable salts can be prepared in water suitably mixed with a surfactant, such as hydroxypropylcellulose. Pharmaceutically acceptable salts, include the acid addition salts (formed with the free amino groups of the protein), and those that are formed with inorganic acids such as, for example, hydrochloric or phosphoric acids, or such organic acids as acetic, trifluoroacetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine, and the like.

Examples of carriers include solvents and dispersion media containing, for example, water, ethanol, polyol (for example, glycerol, propylene glycol, and liquid polyethylene glycol, and the like), suitable mixtures thereof, and vegetable oils. In many cases, it will be preferable to include isotonic agents, for example, sugars or sodium chloride. The proper fluidity can be maintained, for example, by using a coating, such as lecithin, by the maintenance of the required particle size in the case of dispersion and/or by using surfactants.

Formulation of the pharmaceutical compounds are known in the art and are described in Remington's Pharmaceutical Sciences (18th edition), ed. A. Gennaro, 1990, Mack Publishing Company, Easton, Pa. (also see, e.g., M. J. Rathbone, ed., Oral Mucosal Drug Delivery, Drugs and the Pharmaceutical Sciences Series, Marcel Dekker, Inc., N.Y., U.S.A., 1996; M. J. Rathbone et al., eds., Modified—Release Drug Delivery Technology, Drugs and the Pharmaceutical Sciences Series, Marcel Dekker, Inc., N.Y., U.S.A., 2003; Ghosh et al., eds., Drug Delivery to the Oral Cavity, Drugs and the Pharmaceutical Sciences Series, Marcel Dekker, Inc., N.Y., U.S.A., 2005; and Mathiowitz et al., eds., Bioadhesive Drug Delivery Systems, Drugs and the Pharmaceutical Sciences Series, Marcel Dekker, Inc., N.Y., U.S.A., 1999.

Pharmaceutical compositions for use in the methods described herein can have a therapeutically effective amount of the agent in a dosage in the range of 0.01 to 1,000 mg/kg of body weight of the subject, and more preferably in the range of from about 1 to 100 mg/kg of body weight of the patient. In certain embodiments, the pharmaceutical compositions for use in the methods of the present disclosure have a therapeutically effective amount of the agent in a dosage in the range of 1 to 10 mg/kg of body weight of the subject.

Accordingly, the present disclosure provides a composition, e.g., a pharmaceutical composition, containing the anti-IL-9 antibodies, or antibody fragments thereof, or KLF10 agonists disclosed herein. The antibodies or KLF10 agonists, if desired, may be modified according to any of the modifications outlined above. The pharmaceutical compositions may be formulated together with a pharmaceutically acceptable carrier, excipient, or diluent.

A pharmaceutical composition described herein can be administered by a variety of methods known in the art. As will be appreciated by the skilled artisan, the route and/or mode of administration will vary depending upon the desired results. The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are generally known to those skilled in the art.

To administer a compound of the disclosure (e.g., an anti-IL-9 antibody or a KLF10 agonist) by certain routes of administration, it may be necessary to coat the compound with, or co-administer the compound with, a material to prevent its inactivation. For example, the compound may be administered to a subject in an appropriate carrier, for example, liposomes, or a diluent. Pharmaceutically acceptable diluents include saline and aqueous buffer solutions. Liposomes include water-in-oil-in-water CGF emulsions as well as conventional liposomes.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the disclosure is contemplated. Supplementary active compounds can also be incorporated into the compositions.

Active ingredients of the pharmaceutical composition may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nano capsules) or in macroemulsions.

Sustained-release preparations may be prepared. Suitable examples of sustained-release preparations include semipermeable matrices of solid hydrophobic polymers containing the antibody or KLF10 agonist, which matrices are in the form of shaped articles, for example, films, or microcapsules.

The formulations to be used for in vivo administration are generally sterile. Sterility may be readily accomplished, e.g., by filtration through sterile filtration membranes. Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof. Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, such as TWEEN® 80. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Alternatively, genes encoding the anti-IL-9 antibodies or KLF10 agonists may be delivered directly into the subject for expression rather than administering purified antibodies for prevention or therapy. For example, viral vectors, such as recombinant viruses, can be used to deliver the heavy and light chain genes. In one example, rAAV virus particles can be used to deliver anti-HIV monoclonal antibodies (Balazs et al. Nature. 481: 81, 2012). Antibody genes could also be effectively delivered by electroporation of muscle cells with plasmid DNA containing heavy and/or light chain genes (e.g., VH and/or VL genes) (Muthumani et al. Hum Vaccin Immunother. 10: 2253, 2013). Lentivirus vectors or other nucleic acids (e.g., RNA) capable of delivering transgenes could also be used to delivery antibody genes to establish serum antibody levels capable of prevention.

Also contemplated are kits including anti-IL-9 antibodies and, optionally, instructions for use. Also contemplated are kits including KLF10 agonists and, optionally, instructions for use. The kits can further contain one or more additional therapeutic agents, such as an antihypertensive agent, an anti-arrhythmic agent, an anticoagulant agent, an anti-platelet agent, a cholesterol-lowering agent, digoxin, a nitrate, or any combination thereof.

### D. Therapy

Any of the anti-IL-9 antibodies or KLF10 agonists described herein and compositions containing the antibodies can be used in a variety of in vitro and in vivo therapeutic applications. In some embodiments, an anti-IL-9 antibody or a KLF10 agonist may be used as a monotherapy. In some embodiments, an anti-IL-9 antibody or a KLF10 agonist may be used as a combination therapy.

The disclosure provides an anti-IL-9 antibody for use as a medicament. In further aspects, an anti-IL-9 antibody for use in treating a hypertensive disease or condition, a cardiovascular disease, chronic kidney disease, or fibrosis (for e.g., perivascular fibrosis) is provided. In certain embodiments, an anti-IL-9 antibody for use in a method of treatment is provided. In certain embodiments, the disclosure provides an anti-IL-9 antibody for use in a method of treating an individual having a hypertensive disease or condition, a cardiovascular disease, chronic kidney disease, or fibrosis (for e.g., perivascular fibrosis) comprising administering to the individual an effective amount of the anti-IL-9 antibody. In one such embodiment, the method further comprises administering to the individual an effective amount of at least one additional therapeutic agent, for example, as described herein.

The disclosure provides a KLF10 agonist for use as a medicament. In further aspects, a KLF10 agonist for use in treating a hypertensive disease or condition, a cardiovascular disease, chronic kidney disease, or fibrosis (for e.g., perivascular fibrosis) is provided. In certain embodiments, a KLF10 agonist for use in a method of treatment is provided. In certain embodiments, the disclosure provides a KLF10 agonist for use in a method of treating an individual having a hypertensive disease or condition, a cardiovascular disease, chronic kidney disease, or fibrosis (for e.g., perivascular fibrosis) comprising administering to the individual an effective amount of the KLF10 agonist. In one such embodiment, the method further comprises administering to the individual an effective amount of at least one additional therapeutic agent, for example, as described herein.

The disclosure provides an anti-IL-9 antibody in the manufacture or preparation of a medicament. In a further aspect, the disclosure provides for the use of an anti-IL-9 antibody in the manufacture or preparation of a medicament. In one embodiment, the medicament is for treatment of a hypertensive disease or condition, a cardiovascular disease, chronic kidney disease, or fibrosis (for e.g., perivascular fibrosis). In a further embodiment, the medicament is for use in a method of treating a hypertensive disease or condition, a cardiovascular disease, chronic kidney disease, or fibrosis (for e.g., perivascular fibrosis), e.g., comprising administering to an individual having a hypertensive disease or condition, a cardiovascular disease, chronic kidney disease, or fibrosis an effective amount of the medicament. In one such embodiment, the method further comprises administering to the individual an effective amount of at least one additional therapeutic agent, e.g., as described herein.

The disclosure provides a KLF10 agonist in the manufacture or preparation of a medicament. In a further aspect, the disclosure provides for the use of a KLF10 agonist in the manufacture or preparation of a medicament. In one embodiment, the medicament is for treatment of a hypertensive disease or condition, a cardiovascular disease, chronic kidney disease, or fibrosis (for e.g., perivascular fibrosis). In a further embodiment, the medicament is for use in a method of treating a hypertensive disease or condition, a cardiovascular disease, chronic kidney disease, or fibrosis (for e.g., perivascular fibrosis), e.g., comprising administering to an individual having a hypertensive disease or condition, a cardiovascular disease, chronic kidney disease, or fibrosis an effective amount of the medicament. In one such embodiment, the method further comprises administering to the individual an effective amount of at least one additional therapeutic agent, e.g., as described herein.

In a further aspect, the disclosure provides a method for treating a hypertensive disease or condition, a cardiovascular disease, chronic kidney disease, or fibrosis (for e.g., perivascular fibrosis). In some instances, the method comprises administering the individual having such a hypertensive disease or condition, a cardiovascular disease, chronic kidney disease, or fibrosis (for e.g., perivascular fibrosis) an effective amount of an anti-IL-9 antibody. In one embodiment, the method comprises administering to an individual having such a hypertensive disease or condition, a cardiovascular disease, chronic kidney disease, or fibrosis (e.g., perivascular fibrosis) an effective amount of an anti-IL-9 antibody (e.g., any anti-IL-9 antibody disclosed herein). In one such embodiment, the method further comprises administering to the individual an effective amount of at least one additional therapeutic agent, as described herein. In some embodiments, the additional therapeutic agent includes an antihypertensive agent, an anti-arrhythmic agent, an anticoagulant agent, an anti-platelet agent, a cholesterol-lowering agent, digoxin, a nitrate, or any combination thereof.

In a further aspect, the disclosure provides a method for treating a hypertensive disease or condition, a cardiovascular disease, chronic kidney disease, or fibrosis (for e.g., perivascular fibrosis). In some instances, the method comprises administering the individual having such a hypertensive disease or condition, a cardiovascular disease, chronic kidney disease, or fibrosis (for e.g., perivascular fibrosis) an effective amount of a KLF10 agonist. In one embodiment, the method comprises administering to an individual having such a hypertensive disease or condition, a cardiovascular disease, chronic kidney disease, or fibrosis (e.g., perivascular fibrosis) an effective amount of a KLF10 agonist (e.g., any KLF10 agonist disclosed herein). In one such embodiment, the method further comprises administering to the individual an effective amount of at least one additional therapeutic agent, as described herein. In some embodiments, the additional therapeutic agent includes an antihypertensive agent, an anti-arrhythmic agent, an anticoagulant agent, an anti-platelet agent, a cholesterol-lowering agent, digoxin, a nitrate, or any combination thereof.

In a further aspect, the disclosure provides pharmaceutical formulations comprising any of the anti-IL-9 antibodies provided herein, e.g., for use in any of the above therapeutic methods. In one embodiment, a pharmaceutical formulation comprises any of the anti-IL-9 antibodies provided herein and a pharmaceutically acceptable carrier.

In a further aspect, the disclosure provides pharmaceutical formulations comprising any of the KLF10 agonists provided herein, e.g., for use in any of the above therapeutic methods. In one embodiment, a pharmaceutical formulation comprises any of the KLF10 agonists provided herein and a pharmaceutically acceptable carrier.

In another aspect, the disclosure features an anti-IL-9 antibody, or an antigen-binding fragment thereof, for use in treating a hypertensive disease or condition, a cardiovascular disease, or chronic kidney disease in a subject in need thereof, and/or reducing fibrosis (e.g., perivascular fibrosis) in a subject in need thereof.

In another aspect, the disclosure features a KLF10 agonist, for use in treating a hypertensive disease or condition, a cardiovascular disease, or chronic kidney disease in a subject in need thereof, and/or reducing fibrosis (e.g., perivascular fibrosis) in a subject in need thereof.

In a further aspect, the disclosure features a method of treating a subject having a hypertensive disease or condition, a cardiovascular disease, chronic kidney disease, or fibrosis (for e.g., perivascular fibrosis) comprising administering a therapeutically effective amount of an antibody (e.g., a human monoclonal antibody) that specifically binds to IL-9 or a pharmaceutical composition thereof, thereby treating the subject.

In a further aspect, the disclosure features a method of treating a subject having a hypertensive disease or condition, a cardiovascular disease, chronic kidney disease, or fibrosis (for e.g., perivascular fibrosis) comprising administering a therapeutically effective amount of a KLF10 agonist or a pharmaceutical composition thereof, thereby treating the subject.

In some embodiments, the hypertensive disease or condition is a heart disease or a kidney disease. In some embodiments, the hypertensive disease or condition includes hypertension; hypertensive heart disease; heart failure with preserved ejection fraction; coronary heart disease; hypertensive-associated end organ damage; or any combination thereof; the cardiovascular disease includes coronary artery disease, atherosclerosis, myocardial infarction, heart failure, atrial fibrillation, cerebrovascular disease, stroke, peripheral artery disease, aortic aneurysm, retinopathy, or any combination thereof; or the chronic kidney disease includes ESRD. In some embodiments, the hypertension includes isolated systolic, malignant, or resistant hypertension.

In some examples, the method of treatment further comprises administering an effective amount of an additional therapeutic agent useful for ameliorating the symptoms of a hypertensive disease or condition, a cardiovascular disease, chronic kidney disease, or reducing fibrosis (for e.g., perivascular fibrosis). In some instances, the additional therapeutic agent is selected from the group consisting of an antihypertensive agent, an anti-arrhythmic agent, an anticoagulant agent, an anti-platelet agent, a cholesterol-lowering agent, digoxin, a nitrate, or any combination thereof. In some embodiments, the anti-hypertensive agent includes an angiotensin II receptor antagonist (e.g., Azilsartan, Candesartan, Eprosartan, Irbesartan, Losartan, Olmesartan, Telmisartan, Valsartan), an angiotensin-converting enzyme (ACE) inhibitor (e.g., Benazepril, Captopril, Enalapril, Fosinopril, Lisinopril, Moexipril, Perindopril, Quinapril, Ramipril, Trandolapril), a diuretic (e.g., Chlorothiazide, Chlorthalidone, Hydrochlorothiazide, Indapamide, Metolazone, Bumetanide, Ethacrynic acid, Furosemide, Torsemide, Amiloride, Eplerenone, Spironolactone, Triamterene), a calcium channel antagonist (e.g., Amlodipine, Diltiazem, Felodipine, Isradipine, Nicardipine, Nifedipine, Nisoldipine, Verapamil), an adrenergic receptor antagonist (e.g., Terazosin, doxazosin, alfuzosin, tamsulosin, silodosin, esmolol, betaxolol, metoprolol, dapiprazole, atenolol, mirtazapine, timolol, profenamine, prazosin), a vasodilator (e.g., ACE inhibitors, angiotensin receptor blockers (ARBs), calcium channel blockers (CCBs), nitrates), a renin inhibitor (e.g., Enalkiren, VTP-27999, Remikiren, Aliskiren), an aldosterone receptor antagonist (e.g., Spironolactone, Eplerenone, Finerenone), an alpha-2 adrenergic receptor agonist (e.g., clonidine, tizanidine, dexmedetomidine), an endothelin receptor blocker (e.g., ambrisentan, bosentan, macitentan), or any combination thereof.

Such combination therapies encompass combined administration (where two or more therapeutic agents are included in the same or separate formulations), and separate administration, in which case, administration of the antibody or KLF10 agonist of the disclosure can occur prior to, simultaneously, and/or following, administration of the additional therapeutic agent or agents. In one embodiment, administration of the anti-IL-9 antibody or KLF10 agonist and administration of an additional therapeutic agent occur within about one month, or within about one, two or three weeks, or within about one, two, three, four, five, or six days, of each other.

Anti-IL-9 antibodies described herein or KLF10 agonists described herein may also be used in combination.

Typically, treatment results in an improvement in heart function, kidney function, or vascular remodeling compared to a subject who has not been treated with the anti-IL-9 antibody or the antigen-binding fragment thereof or the KLF10 agonist. The treatment can also result in a decreased fibroblast intracellular calcium mobilization, a decreased fibroblast activation or differentiation, a reduced production of one or more extracellular matrix (ECM) components, an improved left ventricular global longitudinal strain (LV GLS), a decreased pulse wave velocity (PWV), an increased circumferential (Circ) strain, a decreased ratio of albumin to creatinine, a decreased kidney injury molecule-1 (KIM-1) expression level, a decreased calcium deposition in the perivascular adventitia or any combination of the above improvements, compared to a subject who has not been treated with the anti-IL-9 antibody or the antigen-binding fragment thereof or the KLF10 agonist.

In some embodiments, a therapeutic amount of the therapeutic agent can be administered to a subject to neutralize IL-9 expression and treat a disease or disorder associated with a hypertensive condition or disorder. In some embodiments, the disease or disorder is a heart or kidney disease. In some embodiments, the heart or kidney disease is perivascular fibrosis; hypertensive heart disease; isolated systolic, malignant, or resistant hypertension; heart failure with preserved ejection fraction; chronic kidney disease; or coronary heart disease. A therapeutic amount is an amount capable of producing a medically desirable result in a treated animal or human. As is well known in the art the subject's size, body surface area, age, the particular composition to be administered, sex, time and route of administration, general health, and other drugs being administered concurrently. Specific dosages of proteins and nucleic acids can be determined readily determined by one skilled in the art using the experimental methods described below.

An antibody, e.g., as described herein, or a KLF10 agonist, e.g., as described herein, can be administered by any suitable means, including parenteral, intrapulmonary, intranasal, oral, mucosal, intravenous, and, if desired for local treatment, intralesional administration. Parenteral infusions include intramuscular, intravenous, intraarterial, intraperitoneal, or subcutaneous administration. In some instances, an anti-IL-9 antibody (e.g., any anti-IL-9 antibody disclosed herein) or a KLF10 agonist may be administered orally, intrarectally, mucosally, intravenously, intramuscularly, intradermally, transdermally, subcutaneously, percutaneously, intraarterially, intraperitoneally, intravitreally, topically, intralesionally, intraarticularly, intraprostatically, intrapleurally, intratracheally, intrathecally, intranasally, intravaginally, intratumorally, intraperitoneally, peritoneally, intraventricularly, intracranially, subconjunctivally, intravesicularly, intrapericardially, intraumbilically, intraorbitally, ocularly, intraocularly, juxtasclerally, subtenonly, superchoroidally, by inhalation, by injection, by eye drop, by implantation, by infusion, by continuous infusion, by localized perfusion bathing target cells directly, by catheter, by lavage, in cremes, or in lipid compositions. In certain instances, antibody genes (e.g., genes encoding any one or more of the anti-IL-9 antibodies of the disclosure could be administered as a gene therapy to produce the one or more anti-IL-9 antibodies in the subject using either DNA vectors or viral vectors (e.g., rAAV vectors). Dosing can be by any suitable route, for example, by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies of the disclosure or KLF10 agonists of the disclosure would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody or KLF10 agonist need not be but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of a hypertensive disease or condition, a cardiovascular disease, or chronic kidney disease, or a subject who has fibrosis (for e.g., perivascular fibrosis), the appropriate dosage of an antibody of the disclosure or a KLF10 agonist of the disclosure (when used alone or in combination with one or more other additional therapeutic agents) will depend on the severity of the symptoms to be prevented/treated, the duration of effective antibody concentration required, the type of antibody, the severity and course of the disease, whether the antibody or KLF10 agonist is administered for preventive or therapeutic purposes, previous therapy, the patient's clinical history and response to the antibody, and the discretion of the attending physician. The antibody or KLF10 agonist is suitably administered to the patient at one time or over a series of treatments. For repeated administrations over several days or longer, depending on the condition, the treatment would generally be sustained until a desired suppression of disease symptoms occurs. Doses may be administered intermittently, e.g., every week, every two weeks, every three weeks, every four weeks, every five weeks, every six weeks, every seven weeks, every eight weeks, every nine weeks, every ten weeks, every eleven weeks, or every twelve weeks (e.g., such that the patient receives from about two to about twenty, or e.g., about six doses of the antibody). For example, a dose may be administered once per month, once every two months, or once every three months (e.g., by intravenous or subcutaneous injection) as an initial higher loading dose, followed by one or more lower doses may be administered. However, other dosage regimens may be useful. The progress of this therapy is easily monitored by conventional techniques and assays.

The overall dosage will be a therapeutically effective amount depending on several factors including the particular agent used, overall health of a subject, the subject's disease state, severity of the condition, the observation of improvements, and the formulation and route of administration of the selected agent(s). Determination of a therapeutically effective amount is within the capability of those skilled in the art. The exact formulation, route of administration and dosage can be chosen by the individual physician in view of the subject's condition.

Actual dosage levels of the active ingredients in the pharmaceutical compositions of the present disclosure may be varied to obtain an amount of the active ingredient which is effective to achieve the desired therapeutic response and duration for a particular patient, composition, and mode of administration, without being toxic to the patient. The selected dosage level will depend upon a variety of pharmacokinetic factors including the activity of the particular compositions of the present disclosure employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. A physician having ordinary skill in the art can readily determine and prescribe the effective amount of the pharmaceutical composition required. For example, the physician can start doses of the compounds of the disclosure employed in the pharmaceutical composition at levels lower than that required in order to achieve the desired therapeutic effect and gradually increase the dosage until the desired effect is achieved. In general, a suitable daily dose of compositions of the disclosure will be that amount of the compound which is the lowest dose effective to produce a therapeutic effect. Such an effective dose will generally depend upon the factors described above. In some examples, the administration is intravenous, intramuscular, intraperitoneal, or subcutaneous, e.g., administered proximal to the site of the target. If desired, the effective daily dose of therapeutic compositions may be administered as two, three, four, five, six or more sub-doses administered separately at appropriate intervals throughout the day, optionally, in unit dosage forms. While it is possible for a compound of the present disclosure to be administered alone, it is preferable to administer the compound as a pharmaceutical formulation (composition).

Therapeutic compositions can be administered with medical devices known in the art. For example, in a preferred embodiment, a therapeutic composition of the disclosure can be administered with a needleless hypodermic injection device, such as the devices disclosed in U.S. Pat. Nos. 5,399,163, 5,383,851, 5,312,335, 5,064,413, 4,941,880, 4,790,824, or 4,596,556. Examples of well-known implants and modules useful in the present disclosure include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known to those skilled in the art.

In some instances, the antibody-based or KLF10 agonist-based therapy may be combined with an additional therapy for more efficacious treatment (e.g., additive, or synergistic treatment) of the subject. Accordingly, subjects treated with antibodies or KLF10 agonists of the disclosure can be additionally administered (prior to, simultaneously with, or following administration of an antibody or KLF10 agonist of the disclosure) with another therapeutic agent which enhances or augments the therapeutic effect of the antibodies or KLF10 agonists.

### E. Articles of Manufacture

In another aspect, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). In some embodiments, least one active agent in the composition is an antibody or KLF10 agonist of the disclosure. The label or package insert indicates that the composition is used for treating the condition of choice. Moreover, the article of manufacture may comprise (a) a first container with a composition contained therein, wherein the composition comprises an antibody or KLF10 agonist of the disclosure; and (b) a second container with a composition contained therein, wherein the composition comprises a further cytotoxic or otherwise therapeutic agent. The article of manufacture in this embodiment of the disclosure may further comprise a package insert indicating that the compositions can be used to treat a particular condition. Alternatively, or additionally, the article of manufacture may further comprise a second (or third) container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes. In some embodiments, the article of manufacture comprises an additional therapeutic agent (e.g., a corticosteroid, a bronchial dilator, an antihistamine, epinephrine, and/or a decongestant).

Other embodiments of the present disclosure are described in the following Examples. The present disclosure is further illustrated by the following examples which should not be construed as further limiting. The contents of the appendix, and all references, patents and published patent applications cited throughout this application are expressly incorporated herein by reference.

### IIII. EXAMPLES

The following are examples of the methods of the invention. It is understood that various other embodiments may be practiced, given the general description provided above.

Table of Contents (Examples):

| | |
|---|---|
| Example 1. | KLF10 expression is increased in CD4+ T cells after Ang II treatment. |
| Example 2. | KLF10 deficiency in CD4+ T cells impairs the function of hypertension-related |
| | organs and triggers perivascular fibrosis independent of blood pressure. |
| Example 3. | KLF10 deficient CD4+ T cells release IL-9 that mediates perivascular fibrosis. |
| Example 4. | KLF10 binds to the IL-9 promoter and interacts with HDAC1 to inhibit IL-9 |
| | activation. |
| Example 5. | Transcriptomic changes involved in Ang II-induced perivascular fibrosis. |
| Example 6. | TKO fibroblasts display an activation signature and IL-9 and Ang II treatment |
| | recapitulate the phenotype in control fibroblasts. |
| Example 7. | Single-cell RNA sequencing revealed fibroblast heterogeneity and activation |
| | signatures induced in TKO aortas. |
| Example 8. | Neutralization of endogenous IL-9 reversed the Ang II-induced perivascular |
| | fibrosis and ameliorated injury of hypertension-related organs. |
| Example 9. | Materials and Methods |

### Example 1: KLF10 Expression is Increased in CD4+ T Cells after Ang II Treatment

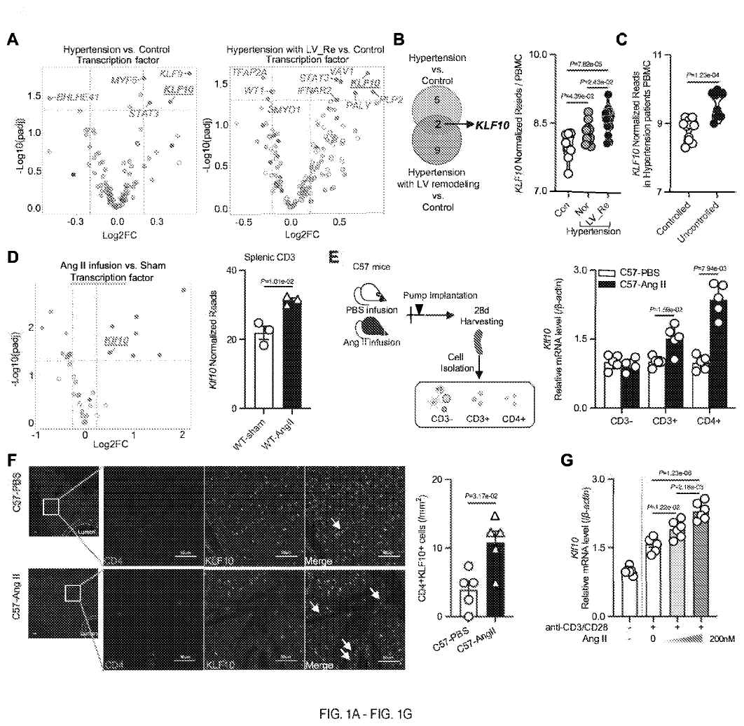

Given the importance of transcriptional control of leukocytes in hypertension, dysregulated transcription factors (TFs) were examined from a published dataset of hypertensive patients with or without left ventricular (LV) remodeling in peripheral blood mononuclear cells (PBMCs). Multiple TFs were found dysregulated in hypertensive patients with or without LV remodeling compared with control individuals (FIG. 1A). However, only two TFs (KLF10 and STAT3) were found commonly dysregulated in hypertensive patients with or without LV remodeling compared to controls (FIG. 1B). The increased expression of KLF10 was further confirmed in an independent dataset of uncontrolled hypertensive subjects (FIG. 1C). Considering the important regulatory role of KLF10 in CD4+ T cells in atherosclerosis, obesity and insulin resistance, we sought to investigate if Klf10 expression was altered in T cells in a murine model of hypertension. RNA-sequencing from Ang II-treated mice revealed Kif10 to be one of the top TFs significantly increased in splenic CD3+ T cells (FIG. 1D). This was confirmed by RT-qPCR analysis of isolated CD3− cells, CD3+, and CD4+ T cells from the spleen after 28 days of Ang II treatment in which Kif10 expression increased in response to Ang II in CD3+ T cells and even higher in CD4+ T cells (FIG. 1E). Because Ang II treatment is known to impact vascular remodeling, co-staining of KLF10 and CD4+ T cells were performed in cross-sections of the descending aorta. Higher counts of CD4+KLF10+ cells were observed after Ang II treatment compared with the PBS infused group (FIG. 1F). To identify whether Ang II can increase Kif10 in T cells, CD4+ T cells were isolated from C57BL/6 mice and treated with increasing amounts of Ang II. Kif10 expression was found to be significantly up-regulated by Ang II in a dose-dependent manner (FIG. 1G). Taken together, KLF10 expression increased in PBMCs in hypertensive patients, and in CD4+ T cells after Ang II treatment in mice, highlighting a potential role of Kif10 in CD4+ T cell in Ang II-induced vascular remodeling.

### Example 2: KLF10 Deficiency in CD4+ T Cells Impairs the Function of Hypertension-Related Organs and Triggers Perivascular Fibrosis Independent of Blood Pressure

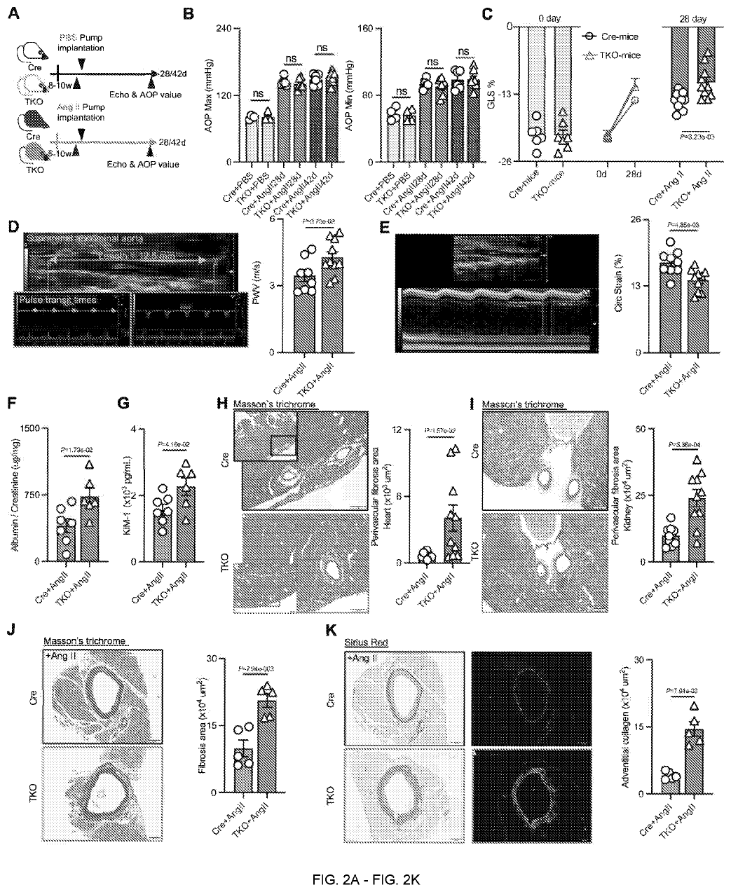

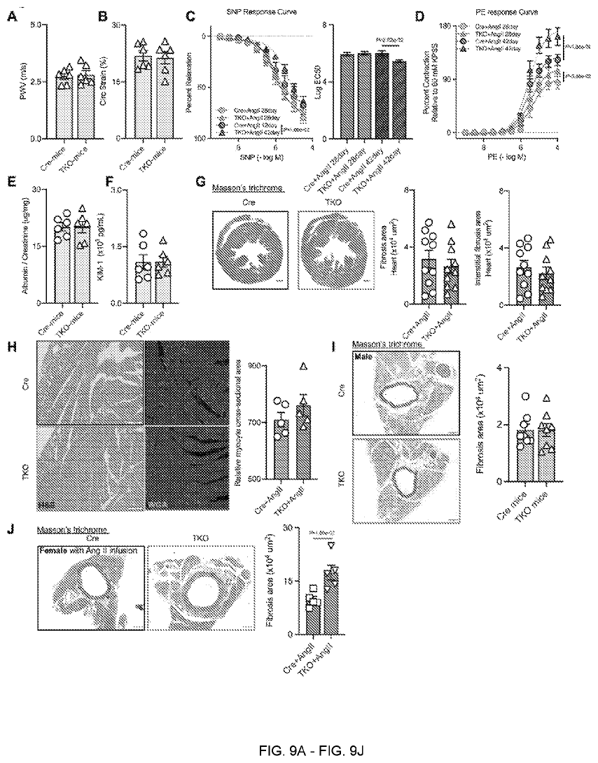

To evaluate the role of KLF10 in CD4+ T cells in Ang II-induced vascular remodeling, CD4-targeted KLF10 deficient (Klf10^{fl/fl}CD4^{Cre+}, (TKO)) and CD4-Cre (Klf10^{+/+}CD4^{Cre+}, (Cre)) control mice were infused with PBS or Ang II for 28 or 42 days (FIG. 2A). Aortic blood pressure (AOP) increased in the Ang II treatment groups compared to PBS controls; however, no difference was found between TKO and Cre control mice in both 28 days and 42 days (FIG. 2B). Considering Ang II-induced hypertension can cause tissue remodeling and multi-organ dysfunction, we further evaluated the function of heart, aorta, and kidney in these mice. After 28 days of Ang II infusion, hearts of TKO mice demonstrated impaired LV global longitudinal strain (GLS, FIG. 2C, Table 2); furthermore, aortas of TKO mice developed increased abdominal pulse-wave velocity (PWV, FIG. 2D) an decreased circumferential strain (Circ Strain, FIG. 2E) compared to Cre control mice, while there were no differences prior to Ang II infusion (FIGS. 9A and 9B). Similarly, following 28 days of Ang II infusion, TKO mice demonstrated smooth muscle hypercontractility compared to Cre control mice, and both smooth muscle hypercontractility and impaired smooth muscle relaxation manifested after 42 days of Ang II infusion (FIGS. 9C and 9D). Ang II infusion also resulted in a marked increase of albuminuria and kidney injury molecule-1 (KIM-1) levels as detected in the urine of TKO mice (FIGS. 2F and 2G), while no statistical difference was observed under basal conditions compared with control mice (FIGS. 9E and 9F). Altogether, our results suggest end organ dysfunction and likely tissue remodeling occurred after Ang II infusion in mice deficient of KI/10 in CD4+ T cells, effects that were independent of blood pressure differences between TKO and Cre control mice. Table 2 is shown below.

TABLE 2
Parameters of cardiac physiology by echocardiography in
Cre or TKO mice treated with PBS or Ang II for 28 days

| | PBS group | | Ang II group | |
|---|---|---|---|---|
| | Cre mice | TKO mice | Cre mice | TKO mice |
| Parameters | (n = 6) | (n = 6) | (n = 10) | (n = 10) |
| EF(Mean ± SD), % | 86.22 ± 2.97 | 86.23 ± 5.46 | 86.20 ± 5.04 | 88.98 ± 3.64 |
| FS(Mean ± SD), % | 54.13 ± 3.93 | 54.59 ± 6.80 | 54.55 ± 6.94 | 57.90 ± 4.97 |
| LV Mass(Mean ± SD), mg | 92.18 ± 5.63 | 95.16 ± 14.72 | 128.44 ± 6.31** | 130.28 ± 7.75*** |
| LVEDD (Mean ± SD), mm | 2.61 ± 0.23 | 2.59 ± 0.06 | 2.57 ± 0.16 | 2.59 ± 0.21 |
| LVESD (Mean ± SD), mm | 1.20 ± 0.12 | 1.19 ± 0.17 | 1.17 ± 0.22 | 1.19 ± 0.26 |
| LVAW; d (Mean ± SD), mm | 1.18 ± 0.07 | 1.15 ± 0.10 | 1.37 ± 0.09** | 1.40 ± 0.14** |
| LVAW; s (Mean ± SD), mm | 1.87 ± 0.12 | 1.80 ± 0.11 | 2.08 ± 0.16** | 2.14 ± 0.16* |
| LVPW; d (Mean ± SD), mm | 0.97 ± 0.16 | 1.01 ± 0.11 | 1.18 ± 0.09** | 1.21 ± 0.14* |
| LVPW; s (Mean ± SD), mm | 1.62 ± 0.21 | 1.67 ± 0.17 | 1.80 ± 0.07* | 1.83 ± 0.09* |
| E/A (Mean ± SD) | 1.74 ± 0.54 | 1.77 ± 0.47 | 1.24 ± 0.47* | 1.20 ± 0.10* |
| E/e′ (Mean ± SD) | 21.44 ± 4.65 | 19.61 ± 2.55 | 30.19 ± 5.68** | 31.34 ± 4.82*** |

EF, Ejection fraction; FS; fractional shortening; LV, Left ventricular; LVEDV/LVESV, LV end-diastolic/end systolic volumes; LVAW; d/s, LV end-diastolic/systolic anterior wall thickness; LVPW; d/s LV end-diastolic/systolic posterior wall thickness.

As a cardiovascular damage inducer, chronic Ang II infusion can induce significant hypertension, cardiomyocyte hypertrophy, and fibrosis in mice. To identify histopathological changes, we first evaluated the hearts and found no histopathological difference in total fibrosis, interstitial fibrosis, and myocyte cross-sectional area between Cre and TKO groups after Ang II infusion (FIGS. 9G and 9H). However, perivascular fibrosis in the heart was markedly increased in TKO mice after Ang II treatment compared to Cre mice (FIG. 2H). Similarly, increased perivascular fibrosis was also observed in the kidneys of TKO mice (FIG. 2I). Furthermore, severe aortic perivascular fibrosis and adventitial collagen deposition were detected in Ang II treated TKO mice by Masson trichrome staining and Sirius red staining (FIGS. 2J and 2K). No differences in fibrosis or collagen were observed between TKO and Cre before Ang II treatment (FIG. 9I). Analogous perivascular phenotypes were also found in female mice (FIG. 9J). Collectively, our results indicate that mice deficient of Klf10 in CD4+ T cells (TKO) develop accelerated perivascular fibrosis compared to Cre control mice in aortas, hearts, and kidneys after Ang II infusion.

### Example 3: KLF10 Deficient CD4+ T Cells Release IL-9 that Mediates Perivascular Fibrosis

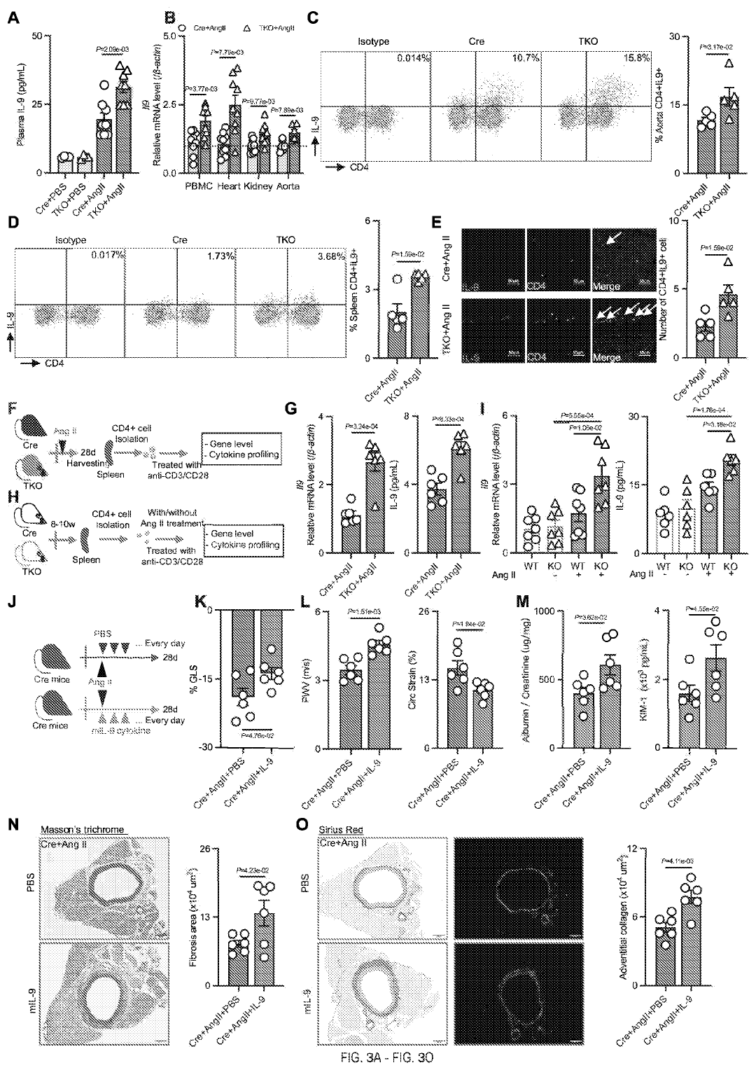

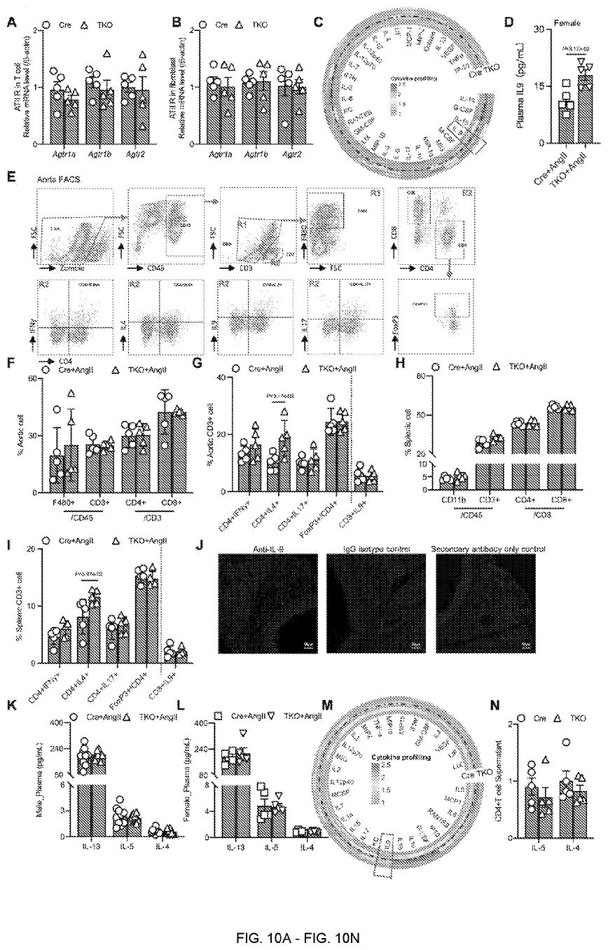

To elucidate possible mediators involved with the observed multi-organ perivascular fibrosis, we first examined the expression of angiotensin II receptors in CD4+ T cells and fibroblasts; however, no differences were observed between the TKO and Cre control groups (FIGS. 10A and 10B). To identify potential mediators in the circulation, we performed plasma cytokine profiling from Ang II treated TKO and Cre mice (FIG. 10C). Amongst a panel of inflammatory markers, IL-9 was the only significantly increased cytokine after Ang II treatment in both male and female TKO mice compared to controls (FIGS. 3A and 10D). In concordance, 119 expression was also upregulated in PBMCs, hearts, kidneys, and aortas (FIG. 3B). Considering that Ang II treatment is known to increase the accumulation of leukocytes and T cells in tissues to mediate vascular dysfunction, hypertension, and end-organ inflammation, we sought to determine the role of CD4+IL9+ cells in vascular inflammation. Flow cytometry from single-cell suspensions of the thoracic aorta showed that the percentage of CD4+IL9+ cells and CD4+IL4+ cells increased in TKO mice after Ang II treatment, while no differences were observed in other cell types compared to Cre control including CD8+IL9+ cells (FIGS. 3C and 10E through 10G). Similar changes were found in the spleen (FIGS. 3D, 10H, and 10I). Consistently, immunofluorescence imaging also captured the increase of CD4+IL9+ cells in the periadventitial tissue (FIGS. 3E and 10J). Although the percentage of CD4+IL4+ T cells increased in TKO mice, there were no differences of released IL-13, IL-5, and IL-4 in both male and female TKO mice compared with Cre controls (FIGS. 10K and 10L). To confirm whether KLF10 deficiency in CD4+ T cells affected the production of IL-9, CD4+ T cells were isolated from Ang II-treated TKO and Cre mice (FIG. 3F). CD4+ T cells from TKO mice had higher mRNA expression for 119 and released more IL-9 protein in supernatants compared to Cre controls (FIGS. 3G and 10M), while no differences were observed in secreted IL-5 and IL-4 (FIG. 10N). Similarly, CD4+ T cells isolated from TKO or Cre mice and subsequently treated with Ang II ex vivo for 1 day (FIG. 3H) demonstrated increased 119 expression and had a higher level of IL-9 protein release in supernatants from TKO CD4+ T cells compared to Cre CD4+ T cells (FIG. 3I).

To further assess the role of IL-9 in the development of perivascular fibrosis, recombinant mIL9 was administrated in Ang II-treated Cre mice (FIG. 3J). Systemic delivery of mIL9 markedly impaired LV GLS (FIG. 3K, Table 3), increased PWV, and decreased Circ Strain (FIG. 3L) in Cre mice. In addition, the ratio of albumin and creatinine, and the value of KIM-1 increased after treating with mIL9 (FIG. 3M). Importantly, histopathological analyses confirmed that the mIL9 treated group developed severe perivascular fibrosis and increased adventitial collagen deposition (FIGS. 3N and 3O). Taken together, deficiency of Klf10 in CD4+ T cells increased IL-9, and ectopic delivery of IL-9 mediated the perivascular fibrosis phenotype, effectively phenocopying the perivascular fibrosis observed in Klf10-deficient CD4+ T cell mice. Table 3 is shown below.

TABLE 3
Parameters of cardiac physiology by echocardiography in Ang
II-infused Cre mice treated with or without recombinant mIL9

| | Cre + AngII + PBS group | Cre + AngII + IL9 group |
|---|---|---|
| Parameters | (n = 6) | (n = 6) |
| EF(Mean ± SD), % | 83.69 ± 5.94 | 87.14 ± 6.62 |
| FS(Mean ± SD), % | 53.27 ± 4.66 | 54.52 ± 4.94 |
| LV Mass(Mean ± SD), mg | 124.97 ± 11.07 | 121.65 ± 16.36 |
| LVEDD (Mean ± SD), mm | 2.73 ± 0.30 | 2.66 ± 0.15 |
| LVESD (Mean ± SD), mm | 1.44 ± 0.15 | 1.31 ± 0.29 |
| LVAW; d (Mean ± SD), mm | 1.42 ± 0.23 | 1.27 ± 0.05 |
| LVAW; s (Mean ± SD), mm | 1.96 ± 0.20 | 1.95 ± 0.11 |
| LVPW; d (Mean ± SD), mm | 1.09 ± 0.08 | 1.19 ± 0.07* |
| LVPW; s (Mean ± SD), mm | 1.81 ± 0.07 | 1.91 ± 0.08* |
| E/A (Mean ± SD) | 1.44 ± 0.29 | 1.48 ± 0.08 |
| E/e′ (Mean ± SD) | 30.28 ± 8.31 | 33.77 ± 6.99 |

EF, Ejection fraction; FS; fractional shortening; LV, Left ventricular; LVEDV/LVESV, LV end-diastolic/end-systolic volumes; LVAW; d/s, LV end-diastolic/systolic anterior wall thickness; LVPW; d/s LV end-diastolic/systolic posterior wall thickness.

### Example 4: KLF10 Binds to the IL-9 Promoter and Interacts with HDAC1 to Inhibit IL-9 Activation

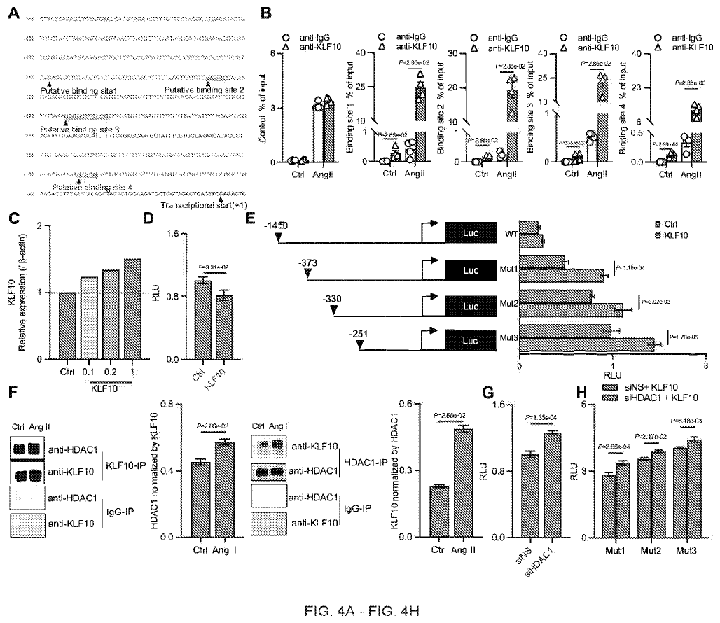

Previous studies have demonstrated that KLF10 could bind to target gene promoters to regulate transcription. Given the increased expression of IL-9 in TKO mice and in TKO CD4+ T cells, we explored potential underlying mechanisms of this regulation. As shown in FIG. 4A, there are four putative KLF10 DNA-binding sites in the 119 promoter sequence. Using chromatin immunoprecipitation (ChIP) assays with KLF10 antibodies in CD4+ T cells and the designated primer pairs directed for each putative binding site, we found that Ang II markedly increased the KLF10 enrichment to these sites (FIG. 4B). Overexpression of mouse KLF10 by transfecting overexpression plasmid in a heterologous HEK293T cell was confirmed by Western blot (FIG. 4C). It was shown that overexpression of mouse KLF10 in HEK293T cell significantly inhibited the transcriptional activity of a mouse IL9 promoter reporter (FIG. 4D). In addition, 5′ truncations of the IL-9 promoter demonstrated that successive deletions of the putative KLF10 binding sites increased transcriptional activity of the IL-9 promoter, suggesting that KLF10 mediates transcriptional repression (FIG. 4E). KLF10 has been reported to interact with HDAC1 to facilitate transcriptional repression. Consistently, we found that KLF10 recruited HDAC1 in T cells, especially after Ang II treatment (FIG. 4F). Next, we assessed the role of HDAC1 on IL-9 promoter in the presence of KLF10 by performing luciferase reporter assays (FIG. 4G). Depletion of HDAC1 with siRNAs demonstrated elevated luciferase activity among the WT and 3 mutant IL-9 promoters (FIGS. 4G and 4H). Collectively, these findings suggest that KLF10 interacts with HDAC1 and binds to the proximal IL-9 promoter to inhibit IL-9 transcriptional activity.

### Example 5: Transcriptomic Changes Involved in Ang II-Induced Perivascular Fibrosis

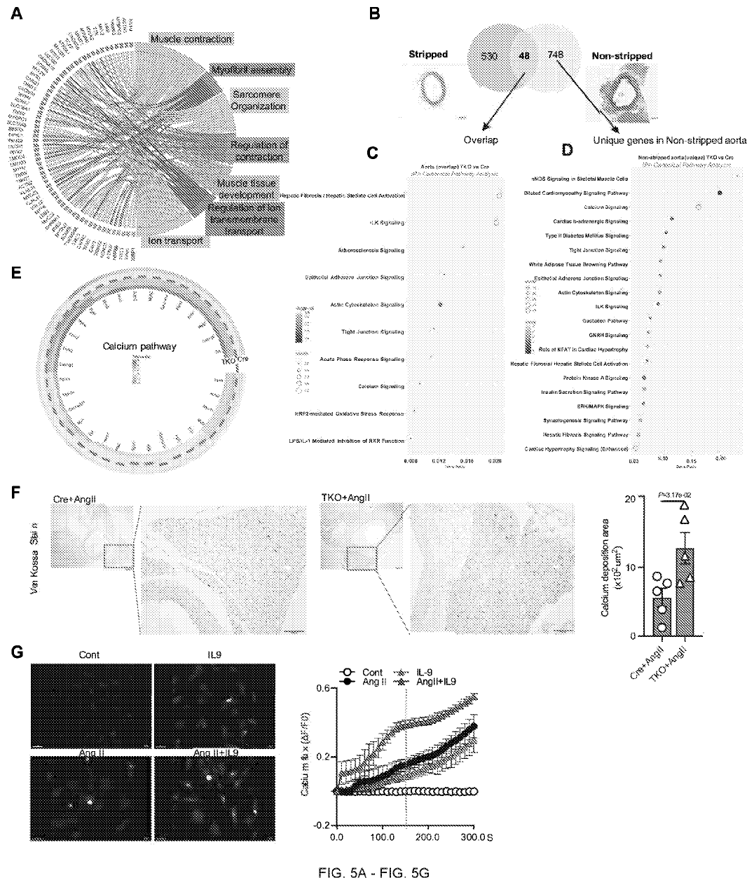

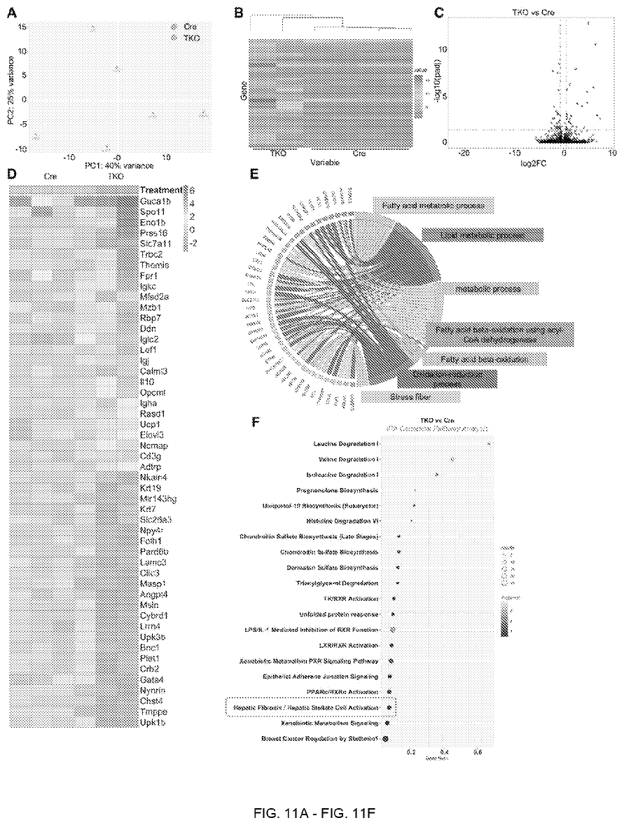

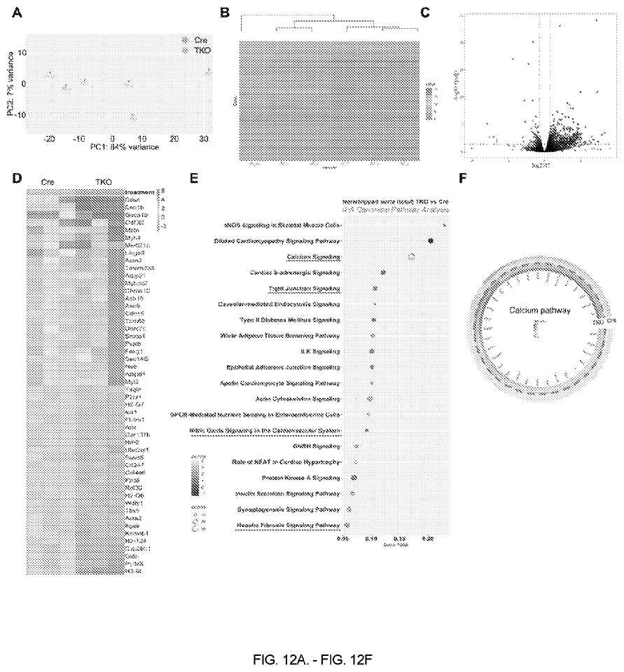

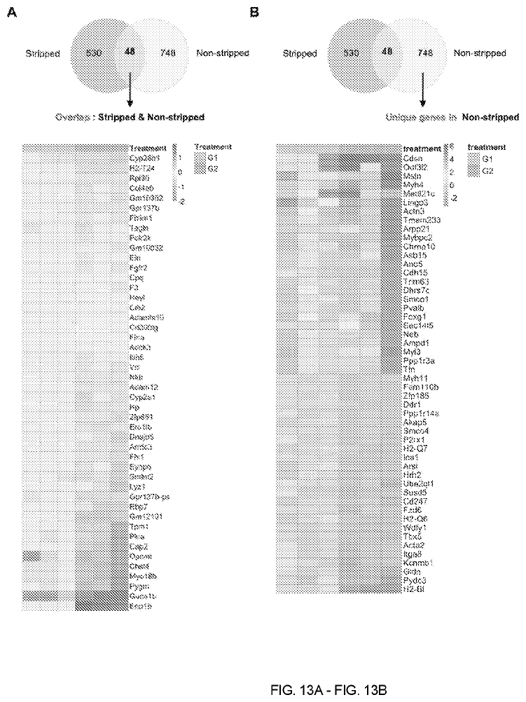

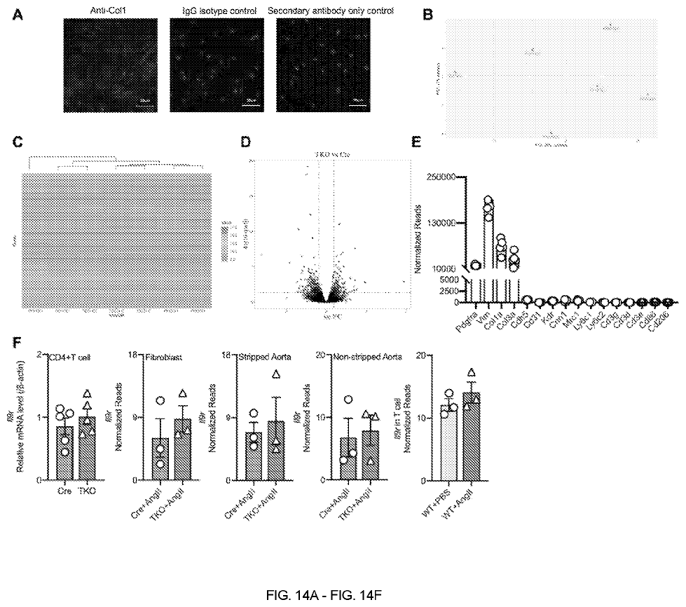

To ascertain the underlying global transcriptomic changes involved in perivascular fibrosis, we performed RNA-sequencing and gene ontology pathway analysis on stripped (perivascular adventitial tissue removed) (FIGS. 11A-11F) and non-stripped aorta (perivascular adventitial tissue remained intact) (FIGS. 12A-12F) from Cre or TKO mice after Ang II treatment. Comparison of stripped aortas between Cre and TKO mice revealed that most differentially regulated genes are related to metabolic processes but not to fibrotic process (FIGS. 11D through 11F). Considering the pathophysiological changes of fibrosis occurred in the perivascular adventitial tissue, we next focused our bioinformatic analysis on the non-stripped aortas. In contrast to Ang II-treated Cre mice, non-stripped aortas of TKO mice manifested pathways involved in assembly and muscle contraction (‘Muscle contraction’, ‘Myofibril assembly’), ion transport, and sarcomere organization (FIGS. 12A-12F and 5A). To identify transcriptomic differences derived from the vessel and adventitial tissues, we compared overlapping and unique DEGs (FDR <0.05) from the stripped and non-stripped aortic TKO vs Cre contrasts (FIGS. 5B, 13A, and 13B). From a total of 48 overlapping DEGs between TKO and Cre aortas, gene set enrichment analyses revealed several pathways involved in fibrosis, junctional signaling, and ion-related pathways (FIG. 5C). Notably, from the 748 unique DEGs derived from the non-stripped aorta, calcium signaling emerged as a dominant pathway in the TKO group (FIG. 5D) and the significantly regulated calcium signaling pathway genes were shown in the heatmap (FIG. 5E). As such, we performed von Kossa staining which revealed higher calcium deposition in the adventitial tissues from Ang II treated TKO mice compared to Cre controls (FIG. 5F). Furthermore, calcium flux assays in isolated aortic fibroblasts (FIGS. 14A through 14D) from C57BL/6 mice were performed to detect the effect of IL-9 on intracellular calcium levels. Consistent with previous reports, we found that Ang II as a pro-fibrotic agonist increased intracellular calcium; however, IL-9 further augmented this intracellular calcium flux in the presence of Ang II (FIG. 5G). Collectively, comparative RNA-seq transcript profiling identified several pathways associated with periadventitial remodeling and that the calcium signaling pathway is dominantly upregulated in non-stripped aortas of TKO mice after Ang II treatment. In addition, IL-9 and Ang II cooperatively increase calcium flux in aortic fibroblasts ex vivo.

### Example 6: TKO Fibroblasts Display an Activation Signature and IL-9 and Ang II Treatment Recapitulate the Phenotype in Control Fibroblasts

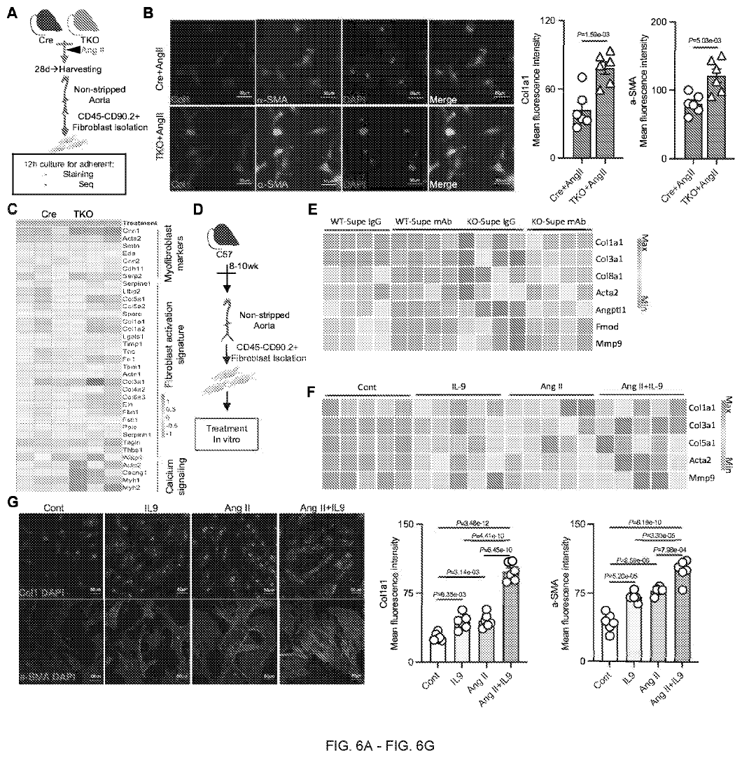

Given that calcium is involved in particularly critical signaling pathways for fibroblast and myofibroblast differentiation, we isolated and cultured aortic fibroblasts from Ang II-treated Cre control and TKO mice (FIG. 6A). Immunofluorescence staining showed that the expression of Coil a1 and α-SMA increased significantly in aortic fibroblasts of TKO mice compared with those from Cre mice (FIGS. 6B and 14A). RNA-sequencing of those TKO and Cre fibroblasts revealed upregulation of myofibroblast marker related genes (Cnn1, Acta2, etc.) and fibroblast activation signature genes (Col1a1, Fn1, etc.) in aortic fibroblasts from TKO mice (FIGS. 6C and 14B through 14E). Furthermore, calcium signaling related genes were also increased in TKO fibroblasts (FIG. 6C). To further assess whether these fibrotic related changes were mediated by IL-9, primary aortic fibroblasts were isolated from C57BL/6 mice and supernatants from WT or KO CD4+ T cells were added to the fibroblasts grown in culture with and without anti-IL-9 antibodies (FIG. 6D). Supernatants from KO CD4+ T cells increased the expression of multiple fibrotic genes including Col1a1, Col3a1, Col8a1, Acta2, Angptl1, Fmod, and Mmp9, whereas anti-IL-9 antibodies reversed their expression (FIG. 6E). Conversely, treatment of fibroblasts with recombinant IL-9 or Ang II increased the expression of Col1a1, Col3a1, Col5a1, Acta2, and Mmp9, and treatment with IL-9 plus Ang II together demonstrated additive effects (FIG. 6F). Immunofluorescence also confirmed that IL-9, Ang II, or IL-9 plus Ang II additively increased the expression of Col1a1 and α-SMA (FIG. 6G). Notably, there were no differences in expression of IL9r in any of the CD4+ T cell, fibroblast, or aortic tissue RNA-seq datasets (FIG. 14F). Altogether, fibroblasts from TKO mice exhibit an activated fibroblast signature along with myofibroblast-related genes, which is recapitulated in C57BL/6 fibroblasts treated with IL-9 and Ang II.

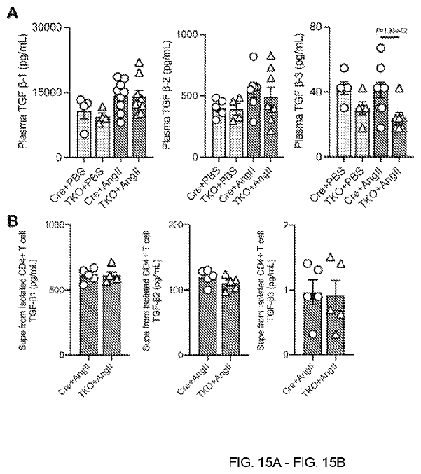

Considering KLF10 is a putative regulator of TGF-β signaling in specific disease states, we evaluated the expression of TGF-isoforms in plasma. There were no differences in TGF-β1 or TGF-β2 in plasma between KO and Cre control mice treated with Ang II (FIG. 15A). Despite a modest reduction in TGF-β3 in the plasma, there were no differences in expression levels for TGF-β1-3 between KO and Cre control CD4+ T cell supernatants (FIGS. 15A and 15B). In addition, examination for mediators of the TGF-β signaling pathway in our 3 RNA-seq datasets and 1 published T CD4+ cell dataset showed no impact of KLF10 on TGF-β signaling in vivo supported by pathway analyses from these 4 different RNA-seq datasets (Table 4). These findings suggest that the KLF10-mediated effects in CD4+ T cells on fibroblasts were likely independent of TGF-β signaling. Table 4 is shown below.

TABLE 4
TGF-β Signaling in different RNA-seq datasets by pathway analysis

| Dataset | P value | Ratio | Z-score | Molecules |
|---|---|---|---|---|
| Stripped Aorta | 0.49 | 0.0208 | / | ACVR1C, RASD1 |
| Non-stripped Aorta | 0.39 | 0.0312 | / | FOS, MAPK12, PITX2 |
| Aortic Fibroblast | 1 | 0.0104 | / | TGFB2 |
| Splenic T cell | 0.09 | 0.156 | 1.155 | ACVR1, ACVR1C, AMHR2, BMPR2, HNF4A, JUN, MAP2K3, |
| | | | | MAPK13, MRAS, RALB, RRAS2, SMAD7, SMURF1, TGFB3, VDR |

### Example 7: Single-Cell RNA Sequencing Revealed Fibroblast Heterogeneity and Activation Signatures Induced in TKO Aortas

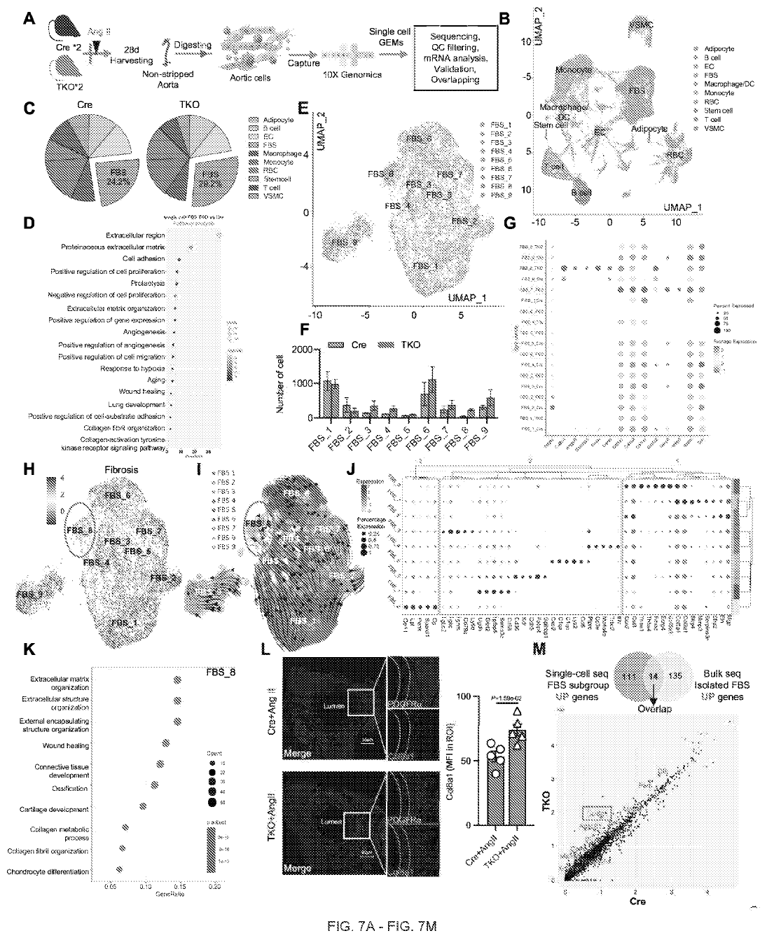

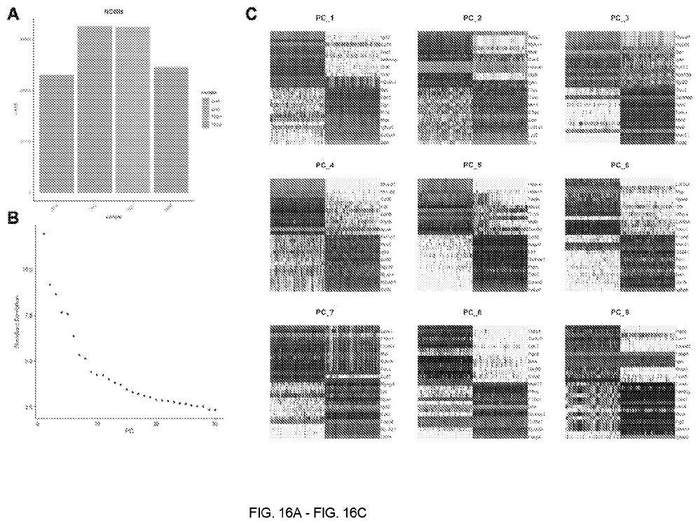

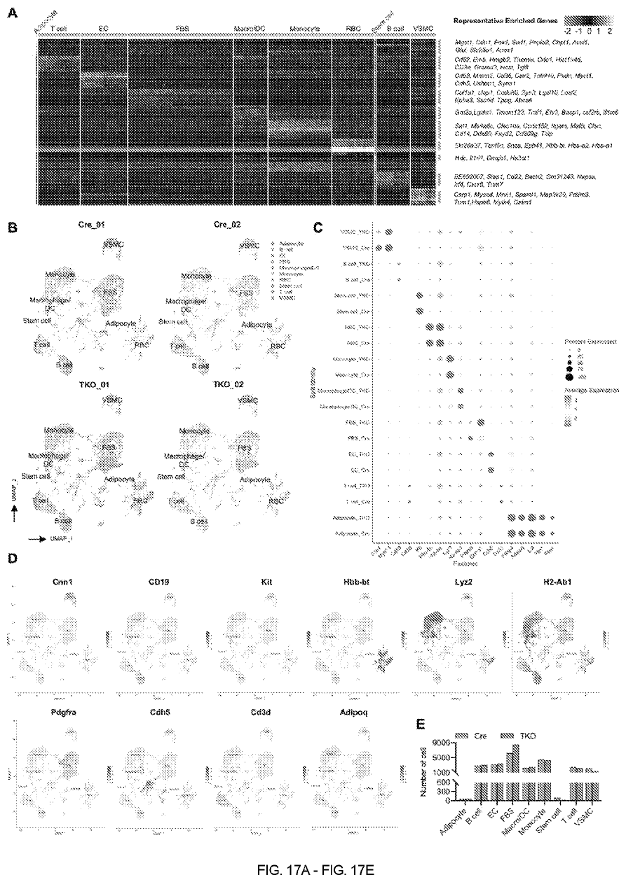

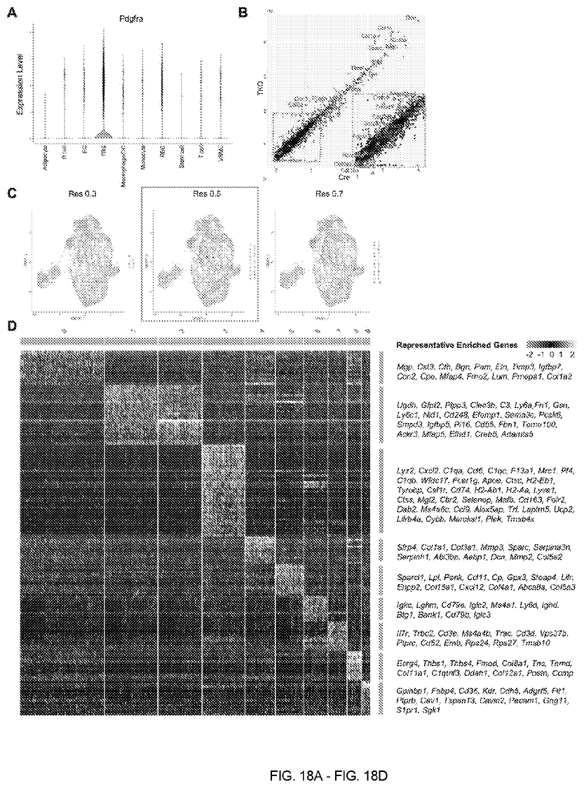

To further understand the differences in phenotypic heterogeneity in fibroblasts during the progression of perivascular fibrosis between TKO and control mice in response to Ang II treatment, we performed single-cell RNA-sequencing (scRNA-seq) of the descending aorta (FIG. 7A). A total of 53,346 cells from our four samples met quality control metrics (FIG. 16A) and were integrated and analyzed. The Seurat package was used for integration, clustering, and marker analysis (detailed in extended methods). We classified the cells in 10 major aortic cell types based on identified marker genes in each cluster (FIGS. 16B, 16C, 17A through 17D, and Table 5). The Uniform Manifold Approximation and Projection (UMAP) with the labeled clusters are shown in FIG. 7B and FIG. 17B. The percentage and the number of aortic cells from TKO and Cre aorta in each cell type are shown in FIGS. 7C, 17E, and Table 6. Considering the important biological role of fibroblasts in perivascular fibrosis, fibroblasts in the integrated dataset were identified by established markers such as platelet-derived growth factor receptor-a (Pdgfra) (FIG. 18A). Notably, we found that both the percentage and the number of fibroblasts increased in Ang II-treated TKO aortas compared to Cre controls (FIGS. 7C, 17E, and Table 6). Differential expression (DE) analysis between TKO and Cre aortas in the fibroblast clusters revealed a total of 627 differentially expressed genes (DEGs). Pathway enrichment analysis of these DEGs highlighted several pathways associated with fibrosis in the TKO aorta including extracellular matrix (ECM), ECM organization, and collagen fibril and extracellular fibril organization (FIG. 7D). Considering the important biological role of fibroblasts in the production of the enriched ECM, we assessed for relative changes in fibroblast activation signature genes, collagen family genes, and MMPs between TKO and Cre aorta controls, and found that many were upregulated in the Ang II treated TKO group (FIG. 18B). Tables 5 and 6 are shown below.

TABLE 5
Aortic cell type specific marker genes (top 30 for each cell type based on fold-change vs. all remaining clusters)

| Adipocyte | B cell | EC | FBS | Mac/DC | Monocyte | RBC | Stem cell | T cell | VSMC |
|---|---|---|---|---|---|---|---|---|---|
| Dbi | BE692007 | Cd93 | Tmem100 | Gm2a | Clec10a | Alas2 | Hdc | Cd52 | Csrp1 |
| Car3 | Stap1 | Mmrn2 | Uap1 | Lsp1 | Ccdc152 | Slc25a37 | Tpsb2 | Ms4a4b | Myocd |
| Hp | Cd22 | Cd36 | Ccdc80 | Srgn | Itgam | Tent5c | Cma1 | Cd3e | Mrvi1 |
| Mgst1 | Bank1 | Kdr | Fbn1 | H2-Eb1 | Mafb | Snca | Cpa3 | Tcf7 | Sparcl1 |
| Lpl | Ralgps2 | Ece1 | Rian | Plbd1 | Ctsc | Epb41 | Il1rl1 | Trbc2 | Map3k20 |
| Ghr | Cd79a | Kitl | Tnxb | Actb | Cd14 | Hbb-bt | Kit | Bcl11b | Pdlim3 |
| Fabp4 | Cd79b | Tspan7 | Lum | Cd74 | Folr2 | Hba-a2 | Mcpt4 | Ly6c2 | Tpm1 |
| Adipoq | Gm8369 | Cd300lg | Scara3 | Tmsb4x | Wwp1 | Hba-a1 | Mrgprb1 | Ccr4 | Palld |
| Cdo1 | H2-Ob | Clic5 | Thbd | H2-Ab1 | F13a1 | Hbb-bs | Slc18a2 | Igkv6-15 | Col18a1 |
| Cfd | Pax5 | Egfl7 | Col6a3 | Ccr2 | Cd83 | Car2 | Mlph | Gzmk | Rcan2 |
| Sncg | H2-Eb2 | Cyyr1 | Shc2 | H2-Aa | Ifi206 | Bpgm | Pde1c | Rps24 | Lpp |
| Pon1 | Il5ra | Flt1 | Abi3bp | Tyrobp | Pf4 | Gypa | Rasgef1c | Btg1 | Npnt |
| Angptl4 | Satb1 | Ly6c1 | Ddr2 | Mpeg1 | Ms4a6d | Slc4a1 | Erp27 | Rpl12 | Map1b |
| Pck1 | Rel | Adgrl4 | Dpep1 | S100a4 | Fcgr2b | Apol11b | 2700033N17Rik | Rplp1 | Mustn1 |
| Apoc1 | Fcer2a | Cav1 | Hmcn2 | Pim1 | Selenop | Hspa1b | Rasl10b | Rps15a | Myl9 |
| B3galt2 | Ighd | Sema6a | Gsn | Fau | Ms4a7 | Ctsd | Foxd2 | Rps27 | Fblim1 |
| Acvr1c | Cd37 | Etl4 | Ugp2 | Psap | Rnasel | Rsad2 | Vill | Rps8 | Itga8 |
| Scd1 | Sorl1 | Ptprb | Pthlh | H2-DMb1 | Cd33 | Gm42418 | 2610528A11Rik | Rplp2 | Ncam1 |
| Cidec | Gldn | Gpihbp1 | Col6a2 | Cbfa2t3 | Gpr34 | Ctsb | Fosb | Rps20 | Npy1r |
| Plin1 | Mmp15 | Klhl4 | Prrx1 | H2-DMa | Pltp | Bgn | Cybrd1 | Rpl23 | Adcy5 |
| Pnpla2 | Myc | Cldn5 | Gm26771 | Alox5ap | Frmd4b | Tuba1a | Stk32b | Rpl37a | Susd5 |
| Cyp2f2 | Mef2c | Adh1 | Cthrc1 | Btg1 | Cd300ld | Pam | Rhox8 | Tpt1 | Enah |
| Pdk4 | Il9r | Tm4sf1 | Svep1 | Tbc1d4 | C1qa | Apoe | Lif | Rpl41 | Nexn |
| Adig | Cdk5r1 | Tspan13 | Ndnf | Crip1 | Mrc1 | | Gm10101 | Rpsa | Tpm2 |
| Hcar1 | Cadm4 | Adgrf5 | Pdgfra | Ptprc | Hpgds | | Lilr4b | Rps28 | Pcdh7 |
| Cox8b | Col28a1 | Pecam1 | Nid1 | Lyz2 | Cd209f | | Adgrl3 | Rpl13 | Filip1l |
| Ces1d | Egr3 | Mgll | Smoc2 | Rpsa | Ninj1 | | Dnajb1 | Eef1a1 | Csrp2 |
| Clu | Il12a | Esam | Pcsk6 | Naaa | Cd68 | | Gfi1 | Cd3d | Sorbs2 |
| Gpd1 | Bhlhe41 | Sgk1 | Igf1 | H2afz | Fcna | | Hs3st1 | Rpl37 | Mylk |
| Arl4a | Kcna6 | Prex2 | Adamts5 | Rplp2 | Msr1 | | Gm26802 | Rpl38 | Igfbp7 |

TABLE 6
The number and the percentage of different aortic cell types

| | |
|---|---|
| Number | Percentage % |

| Cell type | Cre | TKO | Cre | TKO |
|---|---|---|---|---|
| Adipocyte | 62 | 65 | 0.24 | 0.22 |
| B cell | 2807 | 3073 | 10.93 | 10.51 |
| EC | 3146 | 3365 | 12.25 | 11.51 |
| FBS | 6221 | 8531 | 24.23 | 29.17 |
| Mac/DC | 2280 | 2415 | 8.88 | 8.26 |
| Monocyte | 4480 | 4332 | 17.45 | 14.81 |
| RBC | 2050 | 3792 | 7.98 | 12.97 |
| Stem cell | 94 | 27 | 0.37 | 0.09 |
| T cell | 2455 | 2204 | 9.56 | 7.54 |
| VSMC | 2080 | 1438 | 8.10 | 4.92 |

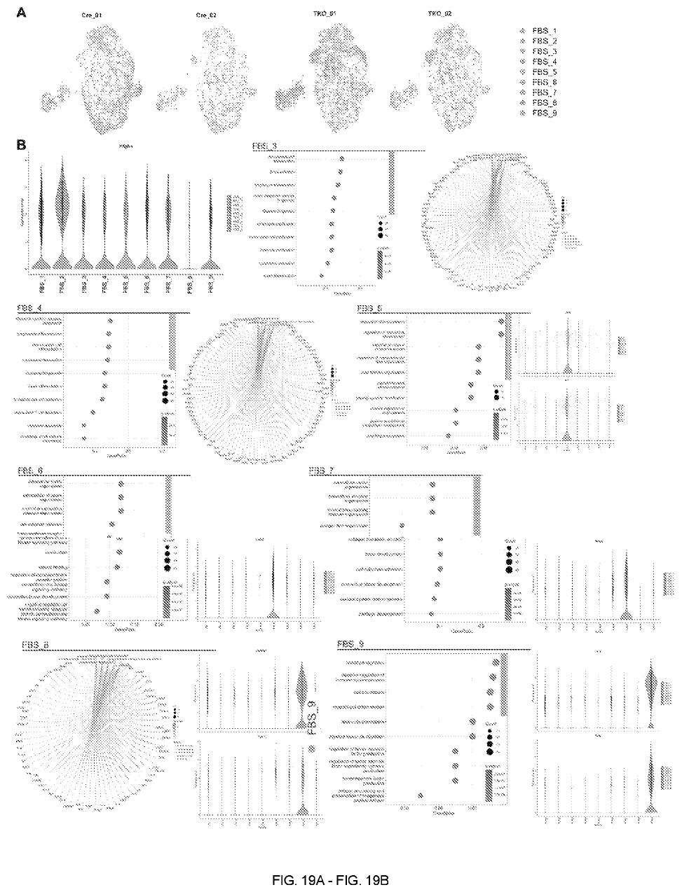

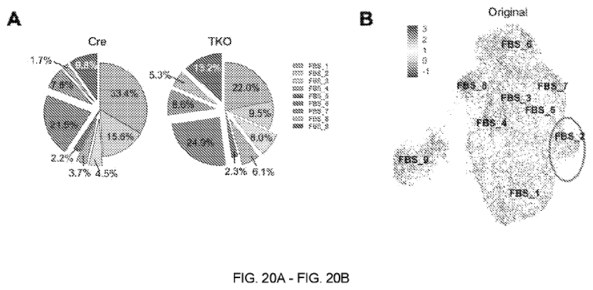

Further subsetting and cluster analysis was performed in the fibroblast group and 9 fibroblast subclusters (FBS) were identified based on the unique enriched markers (FIGS. 7E, 18C, 18D, 19A, 19B, and Table 7). Specifically, FBS_1 and FBS_2 showed high expression of Pdgfa, a canonical fibroblast marker that was found in perivascular regions rarely overlapping with αSMA; FBS_3 and FBS_4 were enriched in lymphocyte markers; FBS_5 was an EC-like fibroblast cluster that highly expressed Cdh5 and Kdr, suggesting potential endothelial-mesenchymal transition during fibrosis; FBS_6 highly expressed Col4a5, a collagen marker that was found to be related to a matrix fibroblast signature with endoplasmic reticulum stress (ERS) activation; FBS_7 highly expressed Mmps, also considered as matrix fibroblasts highly expressing Mmp3; FBS_8 highly expressed Col8a1, Col1a1, Angptl1, and Cthrc1, and was considered as a group of collagen-related pathological fibroblasts that are activated; and FBS_9 highly expressed Lyz2 and Cc/6, considered as a group of inflammation-related activated fibroblasts (FIGS. 19A and 19B). A differential abundance analysis for the conditions in each fibroblast subcluster showed a decrease in FBS_2 (log 2FC=−1.18, adj. pval=0.04), while there was an increase in the number of cells in FBS_8 (log 2FC=1.64, adj. pval=0.01) from the aorta of Ang II-treated TKO mice compared to Cre control (FIGS. 7F, 20A, and Table 8). Interestingly, the increased FBS subclusters (FBS_8) in TKO mice were highly associated with pathological or disease enriched fibrosis-related genes (FIG. 7G and Table 9). A combined gene score using a previously defined fibrosis-related gene list (Table 10) showed that FBS_8 had the highest score among all the FBS subclusters, suggesting the FBS_8 was a key fibrosis-driven subcluster (FIG. 7H). In contrast, a combined gene score using a group of canonical fibroblast genes (Table 10) showed that FBS_2 had the highest score, suggesting that FBS_2 may be the canonical type of fibroblast (FIG. 20B). Tables 7-10 are shown below.

TABLE 7
Fibroblast subgroups specific marker genes (top 30 for each
cell type based on fold-change vs. all remaining clusters)

| FBS_1 | FBS_2 | FBS_3 | FBS_4 | FBS_5 | FBS_6 | FBS_7 | FBS_8 | FBS_9 |
|---|---|---|---|---|---|---|---|---|
| Igfbp5 | Sparcl1 | Igkc | Il7r | Fabp4 | Mgp | Sfrp4 | Ecrg4 | Lyz2 |
| Sema3c | Lpl | Ighm | Trbc2 | Cd36 | Cst3 | Col1a1 | Thbs1 | Cxcl2 |
| Ugdh | Penk | Cd79a | Cd3e | Kdr | Cfh | Col3a1 | Thbs4 | C1qa |
| Gfpt2 | Ccl11 | Cd74 | Ms4a4b | Cdh5 | Bgn | Col1a2 | Fmod | Ccl6 |
| Cd55 | Cp | H2-Aa | Trac | Adgrf5 | Pam | Mmp3 | Col8a1 | C1qc |
| Smpd3 | Gpx3 | Iglc2 | Cd3d | Flt1 | Eln | Sparc | Tnc | F13a1 |
| Pi16 | Gsn | Ms4a1 | Vps37b | Ptprb | Timp3 | Serpina3n | Tnmd | Mrc1 |
| Cd248 | Steap4 | Ly6d | Ptprc | Cav1 | Igfbp7 | Lum | Col11a1 | Pf4 |
| Ackr3 | Lifr | Ighd | Cd52 | Tspan13 | Ccn2 | Serpinh1 | C1qtnf3 | C1qb |
| Pcsk6 | Enpp2 | H2-Ab1 | Emb | Cavin2 | Cpe | Abi3bp | Ddah1 | Wfdc17 |
| Adamts5 | Col15a1 | Btg1 | Rps24 | Pecam1 | Mfap4 | Bgn | Col12a1 | Fcer1g |
| Fbn1 | Cxcl12 | Bank1 | Btg1 | Gng11 | Fmo2 | Aebp1 | Col1a1 | Apoe |
| Creb5 | Col4a1 | Cd79b | Rps27 | S1pr1 | Lum | Dcn | Postn | Ctsc |
| Plpp3 | Abca8a | H2-Eb1 | Tmsb10 | Sgk1 | Pmepa1 | Mmp2 | Col1a2 | H2-Eb1 |
| Tmem100 | Col5a3 | Iglc3 | Rplp2 | Hspb1 | Col1a2 | Col5a2 | Comp | Tyrobp |
| Pla1a | C3 | Mef2c | Rps15a | mt-Cytb | Cd200 | Ctsk | Sparc | Csf1r |
| Efemp1 | Hsd11b1 | Fcer2a | Rps16 | Ly6c1 | Nr2f2 | Sfrp1 | Aspn | Cd74 |
| Efhd1 | Dpep1 | H2-Ob | Rpl12 | Tm4sf1 | Rbp1 | Timp1 | Rflnb | H2-Ab1 |
| Ly6c1 | Inmt | Rps27 | Rac2 | Sparcl1 | Col4a5 | Col14a1 | Tagln | H2-Aa |
| Adgrd1 | Col4a2 | Cd52 | Rps29 | Ets1 | Gdf10 | Fbln1 | Prelp | Lyve1 |
| Clec3b | Pdgfra | Pou2f2 | Rpl23 | Srgn | Itgbl1 | Postn | Bgr | Ctss |
| Uap1 | Srpx | Ccr7 | AW112010 | Tcf4 | Cped1 | Igf1 | Angptl1 | Mgl2 |
| Cd34 | Dcn | Rel | Gimap6 | Slfn5 | Gas6 | C4b | Slc7a2 | Cbr2 |
| Ly6a | Scn7a | Stk17b | Rps21 | mt-Atp8 | Lhfp | Adamts2 | Col5a2 | Selenop |
| Scara5 | Rarres2 | Rps24 | Rps13 | Aplp2 | Sfrp2 | Gas6 | Fxyd6 | Mafb |
| Axl | Acvr2a | Fau | Rpl37a | Cd200 | Fbln5 | Serpinf1 | Cthrc1 | Cd163 |
| Dpp4 | Tmem176b | Satb1 | Fau | Klf2 | Abccg | Cpxm1 | Ndnf | Folr2 |
| Pcolce2 | Col6a1 | Cxcr4 | Rpl32 | Slc6a6 | Nfib | Mfap4 | Serpine1 | Dab2 |
| Ddr2 | Ms4a4d | Malt1 | Rplp1 | Col4a2 | Sparc | Col5a1 | Fibin | Ms4a6c |
| Nid1 | Htra3 | Ptprc | Coro1a | Nol7 | Tm4sf1 | Igfbp7 | Ltbp2 | Fcgr2b |

TABLE 8
The number and the percentage of different fibroblast subgroups

| Fibroblast | Number | | | | Percentage % | | | |
|---|---|---|---|---|---|---|---|---|
| subgroup | Cre | Cre_total | TKO | TKO_total | Cre | TKO | logFC | FDR |
| FBS_1 | 1354806 | 2160 | 1121851 | 1972 | 33.4 | 22.0 | −0.56 | 0.31 |
| FBS_2 | 590171 | 1010 | 281147 | 850 | 15.6 | 9.5 | −1.18 | 0.04 |
| FBS_3 | 136152 | 288 | 491227 | 718 | 4.5 | 8.0 | 0.67 | 0.31 |
| FBS_4 | 117123 | 240 | 347202 | 549 | 3.7 | 6.1 | 0.60 | 0.31 |
| FBS_5 | 6678 | 144 | 11489 | 203 | 2.2 | 2.3 | −0.06 | 0.90 |
| FBS_6 | 1031357 | 1388 | 1490743 | 2233 | 21.5 | 24.9 | 0.29 | 0.60 |
| FBS_7 | 341152 | 493 | 506263 | 769 | 7.6 | 8.6 | 0.20 | 0.69 |
| FBS_8 | 6149 | 110 | 265210 | 475 | 1.7 | 5.3 | 1.64 | 0.01 |
| FBS_9 | 373264 | 637 | 820364 | 1184 | 9.8 | 13.2 | 0.33 | 0.60 |

TABLE 9
Differentially expressed fibrosis-related genes in FBS_8 in
Ang II treated-TKO group compared with Ang II treated-Cre group

FBS_8

| | Fold Change | p_val |
|---|---|---|
| Pdgfra | N/A | N/A |
| Col8a1 | 1.86 | 1.11E−07 |
| Angptl1 | 1.51 | 0.00050576 |
| Serpine1 | 2.15 | 5.36E−05 |
| Postn | 1.86 | 1.06E−07 |
| Comp | N/A | N/A |
| Col1a1 | 1.40 | 1.40E−05 |
| Col1a2 | N/A | N/A |
| Col3a1 | N/A | N/A |
| Col5a2 | 1.36 | 0.001580595 |
| Mmp2 | 0.83 | 0.47076991 |
| Mmp3 | N/A | N/A |
| Sparc | N/A | N/A |
| Dcn | 0.79 | 0.4572286 |

TABLE 10
The list of canonical fibroblast genes and fibrosis
related genes for the add module score

| Canonical fibroblast | Fibrosis related |
|---|---|
| genes | genes |
| Pdgfra | Col1a1 |
| Col4a1 | Col1a2 |
| Fn1 | Col3a1 |
| Gsn | Col8a1 |
| Col15a1 | Mmp2 |
| | Mmp3 |
| | Serpine1 |
| | Sparc |
| | Lgals1 |
| | Angptl1 |
| | Timp1 |
| | Timp3 |
| | Postn |
| | Comp |
| | Acta2 |

We next performed RNA velocity analysis, a method which calculates both spliced and unspliced mRNA counts to predict potential future directionality and speed of cell state transitions. Higher proportions of unspliced to spliced mRNA highlights the active transcription state within a given cell cluster. Using this analysis, we obtained a trajectory of the different FBS subclusters states (FIG. 7I). FBS_2 appeared to be the initial state of FBS in the stream plot (FIG. 7I), which was consistent with our previous observation on the enriched markers of canonical FBS genes in FBS_2. Following the paths from FBS_2 in the stream plot, we observed FBS_8 as the most progressed state from FBS_2 (FIG. 7I).

Together with the observation of a higher percentage of cells in FBS_8 in TKO samples and fibrosis-related gene markers for this subcluster, FBS_8 is likely the most important cluster contributing to the perivascular fibrosis in TKO mice. Furthermore, k-means cluster analyses confirmed FBS_8 is completely different with FBS_1-5 and FBS_9 (FIG. 7J). Further pathway analysis found that FBS_8 is highly involved in extracellular matrix and structure organization (FIG. 7K). Since Col8a1 is one of the unique collagen-related genes in FBS_8, we assessed its expression by co-staining with PDGFRa in the aorta. The immunofluorescence staining result showed that Col8a1+PDGFRa+ cells were located in the perivascular area and the expression of Col8a1 increased in Ang II treated-TKO group (FIG. 7L). Interestingly, we also found that Col8a1 increased in the aortic fibroblasts from TKO mice after overlapping transcripts between the fibroblast upregulated DEGs from the scRNA-seq dataset and the upregulated DEGs from the isolated fibroblast bulk RNA-seq dataset (FIG. 7M). Taken together, scRNA-seq of aortas from Cre and TKO mice after Ang II treatment revealed fibroblast heterogeneity with activated signatures most prominent among FBS_8, and induction of pathways associated with robust ECM and perivascular fibrosis.

### Example 8: Neutralization of Endogenous IL-9 Reversed the Ang II-Induced Perivascular Fibrosis and Ameliorated Injury of Hypertension-Related Organs

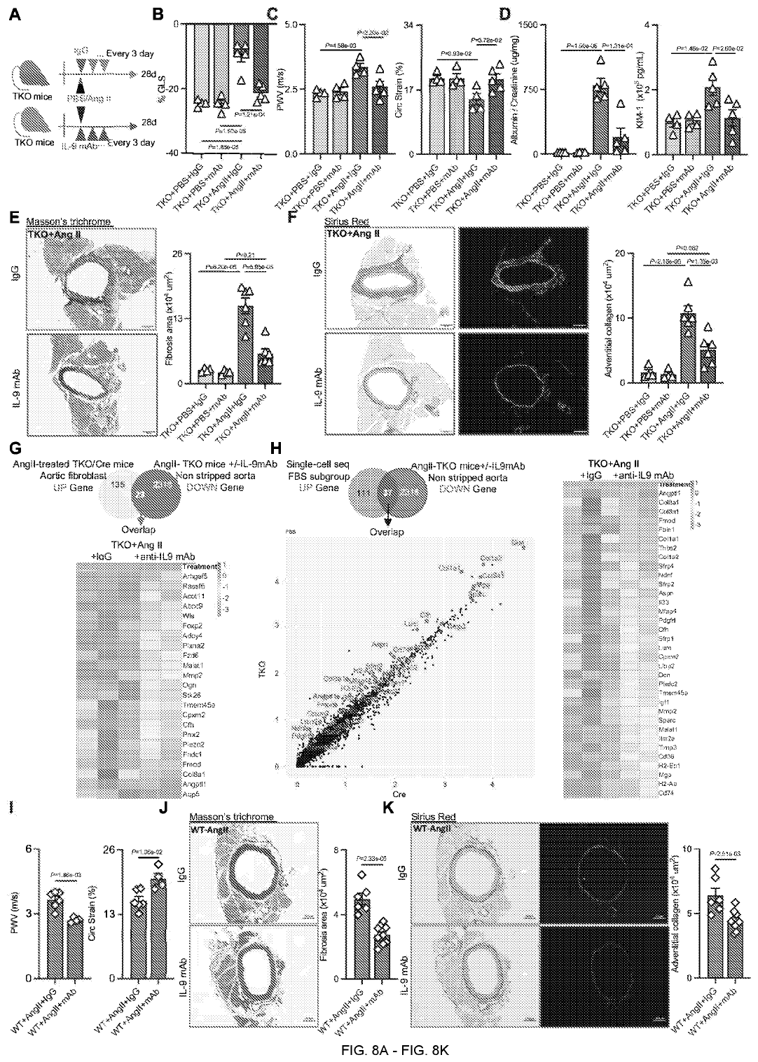

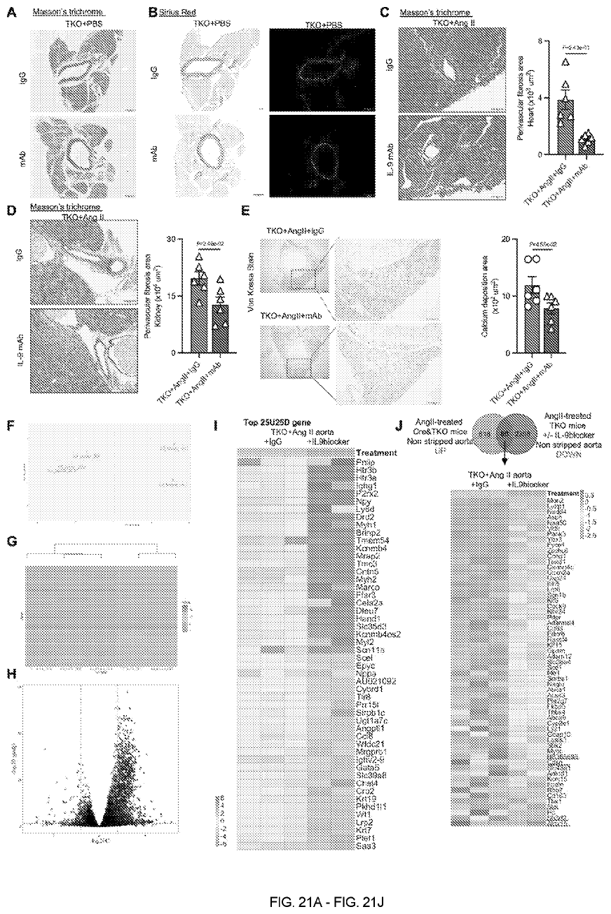

Considering the crucial profibrotic role of IL-9 in Ang II-treated perivascular fibrosis and the elevated plasma levels observed in the TKO mice, we hypothesized that perivascular fibrosis might be rescued by administration of anti-IL-9 neutralizing monoclonal antibodies (mAbs) in Ang II-treated TKO mice (FIG. 8A). TKO mice treated with anti-IL9 mAbs showed improved LV GLS (FIG. 8B, Table 11), decreased PWV, and increased Circ Strain (FIG. 8C) compared with the IgG treated Ang II group, while no difference was observed in PBS groups. In addition, the ratio of albumin/creatinine and the value of KIM-1 decreased after treatment with IL-9 mAb in Ang II group (FIG. 8D). Furthermore, histopathology analyses revealed significantly decreased perivascular fibrosis in the aorta, heart, and kidney in the anti-IL-9 mAb treatment group, and reduced aortic adventitial collagen was also observed in the anti-IL-9 mAb treated Ang II group compared with the IgG treated Ang II group (FIGS. 8E, 8F, and 21A through 21D). Moreover, the increased calcium deposition in the perivascular adventitia was reversed by treatment with anti-IL-9 mAb in Ang II-treated TKO mice (FIG. 21E). Table 11 is shown below.

TABLE 11
Parameters of cardiac physiology by echocardiography in Ang
II- infused TKO mice treated with or without anti-IL9 mAb.

| | TKO + AngII + IgG group | TKO + AngII + IL9mAb group |
|---|---|---|
| Parameters | (n = 6) | (n = 6) |
| EF(Mean ± SD), % | 80.43 ± 8.88 | 82.67 ± 6.48 |
| FS(Mean ± SD), % | 57.16 ± 6.20 | 58.87 ± 4.95 |
| LV Mass(Mean ± SD), mg | 125.35 ± 12.88 | 118.15 ± 22.95 |
| LVEDD (Mean ± SD), mm | 2.54 ± 0.31 | 2.67 ± 0.27 |
| LVESD (Mean ± SD), mm | 1.14 ± 0.18 | 1.17 ± 0.08 |
| LVAW; d (Mean ± SD), mm | 1.38 ± 0.18 | 1.19 ± 0.10* |
| LVAW; s (Mean ± SD), mm | 1.20 ± 0.19 | 1.18 ± 0.19 |
| LVPW; d (Mean ± SD), mm | 1.21 ± 0.15 | 1.02 ± 0.13* |
| LVPW; s (Mean ± SD), mm | 1.78 ± 0.18 | 1.56 ± 0.13* |
| E/A (Mean ± SD) | 1.36 ± 0.25 | 1.61 ± 0.52 |
| E/e′ (Mean ± SD) | 32.93 ± 5.85 | 22.24 ± 3.63** |

EF, Ejection fraction; FS; fractional shortening; LV, Left ventricular; LVEDV/LVESV, LV end-diastolic/end-systolic volumes; LVAW;d/s, LV end-diastolic/systolic anterior wall thickness; LVPW;d/s LV end-diastolic/systolic posterior wall thickness.

To further understand the transcriptomic changes associated with the rescue of perivascular fibrosis with anti-IL-9 mAbs, RNA-seq differential expression analysis was performed from the anti-IL9 mAbs vs IgG groups in non-stripped aortas from TKO mice after Ang II treatment (FIGS. 21F through 21I). By analyzing the overlap between upregulated genes in non-stripped aortas from Ang II-treated TKO vs Cre mice with the downregulated transcripts in the anti-IL-9 mAb-treated TKO aortas, we identified potential genes involved with reversal of perivascular fibrosis, including Alox15 and Haptoglobin (Hp) (FIG. 21J). We also compared the significant downregulated transcripts from the non-stripped aortic anti-IL-9 mAb treatment with the significant upregulated transcripts from aortic fibroblasts of Ang II treated TKO vs. Cre mice to identity transcripts uniquely changed in fibroblasts after anti-IL-9 mAb rescue (FIG. 8G). Indeed, the profibrotic genes Col8a1, Mmp2, Fmod, and Angptl1, which were identified as significantly increased in TKO aortas and in isolated fibroblasts by both bulk RNA seq and scRNA seq (FIG. 7M), were all decreased after treatment with anti-IL-9 mAbs (FIG. 8G). In addition, these genes were observed as overlapping from the upregulated genes from scRNA-seq with the downregulated genes from anti-IL-9 mAb RNA-seq dataset (FIG. 8H).

To further determine if anti-IL-9 mAbs could provide therapeutic benefit outside of the context of genetically modified mice, we treated C57BL/6 mice with Ang II and with or without anti-IL-9 mAbs. Treatment with anti-IL9 mAbs showed decreased PWV and increased circumferential strain compared with the IgG treated control group (FIG. 81). Furthermore, histopathology analyses revealed significantly decreased perivascular fibrosis and reduced aortic adventitial collagen in the aorta in the anti-IL-9 mAb treatment group compared with the IgG control group (FIGS. 8J-8K).

Collectively, neutralization of endogenous IL-9 ameliorated the Ang II-induced dysfunction of hypertension-related organs and reversed perivascular fibrosis not only in TKO mice by suppressing the expression of profibrotic genes, but also in C57BL/6 mice.

### DISCUSSION

The molecular events in T cells specifically driving perivascular fibrosis in response to Ang II remain poorly understood. In this study, we found that Ang II induced Kif10 expression in CD4+ T cells in vitro and in vivo, indicating involvement of KLF10 in Ang II mediated end organ damage and fibrosis. We found that Ang II treated TKO mice developed adverse cardiac remodeling reflected by impaired global longitudinal strain (GLS), worse arterial stiffness (evaluated by PWV and circumferential strain), and more kidney injury (evaluated by the levels of albuminuria and KIM-1 expression). Notably, the TKO mice exhibited marked perivascular fibrosis in hearts, aortas, and kidneys. However, we found that those functional and histopathological changes were independent of blood pressure. These findings build upon a broader role for KLF10 in CD4+ T cells in a range of chronic inflammatory disease states including atherosclerosis, insulin resistance, and fatty liver disease.

Surprisingly, we did not find any differences in cardiac hypertrophy or interstitial fibrosis between Ang II-induced TKO and Cre control mice. Considering cardiac hypertrophy results from sustained pressure overload or heart injury, the lack of difference in blood pressure is consistent with the findings of similar extent of cardiac hypertrophy between TKO and Cre mice. The lack of difference in blood pressure between TKO and Cre also allowed us to leverage novel molecular insights into perivascular fibrosis.

Ang II treatment increased the expression of the profibrotic cytokine IL-9 in both groups. Surprisingly, IL-9 was the only cytokine detected much higher in the TKO mice after Ang II treatment compared with Cre mice, which suggested that IL-9 played a crucial role in this perivascular fibrosis phenotype. IL-9 was reported in the regulation of immune responses and involved in the pathogenesis of various inflammatory diseases, including a profibrotic role in lung inflammation and fibrosis, liver fibrosis, and kidney fibrosis. In cardiovascular disease, IL-9 has been implicated in the pathogenesis of atherosclerosis (exerting pro-atherosclerotic effects) and in heart failure (aggravating isoproterenol-induced heart failure). To confirm the profibrotic role for IL-9 in Ang II-induced perivascular fibrosis, we systemically administrated recombinant murine IL-9 in Ang II-treated Cre mice and found that it increased perivascular fibrosis and induced dysfunction in heart, kidney, and aortas, which effectively phenocopied that observed in Ang II-treated TKO mice. Importantly, both the end organ injury and perivascular fibrosis were rescued by administration of anti-IL-9 neutralizing monoclonal antibodies in Ang II-treated TKO mice. Taken together, the data provided herein demonstrate that blocking IL-9 is an attractive therapeutic strategy for treatment of perivascular fibrosis or hypertensive-associated end organ damage.

The main cellular sources of IL-9 are T cell subsets, including Th2, Treg, and from recently identified Th9 cells. Consistently, flow cytometry also found that the percentage of CD4+IL9+ and CD4+IL4+ T cells increased in the aortic tissue and spleen in Ang II-infused TKO mice compared with Cre mice, while there were no differences in CD4+IL17+, CD4+FoxP3+, or CD4+IFNW|+ T cells. Although the percentage of CD4+IL4+ cells increased in TKO mice, there were no differences of released IL-13, IL-5, or IL-4 in both male and female TKO mice compared with Cre controls. In addition, using flow cytometry we found there was no difference in the percentage of CD8+IL-9+ cells between Ang II treated Cre and TKO mice in both aorta and spleen. Furthermore, we found increased transcript and protein expression of IL-9 in both isolated CD4+ T cells from Ang II-infused TKO mice in vivo and Ang II-treated primary TKO CD4+ T cells in vitro, which suggested that KLF10 may directly regulate IL-9 expression in CD4+ T cells. Unlike many other transcription factors, KLF10 is primarily known to repress transcription of targeted genes. For example, KLF10 can negatively regulate cardiac MCP-1 expression by blinding to the MCP-1 promoter with histonedeacetylase 1 (HDAC1). Moreover, KLF10 can reduce acetylated histone H4 on the C/EBPα promoter and inactivate C/EBPα transcription. In our study, we found that KLF10 can bind to the IL-9 promoter and interact with HDAC1 to inhibit 119 transcription. These findings suggest that the Ang II-mediated increase in KLF10 expression in CD4+ T cells may serve a protective, counter-regulatory mechanism to limit perivascular fibrosis. However, this increase is likely not sufficient to inhibit IL-9 release completely and can still trigger perivascular fibrosis. As a pro-fibrotic cytokine, IL-9 could also be regulated by multiple factors. As a tonic repressor of IL-9, KLF10 once deleted in CD4+ T cells triggers more IL-9 production and subsequently perivascular fibrosis.

Ang II treatment, as a pathological stress, increases intracellular calcium to stimulate fibroblasts to differentiate into myofibroblasts, which can further induce fibrosis by secreted extracellular matrix (ECM) proteins, matrix metalloproteinases (MMPs), and others. In our study, we found that fibrosis related signaling, ECM related pathways, and calcium signaling were particularly upregulated in Ang II-treated TKO mice compared with Cre control mice by RNA-seq. Given that calcium homeostasis is important in pulmonary fibroblasts and cardiac fibroblasts, we isolated aortic fibroblasts and found myofibroblast markers, fibroblast activation signature genes, and calcium signaling genes to be highly expressed from Ang II-treated TKO fibroblasts by RNA-seq. In particular, we showed for the first time that IL-9 can also stimulate increased calcium flux and fibrosis phenotypes in isolated primary fibroblasts under basal and Ang II treatment. Thus, the IL-9 mediated perivascular fibrosis may be dependent in part on hyperactivation of calcium signaling in fibroblasts.

Fibroblasts are important cells involved in the maintenance of tissue integrity and tissue repair in response to injury. They can differentiate into different phenotypes including an ECM-producing contractile phenotype that further contributes to the secretion and accumulation of ECM and MMPs leading to the progression of fibrosis in a range of fibrotic diseases. By using scRNA-seq, TKO aortic fibroblasts subsets exhibited higher activation markers including Col1a1, Col8a1, and Col1a2. Furthermore, nine identified subpopulations of fibroblasts displayed different aspects of fibroblast activation in TKO aortas, which demonstrated their relevance to perivascular fibrosis. In addition, in the Ang II-treated TKO aortas, the percentage of fibroblasts increased and the number and the percentages of specific FBS clusters were more abundant especially FBS_8. FBS_8 shows high expression of collagen-related genes including: Cthrc1 (Collagen triple helix repeat containing 1), a potential marker for activated fibroblasts in the heart and lungs; Tnc (Tenascin-C), a marker involved in stimulating collagen-related gene expression, myofibroblast transformation, and perivascular inflammation and fibrosis; and Ddah1, a marker related to perivascular or adventitial fibrosis and vascular remodeling. Using RNA velocity analyses, FBS_8 represented one of the most advanced fibroblast clusters, while FBS_2 appeared as an earlier state reflecting less activation. Indeed, our results showed that FBS_8 is highly involved in the extracellular matrix and structure organization pathway and highly enriched for fibrosis-related genes, whereas FBS_2 was enriched for most of the canonical fibroblast genes. Col8a1 was found to be a unique gene in FBS 8, and Col8a1+PDGFRa+ cells were located in the perivascular area and the expression of Col8a1 increased in the Ang II treated-TKO group. Thus, we consider the FBS_8 subcluster as the key fibroblast subcluster contributing to the perivascular fibrosis in the Ang II treated-TKO group. In addition, the majority of the other increased genes in the TKO group are known to contribute to the development of fibrosis, including Col1a1, Mmp2, Angptl1, Comp, Fmod, and Acta2-all decreased after administration of anti-IL-9 mAbs. Collectively, this anti-IL-9 therapeutic strategy demonstrated reduced end-organ injury and perivascular fibrosis in part by downregulating these fibrosis-related gene signatures.

In summary, KLF10 is upregulated in PBMCs of hypertensive patients and in peripheral CD3+ T cells and CD4+ T cells in Ang II-treated mice. Administration of Ang II in TKO mice triggered perivascular fibrosis, multi-organ dysfunction in heart, kidney, and aorta, and release of IL-9 from CD4+ T cells. These functional and histopathological differences were independent of blood pressure between Ang II-treated TKO and Cre mice. Mechanistically, in response to Ang II treatment, KLF10 bound to the IL-9 promoter and interacted with HDAC1 to inhibit IL-9 transcription. Ectopic IL-9 activated calcium flux, induced fibroblast activation and differentiation, increased production of collagen and ECM, thereby promoting the progression of perivascular fibrosis and inducing target organ dysfunction. Importantly, IL-9 neutralizing antibodies potently rescued perivascular fibrosis, and target organ dysfunction in Ang II treated TKO and C57BL/6 mice. RNA-seq and scRNA-seq revealed the presence of fibroblast heterogeneity and marked myofibroblast activation in non-stripped aortas from Ang II-treated TKO mice. These results indicate that the KLF10-IL-9 signaling axis in CD4+ Tcells tightly regulate the processes of Ang II-induced pathological perivascular fibrosis and end organ damage and provide new therapeutic opportunities for the treatment of hypertensive-associated disease.

### Example 9: Materials and Methods

### Animal Studies

C57BL/6 mice were obtained from the Charles River Laboratories (Charles River, MA). CD4-specific KLF10 knockout mice (Klf10^{fl/fl}/CD4^{Cre+}, referred to as TKO) were generated by crossing Klf10^{fl/fl} mice with CD4-transgenic Cre (Klf10^{+/+}CD4^{Cre+} referred to as Cre) as we described in Wara et al. Cell Rep. 33:108550 (2020), and we used littermate Cre as the control. All animal procedures were performed in accordance with the Institutional Animal Care and Use Committee at Harvard Medical School (#2016N000182) and the National Institutes of Health Guide for Care and Use of Laboratory Animals.

All mice used were age- and sex-matched in all experiments. Angiotensin II (Ang II) osmotic mini-pump chronic infusion was performed as previously described in Nosalski et al. Circ. Res. 126:988-1003 (2020) and Cambier et al. Hypertension. 72:370-380 (2018). In brief, mice were anesthetized with isoflurane and osmotic mini pumps (ALZET model 2004, model 2006, CA, USA) releasing Ang II (1000 ng/kg/min, A9525, Sigma-Aldrich, MO, USA) were implanted subcutaneously where they remained for 28 days or 42 days. Sham animals were infused with PBS solution.

In some experiments, recombinant mIL9 or anti-IL-9 mAbs were used for administration. To elucidate the effect of IL-9 in Ang II-induced perivascular fibrosis, Cre mice were randomly divided into two groups: the control group (Cre+AngII+PBS) and the recombinant IL-9 treatment group (Cre+AngII+mIL-9) were injected intraperitoneally (i.p.) with 0.2 ml PBS containing 1% BSA or 200 ng rIL-9 (409-ML, R&D Systems, MN, USA) dissolved in 0.2 ml PBS containing 1% BSA daily for 28 days. For the IL-9 neutralization study, TKO mice were randomly divided into four groups: isotype control antibody groups (TKO+PBS+IgG, and TKO+AngII+IgG) injected i.p. with 100 μg mouse isotype control mAb (BE0085, Bio X Cell, NH, USA), and the anti-IL-9 antibody treatment groups (TKO+PBS+mAb, and TKO+AngII+mAb) injected i.p. with anti-mouse IL-9 neutralizing mAbs (BE0181, BIO X CELL, NH, USA). Timelines of each experiment were described in FIGS. 1E, 2A, 3F, 3H, 3J, 6A, 7A, and 8A. At the end of in vivo experiments, the mice were euthanized with CO2 gas.

### Echocardiography

Echocardiography and aortic imaging were performed by using a high-resolution, high-contrast ultrasound imaging system Vevo 3200 (VisualSonics, FUJIFILM, Toronto, Canada). Briefly, mice were anesthetized with light (˜1%) isoflurane until the heart rate stabilized to 400 to 500 beats per minute. Parasternal long-axis images were acquired in B-mode with appropriate position of the scan head to identify the maximum LV length. Long axis views were used for the analysis of global longitudinal strain (GLS) of the left ventricle. For the aortic imaging, using the MX550D transducer, transverse and longitudinal B-mode images of the abdominal aorta were obtained from the level of the diaphragm to the renal arteries to measure the maximal aortic diameter. To measure circumferential strain, M-mode images were obtained longitudinally at three points along the abdominal aorta, and circumferential strain was calculated by averaging across the 3 sampled segments of the aorta. Circumferential strain (Circ strain) was calculated using the following formula: circumferential strain=(IDs−IDd)/IDd×100, where IDs=internal aortic diameter at systole, IDd=internal aortic diameter at diastole. Pulse wave velocity (PWV) was indirectly obtained by measuring the time from ventricular contraction (R wave on electrocardiography) to onset of the pulse wave (assessed by pulse wave doppler) at 2 points along the abdominal aorta—point 1 just distal to the diaphragm and point 2 just proximal to the ostium of the right renal artery. Using these measurements, PWV was calculated by the formula: PWV=L/(T1−T2), where L is the length of the abdominal aortic segment between the two points sampled, T1 is the time from R wave to pulse wave onset at point 1 (proximal abdominal aorta) and T2 is the time from R wave to pulse wave onset at point 2 (distal abdominal aorta).

### Aortic Blood Pressure Measurement

Mice were anaesthetized with isoflurane and implanted with a radiotelemetry transmitter (TA11 PA-C20, DSI, MN, USA) into the left carotid artery as described previously in Mirabito et al. Hypertension. 64:626-631 (2014). The basal mean arterial pressure (MAP) was directly measured before harvesting and recorded by LabChart.

### Cytokine Profiling in the Plasma and Supernatants

Mouse blood samples were obtained by cardiac puncture during sacrifice, then centrifuged for separate the plasma. For the supernatants from primary CD4+ T cells, 1*10\hat{ }6 isolated cells per well were plated in the 24-well plate with Mouse T-Activator CD3/CD28 for T-Cell expansion and activation (11452D, Gibco, MA, USA) in 1 mL RPMI Media 1640 with 10% FBS and 1% P/S according to the manufacturer's instructions. After culture for 24 h, the supernatants were collected and directly subjected for the further experiments. Plasma and supernatants from primary CD4+ T cells then subjected to Mouse Cytokine/Chemokine 31-Plex Discovery Assay® Array (MD31), and TGFβ 3-Plex Discovery Assay® Multi Species Array (TGFβ1-3) (Eve Technologies, AB, Canada).

### Myography

Isometric tension studies of mesenteric arterioles were performed using 2-mm segments of third-order mouse mesenteric arterioles dissected free of perivascular fat. Studies were performed in a small vessel horizontal wire myograph (Radnoti M1000) containing a physiological salt solution composed of 130 mM NaCl, 4.7 mM KCl, 1.2 mM MgSO4, 1.2 mM KH2PO4, 25 mM NaHCO₃, 5 mM glucose, and 1.6 mM CaCl2). The isometric tone was recorded for each vessel using LabChart Pro v7.3.7 (AD Instruments). The vessels were equilibrated over a 20-minute period at 37° C. A passive circumference-tension curve was generated for each vessel to determine optimum passive tension to simulate an in vivo transmural pressure of 100 mmHg according as previously described in del Campo et al. Methods Mol. Biol. 1339:255-276 (2015). After normalization, vessels were contracted with 60 mM KCl to assess the integrity of the vessel, and then endothelium-independent vascular relaxation was tested using increasing concentrations of sodium nitroprusside after preconstriction with norepinephrine (10 μM). To test smooth muscle contractility, vessels were treated with increasing concentrations of phenylephrine.

### Measurements of Urinary Albumin, Creatinine, and Kidney Injury Molecule 1 (KIM-1)

Mouse urine was collected after 4 weeks of Ang II infusion and subjected to ELISA using Mouse Albumin ELISA Kit (ab207620, Abcam, MA, USA), Creatinine Assay Kit (ab65340, Abcam, MA, USA), and Mouse KIM-1 ELISA Kit (TIM1) (ab213477, Abcam, MA, USA). Albumin concentration was divided by creatinine concentration in each sample to determine the albumin/creatinine ratio.

### Histological Analysis

Mice were perfused with cold PBS, and the non-stripped aortas, hearts, and kidneys were harvested. The tissues were fixed in 4% paraformaldehyde (PFA), embedded in paraffin, and sectioned.

Serial sections were stained with Hematoxylin and eosin (H&E) for recognizing various tissue types and the morphologic changes, Masson's trichrome for detection of fibrosis, Sirius Red for observation of fibrosis level and collagen, and von Kossa (ab150687, Abcam, MA, USA) for visualization of calcium deposits as previously described in Ni et al. Arterioscler. Thromb. Vasc. Biol. 41:2399-2416 (2021) or following the instructions provided by the manufacturer. Images were acquired using a Nikon Eclipse microscope (NY, USA), and the staining area was measured using computer-assisted image quantification (Image-Pro Plus software, Media Cybernetics, Inc., Rockville, MD, USA).

Cardiac sections were also stained with ALEXA FLUOR®488 conjugated wheat germ agglutinin (WGA) (W11261, Invitrogen, MA, USA) for measurement of cardiomyocyte myocyte cross-sectional area and size according to the manufacturer's instructions. For tissue immunofluorescence staining, antibodies including anti-KLF10 (PA5-38674, Invitrogen, MA, USA), anti-IL9 (ab227037, Abcam, MA, USA), anti-CD4 (ab183685, Abcam, MA, USA), anti-Col8a1 (ab236653, Abcam, MA, USA) and anti-PDGFRa (MA541209, Thermo Scientific, MA) were used after de-paraffining. For the isolated fibroblast cell immunofluorescence staining, the cells were fixed in 4% PFA for 15 min after harvesting, and stained with anti-Col1a1 (NBP1-30054, Novus Biologicals, CO, USA), α-SMA (A5228; Sigma-Aldrich, MO, USA) overnight. Slides were then washed and incubated with conjugated secondary antibodies (Jackson ImmunoResearch Lab) Cy3 conjugated donkey anti-rat secondary antibody (1:300, Cat #: 712-165-153) and Alexa 647 conjugated donkey anti-rabbit secondary antibody (1:300, Cat #: 711-605-152) and Alexa 488 conjugated donkey anti-rabbit secondary antibody (1:300, Cat #:711-545-152) for 60 min at room temperature. Cell nuclei were stained with 4′,6-diamidino-2-phenylindole (DAPI, 422801, Biolegend, CA, USA). To distinguish true target staining from background, antibody specificity was evaluated using a polyclonal rabbit IgG isotype control instead of primary antibody. And for secondary antibody only controls, staining protocols were performed as described above without adding respective primary antibodies. Images were acquired on a Carl Zeiss LSM 880 confocal microscope using Zen black software version 2.3 SP1 (BIDMC confocal imaging and IHC core facility). The mean fluorescence intensity was measured using computer-assisted image quantification (ImageJ, NIH, Maryland, USA) and the CD4+IL9+ T cells and CD4+KLF10+ T cells were counted manually. Investigators were blinded to the sources of samples. Representative images were selected without bias and had characteristics typical of the data or overall trend.

### Mononuclear Cell Preparation and Flow Cytometry

Mononuclear cells for flow cytometry were isolated from the aorta and spleen to detect the characterization of different cell populations. Cells from spleens were isolated by grinding and filtering through 40 μm strainer. Single cells from aortic tissue were acquired after aorta perivascular adventitial tissue digestion. Mouse aortas were digested by using an optimized digestion enzyme mix recipe (Collagenase I 450 U/mL, Collagenase XI 125 U/mL, DNase I 60 U/mL, Hyaluronidase 60 U/mL, and Elastase 50 ng/ml). After that, samples were resuspended to obtain single cell suspensions.

After preparing the single cell suspension according to the above methods, the samples from murine spleen and aorta were sequentially filtered through 40 μm strainers. Following the manufacturer's instructions, appropriately fluorescently labeled antibodies were added at predetermined optimum concentrations and incubated on ice for 20 minutes in the dark for cell-surface staining. For analysis of cytokine production, aortic cells were stimulated in 48-well plates for 4-6 hours with phorbol 12-myristate 13-acetate (PMA, 16561-29-8, Sigma, MA, USA. 50 ng/mL), ionomycin (10634, Sigma Aldrich, MA, USA. 500 ng/mL), then treated with brefeldin A (10 μg/mL; Sigma Aldrich) 2 h later for additional 3 h. Cells were stained with LIVE/DEAD Cell Stain (Invitrogen), followed by staining for cell surface markers, and then fixed and permeabilized with the Cytofix/Cytoperm kit (554714BD, Biosciences) for intracellular staining. After washing with PBS, centrifuging at 350×g for 5 minutes, samples were resuspended for flow cytometric analysis (BD FACS Analyzer LSR, or BD FACS Analyzer Symphony). The antibodies for flow cytometry are attached in Table 12. All the flow data analysis were analyzed by FlowJo 10.7.1. Table 12 is shown below.

TABLE 12
Flow Cytometry antibodies list

| Name | Fluorochrome | Catalog | Trade name |
|---|---|---|---|
| anti-mouse CD45 | BUV563 | 612924 | BD |
| anti-mouse CD3 | Pacific Blue | 155612 | BioLegend |
| anti-mouse F480 | PE | 123109 | BioLegend |
| anti-mouse CD4 | PE-Cyanine7 | 100422 | BioLegend |
| anti-mouse CD8 | PerCP-Cy5.5 | 100734 | BioLegend |
| anti-mouse IFNΨ | FITC | 11-7311-82 | eBioscience |
| anti-mouse IL4 | PE | 12-7041-82 | eBioscience |
| anti-mouse IL9 | APC | 514106 | BioLegend |
| anti-mouse IL17 | FITC | 506908 | BioLegend |
| anti-mouse FoxP3 | ALEXA FLUOR ® 488 | 53-5773-82 | eBioscience |

### Cell Isolation and Cell Culture

CD3+, CD3− and CD4+ cells were isolated from mouse spleen. Splenic cells were isolated by grinding and filtering through 40 μm strainer. Following the manufacturer's instructions, CD4+ T cells were isolated by using the CD4+ T Cell Isolation Kit (130-104-454, Miltenyi Biotec, Germany). CD3+ T cells were isolated by using CD3ε MicroBead Kit, mouse (130-094-973, Miltenyi Biotec, Germany), and the rest of untouched cells were considered as splenic CD3− cells. All the T cells were cultured in Gibco RPMI Media 1640 with 10% FBS and 1% P/S and activated by using Mouse T-Activator CD3/CD28 for T-Cell expansion and activation (11452D, Gibco, MA, USA). CD45-CD90.2+ fibroblast cells were isolated from mouse descending aorta by using CD45-Microbeads (130-052-301, Miltenyi Biotec, Germany), PE-anti-CD90.2 antibody (553006, BD Biosciences, MA, USA) and anti-PE Microbeads (130-048-801, Miltenyi Biotec, Germany). Briefly, after preparing the aortic single cell suspension according to the above methods, the aortic cells were washed and filtered through a 70 μm filter with 2 ml 0.4% BSA-DPBS. The cell suspensions were then used for subsequent cell isolation. CD45− cells were first isolated by using CD45+ Microbeads. After centrifugation of CD45− cell suspensions at 300×g for 10 min and removal of supernatants, the non-CD45 cells were stained with PE anti-CD90.2, and then incubated with anti-PE Microbeads. Magnetically labeled CD45-CD90.2+ cells were collected and used as CD45-CD90.2+ fibroblasts. The purity of isolated fibroblasts in this method is about 97.2%, and fibroblasts were cultured in Gibco Dulbecco's Modified Eagle Medium (DMEM) supplemented with 10% FBS and 1% P/S.

HEK293T cells (CRL-3216, ATCC) were cultured in DMEM supplemented with 10% FBS, 1% L-glutamine (2 mM final), and 1% P/S.

### Cell Transfection

Transfection was performed using Lipofectamine 2000 (11668019, Invitrogen, MA, USA) as described in the manufacturer's protocol. A KLF10 expression vector in pcDNA3.1(+) plasmid was used.

### Luciferase Reporter Assay

The 119 promoter reporter was acquired from GeneCopoeia (MPRM39766-PG04; NM_008373). 5′ truncation deletions of the 119 promoters were generated from truncation of the WT 119 promoter vector relative to the transcriptional start site (TSS): mutant1 TSS −370; mutant2 TSS −329; and mutant3 TSS −252. HEK293T cells were co-transfected with 1 μg luciferase plasmid vector with 50 nM HDAC1 (s119558, Thermofisher, MA, USA), or non-specific siRNA (4390843, Thermofisher, MA, USA) for 72 hours. Analysis of luciferase activity with Secrete-Pair Dual Luminescence Assay (LF032, GeneCopoeia, MD, USA) and standard 96-well plate reader.

### Chromatin Immunoprecipitation (ChIP)

ChIP assay was performed according to the manufacturer's protocol from Upstate, using the ChIP assay kit (#9003, Cell signal) with modifications. Briefly, isolated mouse primary CD4+ T cells were treated with Ang II (200 nM, 12 h) and PBS (vehicle control). Cells were cross-linked with 1% formaldehyde for 15 min at room temperature, and then the reaction was stopped by incubating in glycine with a final concentration of 0.125 M for 5 min. Cells were washed three times with cold PBS and harvested by scraping with cell scraper. Then the cells were lysed in the SDS lysis buffer (1% SDS, 10 mM EDTA, and 50 mM Tris-HCl, pH 8.1) on ice for 10 min. The samples were sonicated into DNA fragments of 0.2-1 kb (checked by agarose gel electrophoresis/ethidium bromide staining) and microcentrifuged at maximal speed for 10 min at 4° C. The supernatant was precleared by rotating with 60 I of Salmon Sperm DNA/protein-agarose slurry for 30 min at 4° C. and then aliquoted after centrifugation. 20 μl was saved as input and 200 μl (equal to one-fifth the number of cells from one 100% confluent 15-cm dish) was used for each antibody. Each 200 μl supernatant was diluted with 800 μl of ChIP dilution buffer (0.01% SDS, 1.1% Triton X-100, 1.2 mM EDTA, 16.7 mM Tris-HCl, pH 8.1, and 167 mM NaCl) and incubated with the specific antibody (1 g/sample) at 4° C. overnight. A mock precipitation without antibody was used as negative control. The next day, 60 μl of salmon sperm DNA/protein-agarose slurry was added to each sample and incubated at 4° C. for another 2-4 h. The beads were then washed for 3-5 min with 1 ml of each buffers listed: low salt wash buffer (0.1% SDS, 1% Triton X-100, 2 mM EDTA, 20 mM Tris-HCl, pH 8.1, 150 mM NaCl), high salt wash buffer (0.1% SDS, 1% Triton X-100, 2 mM EDTA, 20 mM Tris-HCl, pH 8.1, 500 mM NaCl), and LiCl wash buffer (0.25 M LiCl, 1% IGEPAL-CA630, 1% deoxycholic acid (sodium salt), 1 mM EDTA, 10 mM Tris-HCl, pH 8.1). After all washes, pellets were suspended by vertex with 150 μl of freshly prepared elution buffer (0.1 M NaHCO₃, 1% SDS) for 15 min, and then supernatant was collected. This elution progress was repeated once again, and in total 300-1 elutes were collected. The one-tenth input was diluted with dilution buffer to a total volume of 300 μl. Elutes and diluted inputs were incubated in 0.3 M NaCl at 65° C. for 4 h to reverse formaldehyde cross-linking. Then 10 I of 0.5 M EDTA, 20 μl of 1 M Tris-HCl, pH 6.5, and 20 g of proteinase K were added to the sample and incubated at 45° C. for 1 h. DNA was extracted with phenol/chloroform and then incubated with 10 g of glycogen in 75% ethanol at 20° C. overnight. After precipitation by centrifuging at 12,000 g for 30 min at 4° C., the recovered DNA pellets were dissolved in 30 μl of distilled water. Amplifications were performed using RT qPCR with SYBR Green Master Mix (GoTag PCR system, Promega M7122). The qPCR primers used are attached in Table 13.

TABLE 13
Primers list

| Gene | Organism | Forward 5′-3′ | Reverse 5′-3′ |
|---|---|---|---|
| Klf10 | Mus | TTCTCTCCAGCAAGCTTCGGA | TCACTCTGCTCAGCTTTGTCCC |
| | musculus | (SEQ ID NO: 1) | (SEQ ID NO: 21) |
| Il9 | Mus | ATGTTGGTGACATACATCCTTGC | TGACGGTGGATCATCCTTCAG |
| | musculus | (SEQ ID NO: 2) | (SEQ ID NO: 22) |
| Control | Mus | ATGTTGGTGACATACATCCTTGC | TCTGGGGTAGCAGGCAAGTA |
| | musculus | (SEQ ID NO: 3) | (SEQ ID NO: 23) |
| Binding | Mus | TTCCCCTGATATCCCCAGTGT | CCAGGTCTGTGTTTTCCCCTC |
| site 1 | musculus | (SEQ ID NO: 4) | (SEQ ID NO: 24) |
| Binding | Mus | TGTGACCCCTTCATTACCACC | ATCTAGCCCCAACCCCCTTTA |
| site 2 | musculus | (SEQ ID NO: 5) | (SEQ ID NO: 25) |
| Binding | Mus | CTGGGCTTTCATGGAAAGTGT | TGCCAGGAAATACCATTCCTCA |
| site 3 | musculus | (SEQ ID NO: 6) | (SEQ ID NO: 26) |
| Binding | Mus | TTTCCTGGCATAAGACAGCCT | ACCGGGAAAAACCCTGACAT |
| site 4 | musculus | (SEQ ID NO: 7) | (SEQ ID NO: 27) |
| Acta2 | Mus | GTCCCAGACATCAGGGAGTAA | TCGGATACTTCAGCGTCAGGA |
| | musculus | (SEQ ID NO: 8) | (SEQ ID NO: 28) |
| Col1a1 | Mus | GCTCCTCTTAGGGGCCACT | CCACGTCTCACCATTGGGG |
| | musculus | (SEQ ID NO: 9) | (SEQ ID NO: 29) |
| Col3a1 | Mus | CTGTAACATGGAAACTGGGGAAA | CCATAGCTGAACTGAAAACCACC |
| | musculus | (SEQ ID NO: 10) | (SEQ ID NO: 30) |
| Col5a1 | Mus | CTTCGCCGCTACTCCTGTTC | CCCTGAGGGCAAATTGTGAAAA |
| | musculus | (SEQ ID NO: 11) | (SEQ ID NO: 31) |
| Col8a1 | Mus | ACTCTGTCAGACTCATTCAGGC | CAAAGGCATGTGAGGGACTTG |
| | musculus | (SEQ ID NO: 12) | (SEQ ID NO: 32) |
| Mmp9 | Mus | CTGGACAGCCAGACACTAAAG | CTCGCGGCAAGTCTTCAGAG |
| | musculus | (SEQ ID NO: 13) | (SEQ ID NO: 33) |
| Angptl1 | Mus | GGATGTGCTGTCTAGGCAGAA | TTCATGTTCCGGCTTTCCTTT |
| | musculus | (SEQ ID NO: 14) | (SEQ ID NO: 34) |
| Fmod | Mus | AGCAGTCCACCTACTACGACC | CAGTCGCATTCTTGGGGACA |
| | musculus | (SEQ ID NO: 15) | (SEQ ID NO: 35) |
| Il9r | Mus | TCCTGGTTCCTGATCTACAGC | TGTGTTTGATTTCAGTCACCTGG |
| | musculus | (SEQ ID NO: 16) | (SEQ ID NO: 36) |
| Agtr1a | Mus | AACAGCTTGGTGGTGATCGTC | CATAGCGGTATAGACAGCCCA |
| | musculus | (SEQ ID NO: 17) | (SEQ ID NO: 37) |
| Agtr1b | Mus | TGGCTTGGCTAGTTTGCCG | ACCCAGTCCAATGGGGAGT |
| | musculus | (SEQ ID NO: 18) | (SEQ ID NO: 38) |
| Agtr2 | Mus | AACTGGCACCAATGAGTCCG | CCAAAAGGAGTAAGTCAGCCAAG |
| | musculus | (SEQ ID NO: 19) | (SEQ ID NO: 39) |
| β-actin | Mus | GGCTGTATTCCCCTCCATCG | CCAGTTGGTAACAATGCCATGT |
| | musculus | (SEQ ID NO: 20) | (SEQ ID NO: 40) |

### Co-Immunoprecipitation (Co-IP)

Isolated splenic T cells were washed with PBS and homogenized in the Co-IP lysis buffer (20 mm Hepes, pH 7.4, 125 mm NaCl, 1% Triton X-100, 10 mm EGTA, 2 mm Na3VO4, 50 mm NaF, 20 mm ZnCl2, 10 mm sodium pyrophosphate, 1 mm dithiothreitol, and 1 mm phenylmethylsulfonyl fluoride). 1×complete protease inhibitor mixture (P8340-1 ML, Sigma, MA, USA) was added before use. After centrifugation (12,000×g in a microcentrifuge at 4° C. for 15 min) supernatant fractions were collected and incubated with antibodies and G/A protein magnetic beads (PIERCE™ Protein G Magnetic Beads 88847, Thermo Scientific, MA, USA) for 2 h at 4° C. on a rotary shaker. Corresponding isotype IgG was used as a negative control. The beads were washed three times, and the precipitated protein complexes were analyzed with Western blot.

### Calcium Flux Assay

Calcium flux assay was performed on isolated primary aortic fibroblasts. Calcium flux was determined by using the Fluo-8 No Wash Calcium Assay kit (ab112129, Abcam, MA, USA). In brief, fibroblasts were incubated with Fluo-8 in calcium-free Hanks' balanced salt solution (HHBS) at 37° C. for 30 min and subsequently incubated at room temperature for additional 30 min according to the in the manufacturer's protocol. Images for the calcium flux assay were acquired using the Zeiss LSM 880 laser-scanning confocal microscope's FAST mode for Airyscan module. Time-series images of fibroblasts were captured to observe dynamic intensity changes for a set field of view for a total time of 200 seconds. Images were captured every 0.5 seconds using the Plan-Apochromat 10×/0.45 NA objective lens. Immediately after adding Ang II (200 nM), recombinant mIL-9 (200 ng/mL), or Ang II (200 nM) plus recombinant mIL-9 (200 ng/mL), fluorescence intensity of fibroblasts was monitored. Time-series were then analyzed using ImageJ where total fluorescence intensity was calculated at every 5 seconds and plotted against time (seconds). Regions of Interest (ROIs) were placed to subtract intensity from any non-specific stained debris. Time-course changes in calcium levels were expressed as delta fluorescence ratio F/F0 or delta F/F0 relative to control (F is the fluorescence intensity at a given time, and F0 is the initial resting fluorescence intensity prior to stimuli). Background signal (signal from the area without cells) was subtracted from all data.

### RNA Isolation and Real-Time Quantitative PCR

Total RNA was extracted by using Trizol reagent following the manufacturer's protocol (15596-026 Invitrogen, MA, USA). The concentration and quality control of RNA was examined using NanoDrop 2000 (Thermo Fisher, MA, USA). cDNA was produced using High-Capacity cDNA Reverse Transcription Kit (4368814, Thermofisher, MA, USA). mRNAs expression levels were normalized to β-actin. A list of primers is presented in Table 13.

### Published Human Microarray Data and Mouse RNA-Seq Analysis

We reanalyzed the published human microarray data from peripheral blood mononuclear cells (PBMCs) of hypertensive patients with left ventricular remodeling (GSE74144) and PBMCs of controlled and uncontrolled hypertensives (GSE71994). Normalization and differential express analysis were performed by using Limma-Voom as described in Shakya et al. Adv. Exp. Med. Biol. 680:139-147 (2010). Genes with adjusted p-value <0.05 and log 2 fold-change (>0.1) were called as differentially expressed genes for each comparison. Differentially expressed upstream transcription factors were shown in volcano plot (ggplot2 package). Mouse RNA-Seq analysis of isolated splenic T cells from sham and angiotensin II treated WT mice (GSE143809) was re-analyzed using Limma-Voom. Differentially expressed upstream transcription factors were shown in volcano plot (ggplot2 package).

### Bulk RNA-Seq Analysis

RNA-Seq transcriptomic analysis was performed after ribodepletion and library construction by using Illumina performed after ribodepletion and standard library construction using Illumina HiSeq2500 V4 2×150 PE (Genewiz). All samples were processed by using a pipeline published in the bcbio-nextgen project (bcbionextgen.readthedocs.org/en/latest/). Raw reads were filtered and examined for quality control through running FastQC (bioinformatics.babraham.ac.uk/projects/fastqc/) and filtered reads were used to generate library and further analysis. Trimmed reads were aligned to UCSC build mm10 of the mouse genome and augmented with transcript information from Ensembl releases 86 (H. sapiens) using STAR. Total gene hit counts and CPM values were calculated for each gene and downstream differential expression analysis between specified groups was performed using DESeq2 and an adapted DESeq2 algorithm that excludes overlapping reads. Genes with adjusted p-value <0.05 and log 2 fold-change (>1.5) were called as differentially expressed genes for each comparison. The mean quality score of all samples was 35.91 with a range of 27,963,821-45,892,062 reads per sample. All samples had at least >90% of mapped fragments over total fragments. RNA-seq datasets will be deposited in a public repository upon publication.

### Pathway Enrichment Analysis

Differentially expressed genes (DEGs) were identified as being at least 1.5-fold change and adjusted p-value <0.05 (false discovery rate). The DEGs were visualized using hierarchical clustering plot. DEGs were subjected to gene set enrichment analyses by using Ingenuity Pathway Analysis (IPA) software and DAVID functional annotation tool. The pathway activity (Z score) was computed to determine whether the activity of canonical pathways is increased or decreased on the basis of differentially expressed genes in the data sets. The significant values for the canonical pathways were calculated by Fisher exact test. R package GOplot as described in Walter et al. Bioinformatics. 31:2912-2914 (2015) was used for visualization of pathway enrichment analysis on the set of DEGs (adjusted P-value <0.05).

### Single-Cell RNA Sequencing

Two aortas from TKO mice and two from Cre mice after Ang II treatment were collected and digested for single-cell sequencing. All portions of the scRNA-seq workflow (single-cell suspension preparation, library preparation, quality control PCR) were performed at the biopolymer facility in Harvard Medical School and sequencing was performed by Novogene, Inc. Freshly digested aortic cells suspension samples were diluted to target 10,000 cells for capture. The cells were processed using a 10×Genomics microfluidics chip to generate barcoded Gel Bead-In Emulsions according to manufacturer protocols. Indexed single-cell libraries were then created according to 10× Genomics specifications (Chromium Next GEM Single Cell 3′ v3.1-Dual Index Libraries). Samples were multiplexed and sequenced in pairs on an Illumina HiSeq 4000 device. Two aortas from TKO and two aortas from control mice were sequenced at high sequencing depth (163,200 reads per cell).

### Single-Cell RNA Sequencing Data Analysis

The sequenced data were processed into expression matrices with the Cell Ranger Single-cell software 6.0.0 (10× Genomics). FASTQ files were obtained from the base-call files from HiSeq4000 sequencer and subsequently aligned to the mouse transcriptome (mm10). Cellular barcodes and unique molecular identifiers (UMIs) were filtered and corrected by Cell Ranger pipeline. The filtered counts matrices were imported using the Seurat package (Seurat_4.1.0) in R (version 4.1.2) as described in Satija et al. Nat. Biotechnol. 33:495-502 (2015). Filtering during this step included only genes detected in >3 cells, cells with >400 distinct genes and >600 UMI. Cells with >20% mitochondrial percentage were excluded. A total of 53,346 sequenced cells combining all four samples met these quality control thresholds Data normalization and scaling were performed using the SCTransform command under default settings in Seurat. Samples were integrated following the default integration guidelines from satijalab.org/seurat/articles/integration_introduction.html. Principal component analysis and nonlinear dimensional reduction using Uniform Manifold Approximation and Projection (UMAP) were applied over the integrated dataset using 25 dimensions. A combination of previously known markers and newly identified markers using the FindAllMarkers function were used to assign cell types after cell clustering, using a resolution of 0.9, ensuring a separation of known major aortic cell types. A total of 10 major aortic cell types were identified. The FindAllMarkers function, applying Wilcoxon rank-sum test with default parameters, was used to obtain the significant markers for each cluster (Table 5).

The fibroblasts component was further subclustered, and data normalization, scaling were re-performed using the SCTransform command under default settings in Seurat. Principal component analysis and nonlinear dimensional reduction using UMAP were applied over the integrated dataset using 20 dimensions. A combination of previously known markers and newly identified markers using FindAllMarkers function were used to assign cell types after cell clustering, using a resolution of 0.5 (FIG. 18C), ensuring a separation of known major aortic cell types. A total of nine fibroblast subclusters were identified. The newly identified markers for each fibroblast subcluster were found using FindAllMarkers (Table 7). Gene ontology functional pathway analysis (DAVID) was performed on the significantly enriched markers (Bonferroni-adjusted p-value <0.05) in each FBS subcluster. For the differential abundance analysis, we followed the online instructions—(bioconductor.org/books/3.14/OSCA.multisample/differential-abundance.html #differential-abundance) from the Multi-Sample Single-Cell Analyses with Bioconductor section. RNA velocity analysis was performed using Velocyto (0.17.16) in python as described by La Manno et al. Velocity can estimate the RNA velocities of single cells by distinguishing unspliced and spliced mRNAs in standard single-cell RNA-sequencing data. Loom files were generated by Velocyto for each sample and concatenated into a Seurat object which were converted to h5ad format using hdf5r (1.3.5) and loomR (0.2.1.9) packages. To visualize velocity on the original UMAP embedding a new anndata was created by merging the velocity and original Seurat objects using the utils.merge ( ) function in scVelo (0.1.25). RNA Velocity in dynamic mode was performed using scanpy (1.7.1) and scVelo (0.2.3). Add module score analysis was performed using Seurat, which calculates the average expression levels of each cluster on s single-cell level, subtracted by the aggregated expression of control feature sets. K-means clustering (k value set to 3) analysis was performed using clustered Dot plot function in scCustomize R package (zenodo.org/record/5834562 #.Ygcphe7MJb8).

### Statistical Analysis

All data are presented as mean±standard error of the mean (SEM). Data are analyzed by GraphPad Prism V9.3.1 statistical software (GraphPad Software Inc, San Diego, CA). For experiments with small sample size (n<6), power calculations were not performed, and P-values were determined by non-parametric analysis. Other data were checked for normality before analysis by Shapiro-Wilk test. For normal distributed data, an unpaired two-tailed Student's t test was used for comparisons between two groups, one-way ANOVA with Tukey post hoc tests for comparisons between multiple groups, and two-way ANOVA for comparisons between multiple groups when there were 2 experimental factors. If the data are not normal or if n is too small to assess normality, nonparametric unpaired two-tailed Mann-Whitney U test was used for comparisons between two groups, and unpaired Mann-Whitney test with Bonferroni-Dunn's multiple comparisons tests for comparisons between multiple groups when there were 2 experimental factors. Wald Test in DESeq2 was used for bulk RNA sequence analysis and Wilcox test in Seurat was used for single-cell sequence analysis. P value of <0.05 was considered as statistical significance.

### OTHER EMBODIMENTS

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present invention that come within known or customary practice within the art to which the invention pertains and may be applied to the essential features hereinbefore set forth, and follows in the scope of the claims.

1. A method for treating a hypertensive disease or condition, a cardiovascular disease, or chronic kidney disease in a subject in need thereof, the method comprising administering an effective amount of an anti-interleukin-9 (IL-9) antibody, or an antigen-binding fragment thereof, to the subject, thereby treating the hypertensive disease or condition, the cardiovascular disease, or the chronic kidney disease.

2. A method for reducing fibrosis in a subject in need thereof, the method comprising administering an effective amount of an anti-IL-9 antibody, or an antigen-binding fragment thereof, to the subject, thereby reducing fibrosis in the subject.

3. The method of claim 2, wherein the fibrosis comprises perivascular fibrosis.

4. The method of claim 3, wherein the perivascular fibrosis occurs in the subject's aorta, heart, and/or kidney.

5. The method of claim 2, wherein the subject has a hypertensive disease or condition, a cardiovascular disease, or a chronic kidney disease.

6. The method of claim 1, wherein the hypertensive disease or condition is a heart disease or a kidney disease.

7. The method of claim 1, wherein:
(i) the hypertensive disease or condition comprises hypertension; hypertensive heart disease; heart failure with preserved ejection fraction; coronary heart disease; hypertensive-associated end organ damage; or any combination thereof;
(ii) the cardiovascular disease comprises coronary artery disease, atherosclerosis, myocardial infarction, heart failure, atrial fibrillation, cerebrovascular disease, stroke, peripheral artery disease, aortic aneurysm, retinopathy, or any combination thereof; or
(iii) the chronic kidney disease comprises end stage renal disease (ESRD).

8. The method of claim 7, wherein the hypertension comprises isolated systolic, malignant, or resistant hypertension.

9. The method of any one of claims 1-8, wherein the method results in: (i) a decreased expression level of Alox15 and/or Haptoglobin (Hp) in a sample obtained from the subject relative to a reference expression level of Alox15 and/or Hp; and/or (ii) a decreased expression level of one or more fibrotic genes in a sample obtained from the subject relative to a reference expression level of the one or more fibrotic genes.

10. The method of claim 9, wherein the one or more fibrotic genes comprises Alox15, Col8a1, Mmp2, Fmod, and/or Angptl1.

11. The method of any one of claims 1-8, wherein the method results in an improvement in heart function, kidney function, or vascular remodeling compared to a subject who has not been treated with the anti-IL-9 antibody or the antigen-binding fragment thereof.

12. The method of any one of claims 1-8, wherein the method results in:
(i) a decreased fibroblast intracellular calcium mobilization;
(ii) a decreased fibroblast activation or differentiation;
(iii) a reduced production of one or more extracellular matrix (ECM) components;
(iv) an improved left ventricular global longitudinal strain (LV GLS);
(v) a decreased pulse wave velocity (PWV);
(vi) an increased circumferential (Circ) strain;
(vii) a decreased ratio of albumin to creatinine;
(viii) a decreased kidney injury molecule-1 (KIM-1) expression level;
(ix) a decreased calcium deposition in the perivascular adventitia; or
(x) any combination of (i) through (ix),
compared to a subject who has not been treated with the anti-IL-9 antibody or the antigen-binding fragment thereof.

13. The method of claim 12, wherein the one or more ECM components comprises collagen.

14. The method of any one of claims 1-8, wherein the anti-IL-9 antibody is an anti-human IL-9 antibody.

15. The method of claim 14, wherein the anti-IL-9 antibody comprises enokizumab.

16. The method of any one of claims 1-8, wherein the anti-IL-9 antibody, or the antigen-binding fragment thereof, is administered to the subject as a monotherapy.

17. The method of any one of claims 1-8, wherein the anti-IL-9 antibody, or the antigen-binding fragment thereof, is administered to the subject in combination with one or more additional therapeutic agents.

18. The method of claim 17, wherein the one or more additional therapeutic agents comprise an antihypertensive agent, an anti-arrhythmic agent, an anticoagulant agent, an anti-platelet agent, a cholesterol-lowering agent, digoxin, a nitrate, or any combination thereof.

19. The method of claim 18, wherein the anti-hypertensive agent comprises an angiotensin II receptor antagonist, an angiotensin-converting enzyme (ACE) inhibitor, a diuretic, a calcium channel antagonist, an adrenergic receptor antagonist, a vasodilator, a renin inhibitor, an aldosterone receptor antagonist, an alpha-2 adrenergic receptor agonist, an endothelin receptor blocker, or any combination thereof.

20. The method of any one of claims 1-8, wherein the subject is a human.

21. A kit comprising an anti-IL-9 antibody, or an antigen-binding fragment thereof, and a package insert comprising instructions to administer the anti-IL-9 antibody, or the antigen-binding fragment thereof, to a subject to (i) treat a hypertensive disease or condition, a cardiovascular disease, or chronic kidney disease in a subject in need thereof, and/or (ii) reduce fibrosis (e.g., perivascular fibrosis) in a subject in need thereof.

22. An anti-IL-9 antibody, or an antigen-binding fragment thereof, for use in (i) treating a hypertensive disease or condition, a cardiovascular disease, or chronic kidney disease in a subject in need thereof, and/or (ii) reducing fibrosis (e.g., perivascular fibrosis) in a subject in need thereof.

23. A method for treating a hypertensive disease or condition, a cardiovascular disease, or chronic kidney disease in a subject in need thereof, the method comprising administering an effective amount of a Kruppel-like factor 10 (KLF10) agonist to the subject, thereby treating the hypertensive disease or condition, the cardiovascular disease, or the chronic kidney disease.

24. A method for reducing fibrosis in a subject in need thereof, the method comprising administering an effective amount of a KLF10 agonist to the subject, thereby reducing fibrosis in the subject.

25. The method of claim 24, wherein the fibrosis comprises perivascular fibrosis.

26. The method of claim 25, wherein the perivascular fibrosis occurs in the subject's aorta, heart, and/or kidney.

27. The method of claim 24, wherein the subject has a hypertensive disease or condition, a cardiovascular disease, or a chronic kidney disease.

28. The method of claim 23, wherein the hypertensive disease or condition is a heart disease or a kidney disease.

29. The method of claim 23, wherein:
(i) the hypertensive disease or condition comprises hypertension; hypertensive heart disease; heart failure with preserved ejection fraction; coronary heart disease; hypertensive-associated end organ damage; or any combination thereof;
(ii) the cardiovascular disease comprises coronary artery disease, atherosclerosis, myocardial infarction, heart failure, atrial fibrillation, cerebrovascular disease, stroke, peripheral artery disease, aortic aneurysm, retinopathy, or any combination thereof; or
(iii) the chronic kidney disease comprises ESRD.

30. The method of claim 29, wherein the hypertension comprises isolated systolic, malignant, or resistant hypertension.

31. The method of any one of claims 23-30, wherein the KLF10 agonist comprises a small molecule agonist, recombinant KLF10, or a viral vector (e.g., adeno-associated viral vector) comprising a nucleic acid encoding KLF10.

32. The method of any one of claims 23-30, wherein the KLF10 agonist is administered to the subject as a monotherapy.

33. The method of any one of claims 23-30, wherein the KLF10 agonist is administered to the subject in combination with one or more additional therapeutic agents.

34. The method of claim 33, wherein the one or more additional therapeutic agents comprise an antihypertensive agent, an anti-arrhythmic agent, an anticoagulant agent, an anti-platelet agent, a cholesterol-lowering agent, digoxin, a nitrate, or any combination thereof.

35. The method of claim 34, wherein the anti-hypertensive agent comprises an angiotensin II receptor antagonist, an ACE inhibitor, a diuretic, a calcium channel antagonist, an adrenergic receptor antagonist, a vasodilator, a renin inhibitor, an aldosterone receptor antagonist, an alpha-2 adrenergic receptor agonist, an endothelin receptor blocker, or any combination thereof,

36. The method of any one of claims 23-30, wherein the subject is a human.

37. A kit comprising a KLF10 agonist and a package insert comprising instructions to administer the KLF10 agonist to a subject to (i) treat a hypertensive disease or condition, a cardiovascular disease, or chronic kidney disease in a subject in need thereof, and/or (ii) reduce fibrosis (e.g., perivascular fibrosis) in a subject in need thereof.

38. A KLF10 agonist for use in (i) treating a hypertensive disease or condition, a cardiovascular disease, or chronic kidney disease in a subject in need thereof, and/or (ii) reducing fibrosis (e.g., perivascular fibrosis) in a subject in need thereof.


SEQUENCE LISTING (54 sequences)

SEQ ID NO: 1 - DNA, 21 bp, synthetic construct
  source 1..21: mol_type = other DNA
ttctctccagcaagcttcgga

SEQ ID NO: 2 - DNA, 23 bp, synthetic construct
  source 1..23: mol_type = other DNA
atgttggtgacatacatccttgc

SEQ ID NO: 3 - DNA, 23 bp, synthetic construct
  source 1..23: mol_type = other DNA
atgttggtgacatacatccttgc

SEQ ID NO: 4 - DNA, 21 bp, synthetic construct
  source 1..21: mol_type = other DNA
ttcccctgatatccccagtgt

SEQ ID NO: 5 - DNA, 21 bp, synthetic construct
  source 1..21: mol_type = other DNA
tgtgaccccttcattaccacc

SEQ ID NO: 6 - DNA, 21 bp, synthetic construct
  source 1..21: mol_type = other DNA
ctgggctttcatggaaagtgt

SEQ ID NO: 7 - DNA, 21 bp, synthetic construct
  source 1..21: mol_type = other DNA
tttcctggcataagacagcct

SEQ ID NO: 8 - DNA, 21 bp, synthetic construct
  source 1..21: mol_type = other DNA
gtcccagacatcagggagtaa

SEQ ID NO: 9 - DNA, 19 bp, synthetic construct
  source 1..19: mol_type = other DNA
gctcctcttaggggccact

SEQ ID NO: 10 - DNA, 23 bp, synthetic construct
  source 1..23: mol_type = other DNA
ctgtaacatggaaactggggaaa

SEQ ID NO: 11 - DNA, 20 bp, synthetic construct
  source 1..20: mol_type = other DNA
cttcgccgctactcctgttc

SEQ ID NO: 12 - DNA, 22 bp, synthetic construct
  source 1..22: mol_type = other DNA
actctgtcagactcattcaggc

SEQ ID NO: 13 - DNA, 21 bp, synthetic construct
  source 1..21: mol_type = other DNA
ctggacagccagacactaaag

SEQ ID NO: 14 - DNA, 21 bp, synthetic construct
  source 1..21: mol_type = other DNA
ggatgtgctgtctaggcagaa

SEQ ID NO: 15 - DNA, 21 bp, synthetic construct
  source 1..21: mol_type = other DNA
agcagtccacctactacgacc

SEQ ID NO: 16 - DNA, 21 bp, synthetic construct
  source 1..21: mol_type = other DNA
tcctggttcctgatctacagc

SEQ ID NO: 17 - DNA, 21 bp, synthetic construct
  source 1..21: mol_type = other DNA
aacagcttggtggtgatcgtc

SEQ ID NO: 18 - DNA, 19 bp, synthetic construct
  source 1..19: mol_type = other DNA
tggcttggctagtttgccg

SEQ ID NO: 19 - DNA, 20 bp, synthetic construct
  source 1..20: mol_type = other DNA
aactggcaccaatgagtccg

SEQ ID NO: 20 - DNA, 20 bp, synthetic construct
  source 1..20: mol_type = other DNA
ggctgtattcccctccatcg

SEQ ID NO: 21 - DNA, 22 bp, synthetic construct
  source 1..22: mol_type = other DNA
tcactctgctcagctttgtccc

SEQ ID NO: 22 - DNA, 21 bp, synthetic construct
  source 1..21: mol_type = other DNA
tgacggtggatcatccttcag

SEQ ID NO: 23 - DNA, 20 bp, synthetic construct
  source 1..20: mol_type = other DNA
tctggggtagcaggcaagta

SEQ ID NO: 24 - DNA, 21 bp, synthetic construct
  source 1..21: mol_type = other DNA
ccaggtctgtgttttcccctc

SEQ ID NO: 25 - DNA, 21 bp, synthetic construct
  source 1..21: mol_type = other DNA
atctagccccaacccccttta

SEQ ID NO: 26 - DNA, 22 bp, synthetic construct
  source 1..22: mol_type = other DNA
tgccaggaaataccattcctca

SEQ ID NO: 27 - DNA, 20 bp, synthetic construct
  source 1..20: mol_type = other DNA
accgggaaaaaccctgacat

SEQ ID NO: 28 - DNA, 21 bp, synthetic construct
  source 1..21: mol_type = other DNA
tcggatacttcagcgtcagga

SEQ ID NO: 29 - DNA, 19 bp, synthetic construct
  source 1..19: mol_type = other DNA
ccacgtctcaccattgggg

SEQ ID NO: 30 - DNA, 23 bp, synthetic construct
  source 1..23: mol_type = other DNA
ccatagctgaactgaaaaccacc

SEQ ID NO: 31 - DNA, 22 bp, synthetic construct
  source 1..22: mol_type = other DNA
ccctgagggcaaattgtgaaaa

SEQ ID NO: 32 - DNA, 21 bp, synthetic construct
  source 1..21: mol_type = other DNA
caaaggcatgtgagggacttg

SEQ ID NO: 33 - DNA, 20 bp, synthetic construct
  source 1..20: mol_type = other DNA
ctcgcggcaagtcttcagag

SEQ ID NO: 34 - DNA, 21 bp, synthetic construct
  source 1..21: mol_type = other DNA
ttcatgttccggctttccttt

SEQ ID NO: 35 - DNA, 20 bp, synthetic construct
  source 1..20: mol_type = other DNA
cagtcgcattcttggggaca

SEQ ID NO: 36 - DNA, 23 bp, synthetic construct
  source 1..23: mol_type = other DNA
tgtgtttgatttcagtcacctgg

SEQ ID NO: 37 - DNA, 21 bp, synthetic construct
  source 1..21: mol_type = other DNA
catagcggtatagacagccca

SEQ ID NO: 38 - DNA, 19 bp, synthetic construct
  source 1..19: mol_type = other DNA
acccagtccaatggggagt

SEQ ID NO: 39 - DNA, 23 bp, synthetic construct
  source 1..23: mol_type = other DNA
ccaaaaggagtaagtcagccaag

SEQ ID NO: 40 - DNA, 22 bp, synthetic construct
  source 1..22: mol_type = other DNA
ccagttggtaacaatgccatgt

SEQ ID NO: 41 - DNA, 3596 bp, Homo sapiens
  source 1..3596: mol_type = genomic DNA
aagcgagctccagtccgctgtcaagatgcttctggccatggtccttacctctgccctgctcctgtgctccgtggcaggccaggggtgtccaaccttggcggggatcctggacatcaacttcctcatcaacaagatgcaggtaggctgcagggggagcccatgggaaagacagctactgacaaagtgaaatatgtatgaggatgaaaaaactcggggctgactaaaggttcttatctctctatctactttaggaagatccagcttccaagtgccactgcagtgctaatgtgagtgaatgctctttaagaactttccaaattaattttaattttcacatctggaatcttcactctgaaatttcccttgcaggtgaccagttgtctctgtttgggcattccctctgtaagtatagtgaaataacataatgttgaccttggatttttttggtttgtttttaagtaaaaataagttgctttatttaatatttaatgttatacattgttgcttaatttaattgttacagattagtattccctgttaaaaccacattgttacaaattattcccttttaaaactacgatcttgaaatcctatattatgaacatttctttgtatttaattaactttatgcctcttgagaagtttgaacacttttcaacattaaaaaaagaatcctgaatatctttttagataggtggccatgtgcacaattaaataaaactggaactaaggatataataattgctgtagctcatatcatattgctttctaactcatttactgataactctagagttgtgaaacaatgtaaataaaatgacaactccttatctttcatctgtcatgaatgatctatgcgctatacctccccctccctgcctcctcccttcctccccaccaccctgttgtctgtctagctgattagagtgactgttggtttgaatgctgccctctgggcaggtagaggatctgaggttgtgagtggaaggagggcttccagagggccactgcccactacggcaggaaggatgggtggcaggaaagttctgattcctaattcaaactcctggttagggtgaggaggaggcacttctccaaggtgcagtgctttattctttctcatgcaaggcctgggagaatctgaagaatctgagcttcttgccctggctagggtaagacatcgcacccatcgcggtccatccattagatgagaagaggatagagtgccttctgggcaggaaccaggcagacagcacagcccctgtcccttggagtaccgtccatgtttttagctgctgctgaaataccagctgcattcaattgtcacatcccattagctggtgtgaaaaggcttttcctcactctgcactttcagacttacaagccttgaagccgggaagcacccgttgaaaagaacattcagagccgactatttcagggcccagagccctcatgtttcctggatgtaacatacaggaagtctcctccaggggatgtcactgtggaaaaatggcatcccctttaaatacgggagatcacttcctacattggcaagggacctgtctaaaaataatgcaagtttgagtaatggtgattaaataaaaatcatctctattatattgctctttgtgatatatttccaaagctgtcctcagaatatttctttgaataaatccttactatttaccaggacaactgcaccagaccatgcttcagtgagagactgtctcagatgaccaataccaccatgcaaacaagatacccactgattttcagtcgggtgaaaaaatcagttgaagtactaaagaacaacaagtgtccagtaagtttgttttcatatgtgatatgttcctgttggtgatttctatgtgaatggtgatgccaaccctgtttgaacacaaaaggatgataaagttggaattggtagttcaaggttgataaaagacatctaagaattttaatcagaagtaatataattaaagtgagatccactgaaacaatagaattaaagtgagatagatcattgttcctgacgaggccatttacttctctctactatggaataatgaaagaatcctttctgagtgtaattagaagctacaatctagagaatcagggatgtagctcacataatactaaattatcctagagattcaatgtactaactgaatggatgttgttaacagggatttttttttcctgttggttaaggaggttttgttttgttttggagacagagtcttgctctgttgcccaggctggagtgcagtggtgccatctgagctcactgcagcctctgcctcccgggttcaagtgattatcctgcctcagcctcccgagtagctggcattacaggtgcgtgccaccatgcctggctaatttttgtatttttaatagagatggggtttcaccatgttggccaggttgctctccaactcctgaactcaagtgatttgcccgccttgacctcccaaagtgctgggatgacaggtgtgagccaccatgcctggcctgcattaaggaggtatttaaagggcaatgcacccaggtcaaggtggaagcttgctactcatcctgaatgcccatccacacattcttttcttcagcatataccctagtccctgacagcagactgggatggcaagttgggtagaggtgacctccctctgttttttgggtattagcatctccacacaagatcctagaaggctgaaagccctgagctcagctgtttagctgcatgcgtttctaccatcaatggcatctagttctaagtgcttaatatatgctgtctcactgaataaatacataccttagggacaattattcaatttattactctcagtgaggttaactaatttgcctaaggctgcatatttgataagtggcagagctgagatttgaactcaggcctatatgacctcagagccccactcttagccattgtactgtcaaatgaccttggaaagacaacctaaaaggataatgatacaattttaggcctcaaagagtccccagaaaaggctttctctaatgcagagatttagggccacttaataggggtgtgtgtgtgtgtgtgtgtgtgtgtgtgtgtgtgtgtgtgtgtgtaaagacccctgaaatccaatttgaggtcaaccacctatgctgtctttacaccacatgagctagcctggacctgcccacctatttgctctgtgtctcaagccacttcccttcccatccccacaatcctcaccaccgactctggctcttggcaggtaggcttctggggctgcttggctctacatcatttgagtcactctgtccttatcaactttcatccccacagtatttttcctgtgaacagccatgcaaccaaaccacggcaggcaacgcgctgacatttctgaagagtcttctggaaattttccagaaagaaaagatgagagggatgagaggcaagatatgaagatgaaatattatttatcctatttattaaatttaaaaagctttctctttaagttgctacaatttaaaaatcaagtaagctactctaaatcagtatcagttgtgattatttgtttaacattgtatgtctttattttgaaataaat

SEQ ID NO: 42 - DNA, 454 bp, Homo sapiens
  source 1..454: mol_type = genomic DNA
ccgctgtcaagatgcttctggccatggtccttacctctgccctgctcctgtgctccgtggcaggccaggggtgtccaaccttggcggggatcctggacatcaacttcctcatcaacaagatgcaggaagatccagcttccaagtgccactgcagtgctaatgtgaccagttgtctctgtttgggcattccctctgacaactgcaccagaccatgcttcagtgagagactgtctcagatgaccaataccaccatgcaaacaagatacccactgattttcagtcgggtgaaaaaatcagttgaagtactaaagaacaacaagtgtccatatttttcctgtgaacagccatgcaaccaaaccacggcaggcaacgcgctgacatttctgaagagtcttctggaaattttccagaaagaaaagatgagagggatgagaggcaagatatgaagatgaaa

SEQ ID NO: 43 - protein, 144 aa, Homo sapiens
MLLAMVLTSALLLCSVAGQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKI

SEQ ID NO: 44 - DNA, 6942 bp, Homo sapiens
  source 1..6942: mol_type = genomic DNA
gcagacggcgctgagcgcggcggcggcgggagcggcgtcgagtgtctccgtgcgcccgtctgtggccaagcagccagcagcctagcagccagtcagcttgccgccggcggccaagcagccaaccatgctcaacttcggtgcctctctccagcagactgcggtaagtcatttggggatgcccctgtgcttcctcgcctggtcttgtctggggggccaaagggggcgcgaaccccgagccccggacatcagccatgcctgagaattggggctgcagcggagtcgtggggaaggaaagggcttcctgcctgcagactatgggcattagtgagggcgtgtgtgttcgggagggggtcgaaccagggggctgggatcttcagacagggacaggggtcttgctctagatgtactgaggggaagggacaactccgcatggagacccgagagggctggtgaggaggaggatgacgagcgggggaggagtggggagggggccgttgccctgcagcgaggttggggtacagtgtggaacgagagtcgcgggagggacaagggggctaccctcacctggtaccgaggagagatgcccggtctggctcaggaacgcgagccaggggaggaaggggctgctcgcaagcactcgggaggcggggtgtccacaggagaggcgagtgggctacccatcctgggggtctgaacccacgcttggaagacgggggctgctcagcagatagctcggggctgggggaaggggcgcctgtcgtcggagcggggggatgacacgagggcccgccgacagccctgggggacgagttgctcccccagacgtctggagggggactcttaccccaggggcaggtggccgccttctgacgactccttggaagaggcggggagccgcctctggggcaggagggtgggaagcgggacccagcagctggggcgggaaggtgggcggggggagctgggccgcgagacgcgggggccgcgcgtctggagccggggcctgtgggggctgcacgtctgggaacccgtggagacgcgcggcgggaggggcctggagctgccggaagccacggggagggggcgccgcgtctacagggagagacggggtggtctgaggtcccgtgggccgcgtgggcgggaagtgccgagggctcggccgggttcccgggggctgccggcgggaggagggcggcggccgggtttccgcggggacctggctcctcctcccgcatctgaggcggaggccgggggtgggccgggagcgggtgcgcccggggagcttctcgtccccgagtgacccagccccggcctccgccaggggcgggagcgaggcccggccccgctgtcggttcctaagggaacggcggctcgcggggagctggaaggggcgggcgcctcccggagcctggcgcccgcagccccgcccctccccgcgggcccggcgcgcacggccgggcggttggcggcggcgcccgcggcccctcccccgggtcgggcgcgggcgggggcccgcaagctcaggaagtaggggaaaggtgacggcgcggcaattcccagttcacgtttcctgcgcctcccggagcagccgctgatcacgctttgttcacatgcctcgccccctcctcgcccacgcccccgccccgggccgcacagccaatccgggctcgggccgccgagccggccggccaatggcgggggcaggggcgcggggacgtgcgagcggcgaggaatgttcccagtgactcacccgtgagcctcattgaggttagggcggccccatccgtcggtctccgccagctaggatctcccccgccgtccgcccccgcccctcgtctgcctcccccgcccgcgcctccgcccgcgcttccctttcctgcctcgcgccccactccctttcctcccctccctgtttcccttcctgtccttcccagctcacgctctctttccctgccgcctgcctttcttttttcctttttttgcattggcgtcttggggctgttacacacacgcgcgctgtccattgcagcttacataaaggcgggcgcgattatgcaattatattgttagcgatatttcaagagcaatggctcgttttcttaggatttcaacacgaaggcatcatgcatttttgaaaaactagtattgagaataataccttgcaacgtaaagaatgttttttggtatttttacacaatctctactttgaccaaacgagtctggacagttttcttttaatggaaaataggagaaatggtgagtagtaccatttttttttagcgtagttcaaagaactttactataaaacattaggtagttctacaaataagtgcaaaacctgtgctgttattttgtcatcaagcagttctttaaggaattaaaatagattctactttgattatgctgactttttaaagtcggtttttgcaaattccaagtagaatattaaattttttgattatccagtttttagtaatattgcattagctatgttgtagttatgtatggcaaaccattgtgtcaaatagattcagtaaaagggagcacaaatatgaagaggttgaaagttaattaaaataaacattagaatttttttctatgtatatcacttgatttttctttaccataatcaagtgatcaaattttagatcttaaataatgcaattttaacttgaaagataccctgaggatcccttatcttttgatttttatcccaaagtgcaaagtgactttccaaaggtcagacccataacaacaggccagagctctactacattttgtctttattagaaggttttggctattttttttttctatcagcaatttgtacctcaaaagacctcataccatacctgtaatatttaagaagataaattttttgccgcactcttagtttttttgcccgactccagttgcaggtagctactctggaaagtctatataaaatactccttttcttcttagccaaatacttctccttttggatcacaaatgcattgataattttcgtcttagtccccttaatggtagtaggtgtgcctctctcccatgaacggatatcgctttatcagtgttaaagtctaaatgttaacagaaaaaatgaggaagattttattgccactactgtgaggtttgggttacctacctaacaagtgtagctgaacttccttagtatcatagtaagtgtgagaggaaaattatttctgattttaatttggaaagttgtgtgagttgtaaaaaaaaaaaaaaaaaaaatttgctcatttttctgtgtgtcatttggatgacaaaaaatatgcttataagctttcctcttttgtttttataggaggaaagaatggaaatgatttctgaaaggccaaaagagagtatgtattcctggaacaaaactgcagagaaaagtgattttgaagctgtagaagcacttatgtcaatgagctgcagttggaagtctgattttaagaaatacgttgaaaacagacctgttacaccagtatctgatttgtcagaggaagagaatctgcttccgggaacacctgattttcatacaatcccagcatttgtaagtattattgtttttaagatagacgtaaattatataatttcttaaaatttatataataaactgtagatttctcatgctatgtatttcccttttctttagtgtttgactccaccttacagtccttctgactttgaaccctctcaagtgtcaaatctgatggcaccagcgccatctactgtacacttcaagtcactctcagatactgccaaacctcacattgccgcacctttcaaagaggaagaaaagagcccagtatctgcccccaaactccccaaagctcaggcaacaagtgtgattcgtcatacagctgatgcccagctatgtaaccaccagacctgcccaatgaaagcagccagcatcctcaactatcagaacaattcttttagaagaagaacccacctaaatgttgaggctgcaagaaagaacataccatgtgccgctgtgtcaccaaacagatccaaatgtgagagaaacacagtggcagatgttgatgagaaagcaagtgctgcactttatgacttttctgtgccttcctcagagacggtcatctgcaggtctcagccagcccctgtgtccccacaacagaagtcagtgttggtctctccacctgcagtatctgcagggggagtgccacctatgccggtcatctgccagatggttccccttcctgccaacaaccctgttgtgacaacagtcgttcccagcactcctcccagccagccaccagccgtttgcccccctgttgtgttcatgggcacacaagtccccaaaggcgctgtcatgtttgtggtaccccagcccgttgtgcagagttcaaagcctccggtggtgagcccgaatggcaccagactctctcccattgcccctgctcctgggttttccccttcagcagcaaaagtcactcctcagattgattcatcaaggataaggagtcacatctgtagccacccaggatgtggcaagacatactttaaaagttcccatctgaaggcccacacgaggacgcacacaggtaccagccacttcttatatctttagtgtttaaatgaagagattttggctgtgatcacacatttaaacccaggatgataaggaataacttgcctttagctagtttagaggatggtatttaccagtgcaaggtcctccaaaaggctgtattggaggccgctcacgagtgccataatccttgcactttaggagaccaaggtgggaggattgtttgagcataggagttcaaaaccagcctgtgcaacatactgagacctcatctctacaaataatttaaaaataagtatgtaaatactttattgatgtgcttttgtttcagtctctcaaatgatgattctgtagatgtctgagtagttactttaataaggtatctttaaagcctttgaatcatagttaaagtcaggaagtcttttcatttttggctttttttaagggatgaaaaaaaactgctctgttgcccaaactggtctcaaacttctgggctcaagctatcctcctgccccagccttctgagtagttgggactacaggtgcctactactccacccggctgccttatttcatgtggggaagagttttgtaaatgtgatcaagtgacaagttgaaattggagcaatacatatctcatattcatttgacagtttgatttattagcttttattatttaaaatgtatcttctcatatacttttagacagaattgaacatataatttacggcaatcttatgcataataatgatttgtatgtctttgttttaggagaaaagcctttcagctgtagctggaaaggttgtgaaaggaggtttgcccgttctgatgaactgtccagacacaggcgaacccacacgggtgagaagaaatttgcgtgccccatgtgtgaccggcggttcatgaggagtgaccatttgaccaagcatgcccggcgccatctatcagccaagaagctaccaaactggcagatggaagtgagcaagctaaatgacattgctctacctccaacccctgctcccacacagtgacagaccggaaagtgaagagtcagaactaactttggtctcagcgggagccagtggtgatgtaaaaatgcttccactgcaagtctgtggccccacaacgtgggcttaaagcagaagccccacagcctggcacgaaggccccgcctgggttaggtgactaaaagggcttcggccacaggcaggtcacagaaaggcaggtttcatttcttatcacataagagagatgagaaagcttttattcctttgaatattttttgaaggtttcagatgaggtcaacacaggtagcacagattttgaatctgtgtgcatatttgttactttacttttgctgtttatacttgagaccaacttttcaatgtgattcttctaaagcactggtttcaagaatatggaggctggaaggaaataaacattacggtacagacatggagatgtaaaatgagtttgtattattacaaatattgtcatctttttctagagttatcttctttattattcctagtctttccagtcaacatcgtggatgtagtgattaaatatatctagaactatcatttttacactattgtgaatatttggaattgaacgactgtatattgctaagagggcccaaagaattggaatcctccttaatttaattgctttgaagcatagctacaatttgtttttgcatttttgttttgaaagtttaacaaatgactgtatctaggcatttcattatgctttgaactttagtttgcctgcagtttcttgtgtagatttgaaaattgtataccaatgtgttttctgtagactctaagatacactgcactttgtttagaaaaaaaactgaagatgaaatatatattgtaaagaagggatattaagaatcttagataacttcttgaaaaagatggcttatgtcatcagtaaagtacctttatgttatgaggatataatgtgtgctttattgaattagaaaattagtgaccattattcacaggtggacaaatgttgatgttgtcctgttaatttataggcgttttttggggatgtggaggtagttgggtagaaaaattattagaacattcacttttgttaacagtatttctcttttattctgttatatagtggatgatatacacagtggcaaaacaaaagtacattgcttaaaatatatagtgaaaaatgtcactatatcttcccatttaacattgtttttgtatattgggtgtagatttctgacatcaaaacttggacccttggaaaacaaaagttttaattaaaaaaaatccttgtgacttacaatttgcacaatatttcttttgttgtactttatatcttgtttacaataaagaattccctttggta

SEQ ID NO: 45 - DNA, 2950 bp, Homo sapiens
  source 1..2950: mol_type = genomic DNA
agacggcgctgagcgcggcggcggcgggagcggcgtcgagtgtctccgtgcgcccgtctgtggccaagcagccagcagcctagcagccagtcagcttgccgccggcggccaagcagccaaccatgctcaacttcggtgcctctctccagcagactgcggaggaaagaatggaaatgatttctgaaaggccaaaagagagtatgtattcctggaacaaaactgcagagaaaagtgattttgaagctgtagaagcacttatgtcaatgagctgcagttggaagtctgattttaagaaatacgttgaaaacagacctgttacaccagtatctgatttgtcagaggaagagaatctgcttccgggaacacctgattttcatacaatcccagcattttgtttgactccaccttacagtccttctgactttgaaccctctcaagtgtcaaatctgatggcaccagcgccatctactgtacacttcaagtcactctcagatactgccaaacctcacattgccgcacctttcaaagaggaagaaaagagcccagtatctgcccccaaactccccaaagctcaggcaacaagtgtgattcgtcatacagctgatgcccagctatgtaaccaccagacctgcccaatgaaagcagccagcatcctcaactatcagaacaattcttttagaagaagaacccacctaaatgttgaggctgcaagaaagaacataccatgtgccgctgtgtcaccaaacagatccaaatgtgagagaaacacagtggcagatgttgatgagaaagcaagtgctgcactttatgacttttctgtgccttcctcagagacggtcatctgcaggtctcagccagcccctgtgtccccacaacagaagtcagtgttggtctctccacctgcagtatctgcagggggagtgccacctatgccggtcatctgccagatggttccccttcctgccaacaaccctgttgtgacaacagtcgttcccagcactcctcccagccagccaccagccgtttgcccccctgttgtgttcatgggcacacaagtccccaaaggcgctgtcatgtttgtggtaccccagcccgttgtgcagagttcaaagcctccggtggtgagcccgaatggcaccagactctctcccattgcccctgctcctgggttttccccttcagcagcaaaagtcactcctcagattgattcatcaaggataaggagtcacatctgtagccacccaggatgtggcaagacatactttaaaagttcccatctgaaggcccacacgaggacgcacacaggagaaaagcctttcagctgtagctggaaaggttgtgaaaggaggtttgcccgttctgatgaactgtccagacacaggcgaacccacacgggtgagaagaaatttgcgtgccccatgtgtgaccggcggttcatgaggagtgaccatttgaccaagcatgcccggcgccatctatcagccaagaagctaccaaactggcagatggaagtgagcaagctaaatgacattgctctacctccaacccctgctcccacacagtgacagaccggaaagtgaagagtcagaactaactttggtctcagcgggagccagtggtgatgtaaaaatgcttccactgcaagtctgtggccccacaacgtgggcttaaagcagaagccccacagcctggcacgaaggccccgcctgggttaggtgactaaaagggcttcggccacaggcaggtcacagaaaggcaggtttcatttcttatcacataagagagatgagaaagcttttattcctttgaatattttttgaaggtttcagatgaggtcaacacaggtagcacagattttgaatctgtgtgcatatttgttactttacttttgctgtttatacttgagaccaacttttcaatgtgattcttctaaagcactggtttcaagaatatggaggctggaaggaaataaacattacggtacagacatggagatgtaaaatgagtttgtattattacaaatattgtcatctttttctagagttatcttctttattattcctagtctttccagtcaacatcgtggatgtagtgattaaatatatctagaactatcatttttacactattgtgaatatttggaattgaacgactgtatattgctaagagggcccaaagaattggaatcctccttaatttaattgctttgaaacatagctacaatttgtttttgcatttttgttttgaaagtttaacaaatgactgtatctaggcatttcattatgctttgaactttagtttgcctgcagtttcttgtgtagatttgaaaattgtataccaatgtgttttctgtagactctaagatacactgcactttgtttagaaaaaaaactgaagatgaaatatatattgtaaagaagggatattaagaatcttagataacttcttgaaaaagatggcttatgtcatcagtaaagtacctttatgttatgaggatataatgtgtgctttattgaattagaaaattagtgaccattattcacaggtggacaaatgttgatgttgtcctgttaatttataggcgttttttggggatgtggaggtagttgggtagaaaaattattagaacattcacttttgttaacagtatttctcttttattctgttatatagtggatgatatacacagtggcaaaacaaaagtacattgcttaaaatatatagtgaaaaatgtcactatatcttcccatttaacattgtttttgtatattgggtgtagatttctgacatcaaaacttggacccttggaaaacaaaagttttaattaaaaaaaatccttgtgacttacaatttgcacaatatttcttttgttgtactttatatcttgtttacaataaagaattccctttggtaaaaaaaaaaaaaaaaaaaaacaaaaaaaaaaaaaaaa

SEQ ID NO: 46 - protein, 469 aa, Homo sapiens
MEERMEMISERPKESMYSWNKTAEKSDFEAVEALMSMSCSWKSDFKKYVENRPVTPVSDLSEEENLLPGTPDFHTIPAFCLTPPYSPSDFEPSQVSNLMAPAPSTVHFKSLSDTAKPHIAAPFKEEEKSPVSAPKLPKAQATSVIRHTADAQLCNHQTCPMKAASILNYQNNSFRRRTHLNVEAARKNIPCAAVSPNRSKCERNTVADVDEKASAALYDFSVPSSETVICRSQPAPVSPQQKSVLVSPPAVSAGGVPPMPVICQMVPLPANNPVVTTVVPSTPPSQPPAVCPPVVFMGTQVPKGAVMFVVPQPVVQSSKPPVVSPNGTRLSPIAPAPGFSPSAAKVTPQIDSSRIRSHICSHPGCGKTYFKSSHLKAHTRTHTGEKPFSCSWKGCERRFARSDELSRHRRTHTGEKKFACPMCDRRFMRSDHLTKHARRHLSAKKLPNWQMEVSKLNDIALPPTPAPTQ

SEQ ID NO: 47 - protein, 452 aa, synthetic construct
QVQLVQSGAEVKKPGSSVKVSCKASGGTFSYYWIEWVRQAPGQGLEWMGEILPGSGTTNPNEKFKGRVTITADESTSTAYMELSSLRSEDTAVYYCARADYYGSDYVKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 214 aa, synthetic construct
DIQMTQSPSSLSASVGDRVTITCKASQHVITHVTWYQQKPGKAPKLLIYGTSYSYSGVPSRFSGSGSGTDFTLTISSLQPEDFATYYCQQFYEYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 5 aa, synthetic construct
YYWIE

SEQ ID NO: 50 - protein, 17 aa, synthetic construct
EILPGSGTTNPNEKFKG

SEQ ID NO: 51 - protein, 13 aa, synthetic construct
ADYYGSDYVKFDY

SEQ ID NO: 52 - protein, 11 aa, synthetic construct
KASQHVITHVT

SEQ ID NO: 53 - protein, 7 aa, synthetic construct
GTSYSYS

SEQ ID NO: 54 - protein, 9 aa, synthetic construct
QQFYEYPLT